(19) United States
(12) Patent Application Publication
(10) Pub. No.: US 2013/0243750 A1
(43) Pub. Date: Sep. 19, 2013

(54) ANTI-IGE ANTIBODIES AND METHODS USING SAME

(71) Applicant: GENENTECH, INC., South San Francisco, CA (US)

(72) Inventors: Heleen Scheerens, Sunnyvale, CA (US); Jeffrey Mark Harris, San Francisco, CA (US); John Matthews, San Francisco, CA (US); Wendy Sue Putnam, Belmont, CA (US); X. Charlene Liao, Palo Alto, CA (US)

(73) Assignee: Genentech, Inc., South San Francisco, CA (US)

(21) Appl. No.: 13/754,572

(22) Filed: Jan. 30, 2013

Related U.S. Application Data
(60) Provisional application No. 61/593,282, filed on Jan. 31, 2012.
(60) Provisional application No. 61/613,434, filed on Mar. 20, 2012.
(60) Provisional application No. 61/621,453, filed on Apr. 6, 2012.
(60) Provisional application No. 61/635,253, filed on Apr. 18, 2012.

(51) Int. Cl. C07K 16/42; A61K 45/06; A61K 39/395

(52) U.S. Cl. CPC C07K 16/4291; A61K 39/39566; A61K 45/06
USPC 424/131.1

(57) ABSTRACT

This invention provides anti-IgE antibodies that bind to the M1′ segment of a human IgE and their use in treating and preventing IgE-mediated disorders, as well as kits comprising the anti-IgE antibodies.

### CROSS-REFERENCE TO RELATED PATENT APPLICATIONS

This application claims the priority benefit of U.S. provisional application Ser. No. 61/593,282, filed Jan. 31, 2012, U.S. provisional application Ser. No. 61/613,434, filed Mar. 20, 2012, U.S. provisional application Ser. No. 61/621,453, filed Apr. 6, 2012, and U.S. provisional application Ser. No. 61/635,253, filed Apr. 18, 2012, all of which are incorporated herein by reference in their entirety.

### SEQUENCE LISTING

The present application contains a Sequence Listing which has been submitted in ASCII format via EFS-Web and is hereby incorporated by reference in its entirely. Said ASCII copy, created Jan. 30, 2013 is named P4867R1SeqList.txt and is 45,056 bytes in size.

### FIELD OF THE INVENTION

This invention relates to anti-IgE antibodies that bind to the M1′ segment of a human IgE and their use in treating and preventing IgE-mediated disorders.

### BACKGROUND OF THE INVENTION

Allergy refers to certain diseases in which immune responses to environmental antigens cause tissue inflammation and organ dysfunction. The clinical features of each allergic disease reflect the immunologically induced inflammatory response in the organ or tissue involved. These features are generally independent of the chemical or physical properties of the antigen. The diversity of allergic responses arises from the involvement of different immunological effector pathways, each of which generates a unique pattern of inflammation.

Allergy is common throughout the world. The predilection for specific diseases, however, varies among different age groups, sexes and races. The prevalence of sensitivity to specific allergens is determined both by genetic predilection and by the geographic and cultural factors that are responsible for exposure to the allergen. A clinical state of allergy affects only some individuals who encounter each allergen. The occurrence of allergic disease on exposure to an allergen requires not only prior “sensitization” but also other factors that determine the localization of the reaction to a particular organ.

A biological process that precedes the disease of allergy upon allergen exposure is an immune response known as “sensitization” or the sensitization phase. Once sensitization occurs, an individual does not become symptomatic until there is a subsequent exposure to the allergen. The effect of sensitization is also known as immune memory.

Elevated IgE levels are associated with allergic diseases including allergic asthma. IgE plays a central role in allergies by virtue of their role as allergen receptors on the surface of mast cells and basophils. IgE antibodies are fixed to the surface of mast cells and basophils at the Fc portion of the molecule to a high affinity cell surface receptor, called FcεRI. The allergic reaction is initiated when the polyvalent allergen molecule binds to antibodies that are occupying these receptors. The result is a bridging of the FcεRI, which in turn signals intracellularly causing the release and activation of mediators of inflammation: histamine, leukotrienes, chemotactic factors, platelet-activating factor, and proteinases. These activated mediators act locally and cause increased vascular permeability, vasodilation, smooth muscle contraction and mucous gland secretion. Such events are termed clinically the immediate or early phase, and occur within the first 15-30 minutes following allergen exposure. Over the succeeding 12 hours there is progressive tissue infiltration of inflammatory cells, proceeding from neutrophils to eosinophils to mononuclear cells in response to other chemical mediators not quite fully understood. This period of time 6-12 hours after allergen exposure is designated the late phase and is characterized by clinical manifestations of cellular inflammation. Given that late phase reactions, especially in the lung, occur in the absence of early phase reactions, it is still not entirely understood if the late phase reaction is necessarily IgE mediated. This mechanism is primarily responsible for the anaphylaxis, urticarial and the atopic diseases such as allergic rhinitis, allergic asthma, atopic dermatitis and allergic gastroenteropathy.

IgE exists in a membrane bound form and in a secreted form. These distinct forms appear to be splice variants. Previous approaches to achieve therapeutic effect by down regulating IgE targeted primarily the secreted form (e.g., XOLAIR® omalizumab), so as to prevent or disarm further “arming” of the immune system. The secreted form of IgE is a shorter form, essentially the Fc region ends at the CH4 domain, whereas the longer form includes additional C-terminal residues including the peptides encoded by the exons known as M1/M1′ and M2. Conventional therapy with anti-IgE antibodies, which bind to the secreted form of IgE, results in reduction of secreted serum IgE (total IgE not complexed to Xolair). Casale et al., J. Allergy Clin. Immunol. 100 (1): 110-121 (1997).

Membrane bound IgE which includes the M1′ section is present in human IgE-switched B cells, IgE memory B cell, and IgE plasmablasts. U.S. Pat. No. 8,071,097 (also described in WO2008/116149, the disclosures both of which are incorporated by reference in their entirety) discloses antibodies that target the M1′ segment of membrane bound IgE. These antibodies may deplete M1′ expressing B cells via apoptosis and/or antibody-dependent cell-mediated cytotoxicity mechanism.

All references cited herein, including patent applications and publications, are hereby incorporated by reference in their entirety.

### SUMMARY OF THE INVENTION

Provided herein is a method of treating or preventing an IgE-mediated disorder comprising administering to a human patient an effective amount of an anti-IgE antibody that binds the M1′ segment of a human IgE, wherein an interval between administrations of the antibody is about one month or longer. In some embodiments, the interval between administrations is about two months, about three months, about four months, about five months, about six months, or longer. In some embodiments, the interval between administrations is about three months with an additional administration at week 4 after the first administration. In some embodiments, the antibody is administered at a dosage from about 150 mg to about 450 mg per dose. In some embodiments, the antibody is administered at a dosage of about 150 mg per dose, about 300 mg per dose, or about 450 mg per dose. In some embodiments, the antibody is administered subcutaneously or intravenously. In some embodiments, the serum total IgE in the human patient is reduced relative to baseline after the antibody treatment. In some embodiments, the allergen-specific IgE in the human patient is reduced relative to baseline after the antibody treatment. In some embodiments, an allergen-induced increase in serum total IgE in the human patient is prevented or reduced after the antibody treatment.

In some embodiments, an allergen-induced increase in allergen-specific IgE in the human patient is prevented or reduced after the antibody treatment. In some embodiment, the production of new IgE is prevented or reduced after antibody treatment.

Also provided herein is a method of treating or preventing an IgE-mediated disorder comprising administering to a human patient an effective amount of an anti-IgE antibody that binds the M1′ segment of a human IgE, wherein the antibody is administered at a dose of about 150 mg to about 450 mg per dose. In some embodiments, the antibody is administered at a dosage about 150 mg/per dose, about 300 mg/per dose, or about 450 mg per dose. In some embodiments, the antibody is administered subcutaneously or intravenously.

Also provided herein is a method of reducing serum total IgE and/or allergen-specific IgE in a human relative to baseline comprising administering to a human patient an effective amount of an anti-IgE antibody that binds the M1′ segment of a human IgE, wherein an interval between administrations of the antibody is about one month or longer. In some embodiments, the serum total IgE is reduced by at least about 20% from the baseline level. In some embodiments, the serum total IgE is reduced by at least about 25% from the baseline level. In some embodiments, the reduction of the serum total IgE is sustained for at least one month, at least two months, at least three months, at least four months, at least five months, or at least six months after the last administration of the antibody. Also provided herein is a method of preventing the production of new IgE comprising administering to a human patient an effective amount of an anti-IgE antibody that binds the M1′ segment of a human IgE.

Also provided herein is a method of preventing or reducing an allergen-induced increase in serum total IgE and/or in allergen-specific IgE in a human patient comprising administering to a human patient an effective amount of an anti-IgE antibody that binds to the M1′ segment of a human IgE. In some embodiments, an interval between administrations of the antibody is about one month or longer. In some embodiments, an interval between administrations of the antibody is about two months. In some embodiments, an interval between administrations of the antibody is about three months. In some embodiments, an interval between administrations of the antibody is about four months. In some embodiments, an interval between administrations of the antibody is about five months. In some embodiments, an interval between administrations of the antibody is about six months. In some embodiments, the antibody is administered at a dose of about 150 to about 450 mg per dose. In some embodiments, the allergen-induced increase in allergen-specific IgE is prevented or reduced. In some embodiments, the prevention or reduction of allergen-allergen induced increase in serum total IgE and/or allergen-specific IgE is sustained for at least one month, at least two months, at least three months, or at least six months after the last administration of antibody.

In some embodiments of the methods described herein, the antibody is administered for treating an IgE-mediated disorder selected from the group consisting of: allergic rhinitis, allergic asthma, non-allergic asthma, atopic dermatitis, allergic gastroenteropathy, anaphylaxis, urticaria, food allergies, allergic bronchopulmonary aspergillosis, parasitic diseases, interstitial cystitis, hyper-IgE syndrome, ataxia-telangiectasia, Wiskott-Aldrich syndrome, athymic lymphoplasia, IgE myeloma, graft-versus-host reaction and allergic purpura. In some embodiments, the IgE-mediated disorder is allergic rhinitis, allergic asthma, or non-allergic asthma. In some embodiments, the method described herein is for treating a human patient having allergic asthma that is inadequately controlled by standard of care, e.g., a high-dose inhaled or oral corticosteroids in combination with a second controller. In some embodiments, the method described herein is for treating a human patient having severe, moderate, or mild asthma. In some embodiments, the method described herein is for treating a human patient with allergic asthma inadequately controlled despite high dose inhaled corticosteroids (ICS) (≧400 μg/day total daily dose of fluticasone propionate (FP) or equivalent) and a second controller (e.g., after as least 12 weeks or at least 36 weeks of treatment). In some embodiments, the second controller is a bronchodilator or an anti-leukotriene agent.

In some embodiments, the method described herein further comprises administering to the human patient a second drug in conjunction with the antibody for treating or preventing an IgE-mediated disorder, wherein the second drug is selected from the group consisting of: an anti-IgE antibody, an antihistamine, a bronchodilator, a glucocorticoid, an NSAID, a decongestant, a cough suppressant, an analgesic, a TNF-antagonist, an integrin antagonist, an immunosuppressive agent, an IL-4 antagonist, an IL-13 antagonist, a dual IL-4/IL-13 antagonist, a DMARD, an antibody that binds to a B-cell surface marker, and a BAFF antagonist. In some embodiments of the methods described herein, the antibody is administered to the human patient in conjunction with a second method treatment for an IgE-mediated disorder. In some embodiments, the second method of treatment comprises a treatment regimen of allergen desensitization.

In the methods described herein, any of the anti-IgE antibodies described herein may be administered to the human patient. In some embodiments, the anti-IgE antibody is a chimeric, a humanized, or a human antibody. In some embodiments, the antibody specifically binds an epitope in the M1′ segment of a human IgE shown in FIG. 14. In some embodiments, the anti-IgE antibody specifically binds to the same epitope as one bound by an antibody selected from the group consisting of: 47H4, 7A6, 26A11, 47H4v5, 7A6v1 and 26A11v6. In some embodiments, the epitope corresponds to a peptide having the amino acid sequence selected from the group consisting of: SEQ ID NO:4, SEQ ID NO:5, SEQ ID NO:6, and SEQ ID NO:7. In some embodiments, the antibody specifically binds an epitope in the M1′ segment of IgE defined by the residues 317 to 352 of SEQ ID NO:1. In some embodiments, the antibody specifically binds an epitope in the M1′ segment of IgE defined by the residues 317 to 352 of SEQ ID NO:1 and has a Scatchard binding affinity that is equivalent to that of the murine anti-IgE antibody 47H4. In some embodiments, the affinity is between 0.30 and 0.83 nM. In some embodiments, the antibody specifically binds an epitope in the M1′ segment of IgE defined by the residues 317 to 352 of SEQ ID NO:1 and has a Scatchard binding affinity that is equivalent to that of anti-IgE antibody 47H4v5. In some embodiments, the affinity is about 1.5 nM. In some embodiments, the antibody comprises the heavy chain and light chain HVRs of an antibody or antigen-binding fragment thereof selected from the group consisting of: 26A11, 26A11 v.1-16, 7A6, 7A6v1, 47H4, and 47H4v1-6. In some embodiments, the antibody comprises heavy and light variable regions of the heavy and light chains of the antibody or antigen-binding fragment thereof selected from the group consisting of: 26A11, 26A11 v.1-16, 7A6, 7A6v1, 47H4, 47H4v1-6.

In some embodiments, the anti-IgE antibody comprises a heavy chain and a light chain variable region, wherein the heavy chain variable region comprises the amino acid sequence of SEQ ID NO:29 and the light chain variable region comprises the amino acid sequence of SEQ ID NO:19. In some embodiments, the antibody comprises a heavy chain and a light chain variable region, wherein the heavy chain variable region comprises an HVR-H1, HVR-H2 and HVR-H3, and the light chain variable region comprises HVR-L1, HVR-L2 and HVR-L3, and wherein (a) the HVR-H1 comprises residues 26-35 of SEQ ID NO:29, (b) the HVR-H2 comprises residues 49-66 of SEQ ID NO:29, (c) the HVR-H3 comprises residues 97-106 of SEQ ID NO:29, (d) the HVR-L1 comprises residues 24-39 of SEQ ID NO:19, (e) the HVR-L2 comprises residues 55-61 of SEQ ID NO:19, and (f) the HVR-L3 comprises residues 94-102 of SEQ ID NO:19. The In some embodiments, the antibody further comprises a human consensus framework. In some embodiments, the heavy chain variable region of the antibody comprises a subgroup III consensus framework. In some embodiments, the light chain variable region comprises a kappa subgroup I consensus framework. In some embodiments, the anti-IgE antibody administered to a human patient comprises a heavy chain and a light chain variable region, wherein the heavy chain variable region comprises the amino acid sequence of SEQ ID NO:39 and the light chain variable region comprises the amino acid sequence of SEQ ID NO:40. In some embodiments, an antigen-binding fragment of an anti-IgE antibody is administered to a human patient, wherein the anti-IgE antibody comprises a heavy chain and a light chain variable region, wherein the heavy chain variable region comprises the amino acid sequence of SEQ ID NO:39 and the light chain variable region comprises the amino acid sequence of SEQ ID NO:40.

In some embodiments, the anti-IgE antibody has ADCC activity. In some embodiments, the anti-IgE antibody is afucosylated. In some embodiments, the anti-IgE antibody depletes IgE-switched B-cells. In some embodiments, the anti-IgE antibody depletes IgE memory B-cells. In some embodiments, the anti-IgE antibody depletes IgE plasmablast. In some embodiments, the anti-IgE antibody is in pharmaceutical composition comprising the antibody and a pharmaceutically acceptable carrier.

Also provided herein is a kit comprising an anti-IgE antibody that binds the M1′ segment of a human IgE and a package insert indicating that the antibody is administered to a human patient for treating an IgE-mediated disorder, wherein an interval between administrations of the antibody to the human patient is about one month or longer.

Also provided here is a kit comprising an anti-IgE antibody that binds the M1′ segment of a human IgE and a package insert indicating that the antibody is administered to a human patient for treating an IgE-mediated disorder, wherein the antibody is administered at a dosage from about 150 mg to about 450 mg per dose.

In some embodiments, the package insert in the kit further indicates that the treatment is effective in reducing serum total IgE relative to baseline in a human patient. In some embodiments, the package insert in the kit further indicates that the treatment is effective in reducing allergen-specific IgE relative to baseline in a human patient. In some embodiments, the package insert further indicates that the treatment is effective in preventing or reducing an allergen-induced increase in serum total IgE in a human patient. In some embodiments, the package insert further indicates that the treatment is effective in preventing or reducing an allergen-induced increase in allergen-specific IgE in a human patient. In some embodiments, the human patient has severe, moderate, or mild asthma. In some embodiments, the antibody in the kit is in a vial. In some embodiments, the antibody in the kit is in a pre-filled syringe. In some embodiment, the kit further comprises an injection device (such as an auto-injector).

In some embodiments, the kit further comprises a second drug selected from the group consisting of: an anti-IgE antibody, an antihistamine, a bronchodilator, a glucocorticoid, an NSAID, a decongestant, a cough suppressant, an analgesic, a TNF-antagonist, an integrin antagonist, an immunosuppressive agent, an IL-4 antagonist, an IL-13 antagonist, a dual IL-4/IL-13 antagonist, a DMARD, an antibody that binds to a B-cell surface marker, and a BAFF antagonist, and a package insert indicating administration of the antibody to a human patient at monthly or quarterly intervals in conjunction with the second drug for treating an IgE-mediated disorder. In some embodiments, the second drug is anti-IgE antibody rhuMAbE25.

In some embodiment, the invention provides for any of the prior described anti-IgE-M1′ antibodies for use in any of the prior described methods, wherein the antibodies are administered by any of the prior described dosing interval, amounts or regimens.

In some embodiment, the invention provides for any of the prior described anti-IgE-M1′ antibodies for use in any of the prior described methods, wherein the antibodies are prepared to be administered by any of the prior described dosing interval, amounts or regimens.

In some embodiment, the invention provides for the use of any of the prior described anti-IgE-M1′ antibodies any of the prior described methods, wherein the antibodies are administered is any of the prior described dosing intervals, amounts or regimens.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 is a diagram of the Phase 1a single ascending-dose study in healthy volunteers.

FIG. 2 is a diagram of the Phase 1b multiple ascending dose study in patients with mild asthma.

FIG. 3 is a graph demonstrating mean serum concentration over time in all cohorts in the Phase 1a study.

FIG. 4 is a graph demonstrating mean serum concentration over time in all cohorts in the Phase 1b study.

FIG. 5 is graph demonstrating serum total IgE at 85 and 168 days after treatment with MEMP1972A. Data are presented for study Days 85 (A) and 168 (B), with study Day 1 defined as the day of the single-dose administration. Data are expressed as % change from the baseline, where baseline is defined as the average of pre-dose visits; mean±SD, n=3-4 patients in 0.003, 0.03 and 0.3 mg/kg IV groups, n=5 in 1, 3, 5 mg/kg IV and 3 mg/kg SC group, and n=14 in placebo group.

FIG. 6 is a graph demonstrating serum total IgE in patients with allergic rhinitis after treatment with MEMP1972A. Data are expressed as % change from baseline, where baseline is defined as the average of pre-dose visits; mean±SD, n=8 patients in MEMP1972A groups and n=12 in placebo group (IV and SC combined).

FIG. 7 is a diagram of the Phase 2a proof-of-activity allergen challenge study in patients with mild asthma.

FIG. 8 is a graph demonstrating that the anti-M1 prime antibody (MEMP1972A) reduced both early asthmatic reactions (EAR) and late asthmatic reactions (LAR) as measured by percent decline in forced expiratory volume in 1 second (FEV₁) over time following allergen inhalation in a Phase 2a study. A) shows the mean percent FEV₁ as compared to baseline (pre-challenge) over time following the allergen inhalation in patients at screening before placebo or drug administration. B) shows mean percent FEV₁ as compared to baseline (pre-challenge) over time following the allergen inhalation at Day 86 in patients with placebo or the drug (MEMP1972A).

FIG. 9 is a graph demonstrating that the anti-M1 prime antibody prevented allergen-induced increase in allergen-specific IgE and reduced total IgE in patients with mild asthma in a Phase 2a study. A) shows allergen-specific IgE (to challenge allergens) in patients treated with placebo or Anti-M1 prime (MEMP1972A). *p≦0.01; †p≦0.05. B) shows allergen-specific IgE (to irrelevant allergens (i.e., non-challenge allergens)) in patients subjected to whole lung allergen challenge and treated with placebo or Anti-M1 prime (MEMP1972A). C) shows total IgE in patients treated with placebo or Anti-M1 prime in interim results. D) shows total IgE in patients treated with placebo or Anti-M1 prime. IgE levels before start of study set as baseline of 100% (MEMP1972A). *p≦0.01. Data shown as mean±SEM. For A)-C), not all subjects included in late time points; n=4-5/treatment group on Day 197.

FIG. 10 is a graph showing that MEMP1972A reduced allergen challenge-induced increases in eosinophils. A) shows sputum eosinophil levels at screening. B) shows sputum eosinophil levels at week 12 of the study. Data are presented as mean±standard error.

FIG. 11 is a graph showing reduction of peripheral blood eosinophils in patients treated with MEMP1972A. A) shows percentage of baseline (screening) in eosinophils (%). *p≦0.10; †p≦0.15. B) shows percentage of baseline (screening) in eosinophils (absolute count). *p≦0.10; †p≦0.15. Data are presented as mean±standard error.

FIG. 12 is a graph showing MEMP1972A blocks increases in allergen-induced CCL17 levels. A) shows CCL17 levels as a percentage of baseline over the course of the study. B) shows CCL17 levels as a percentage of baseline at screening and Day 86. Data are presented as mean±standard error.

FIG. 13 is a diagram of the Phase 2b study in patients with asthma. Patients in the placebo group receive a total of nine placebo doses at monthly intervals (weeks 0, 4, 8, 12, 16, 20, 24, 28, and 32). Patients in the 300 mg anti-M1 prime antibody arm receive a total of nine doses of the antibody at monthly intervals (weeks 0, 4, 8, 12, 16, 20, 24, 28, and 32). Patients in the 150 mg and 450 mg anti-M1 prime antibody arms receive a total of four active doses of the antibody including three doses at quarterly intervals (weeks 0, 12, and 24) as well as an extra dose at week 4 and the remaining five doses are placebo.

FIGS. 14A-B is an alignment of selected constant chain regions of IgE of the human (SEQ ID NO:1), rhesus monkey (SEQ ID NO:2) and cynomolgous monkey (SEQ ID NO:3). Shown are the approximate locations of the CH2, CH3, CH4, M1′, transmembrane and intracellular domains. FIGS. 15A-F show the variable light and heavy chain sequences of murine antibody 26A11, 7A6 and 47H4 and various humanized variants thereof. Positions are numbered according to Kabat and hypervariable regions that were grafted to the variable consenus network (Kappa I for light, subgroup III for heavy chain) are boxed. A) shows, relative to human kappa I light chain (SEQ ID NO:8), the variable light chain of 26A11 (SEQ ID NO:9) and humanized variants 1,4 (SEQ ID NO:10), variants 2,5 (SEQ ID NO:11), variants 3,6 (SEQ ID NO:12), variants 13,15 (SEQ ID NO:13) and variants 14,16 (SEQ ID NO:14). B) shows, relative to human kappa I light chain (SEQ ID NO:8), the variable light chain of 7A6 (SEQ ID NO:15) and humanized variant 1 (SEQ ID NO:16). C) shows, relative to human kappa I light chain (SEQ ID NO:8), the variable light chain of 47H4 (SEQ ID NO:17) and humanized variants 1,3 (SEQ ID NO:18) and variants 2, 4-6 (SEQ ID NO:19). D) shows, relative to human III heavy chain (SEQ ID NO:20), the variable heavy chain of 26A11 (SEQ ID NO:21) and humanized variants 1-3, 13, 14 (SEQ ID NO:22) and variants 4-6, 15, 16 (SEQ ID NO:23). E) shows, relative to human heavy chain (SEQ ID NO:20), the variable heavy chain of 7A6 (SEQ ID NO:24) and humanized variant 1 (SEQ ID NO:25). F) shows, relative to human III heavy chain (SEQ ID NO:20), the variable heavy chain of 47H4 (SEQ ID NO:26), and humanized variants 1,2 (SEQ ID NO:27), variants 3-4 (SEQ ID NO:28), variant 5 (SEQ ID NO:29) and variant 6 (SEQ ID NO:30).

FIG. 16 is a graph demonstrating that the anti-M1 prime antibody reduced challenge and non-challenge specific IgE in patients with mild asthma in a Phase 2a study. A) shows allergen-specific IgE to challenge allergens in patients treated with placebo or Anti-M1 prime (MEMP1972A). B) shows allergen-specific IgE to non-challenge allergens in patients treated with placebo or Anti-M1 prime (MEMP1972A). IgE levels are shown as percent of baseline, and IgE levels before start of study is the baseline (100%). Data shown as mean or median.

### DETAILED DESCRIPTION OF THE INVENTION

The present application provides methods of treating or preventing an IgE-mediated disorder using an anti-IgE antibody that binds to the M1′ segment of an IgE. The inventors have shown in clinical studies that a humanized anti-M1′ antibody was effective in reducing serum total IgE and allergen specific IgE in healthy subjects, and patients with allergic rhinitis or allergic asthma, and such reduction of total IgE was sustained for at least three months after the last dose in both the single and multiple dose studies. In addition, in a separate study, the inventors have shown that an allergen-induced increase in serum total IgE and in allergen-specific IgE was prevented or reduced in patients after the anti-IgE antibody treatment.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

An “allergen” or “immunogen’ is any molecule that can trigger an immune response. As used herein, the term covers either the antigenic molecule itself, or its source, such as pollen grain, animal dander, insect venom or food product. This is contrasted with the term antigen, which refers to a molecule can be specifically recognized by an immunoglobulin or T-cell receptor. Any foreign substance capable of inducing an immune response is a potential allergen. Many different chemicals of both natural and synthetic origin are known to be allergenic. Complex natural organic chemicals, especially proteins, are likely to cause antibody-mediated allergy, whereas simple organic compounds, inorganic chemicals, and metals more preferentially cause T-cell mediated allergy. In some cases, the same allergen may be responsible for more than one type of allergy. Exposure to the allergen may be through inhalation, injection, injection, or skin contact.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes.

Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2. An “isolated” antibody is one that has been identified, separated and/or recovered from a component of its production environment (E.g., naturally or recombinantly). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Competition assays are known in the art.

In an exemplary competition assay, immobilized the M1′ segment of IgE is incubated in a solution comprising a first labeled antibody that binds to the M1′ segment (e.g., antibody 47H4, 47H4 v1, v2, v3, v4, v5 or v6) and a second unlabeled antibody that is being tested for its ability to compete with the first antibody for binding to the M1′ segment. The second antibody may be present in a hybridoma supernatant. As a control, immobilized M1′ segment is incubated in a solution comprising the first labeled antibody but not the second unlabeled antibody. After incubation under conditions permissive for binding of the first antibody to M1′ segment, excess unbound antibody is removed, and the amount of label associated with immobilized M1′ segment is measured. If the amount of label associated with immobilized M1′ segment is substantially reduced in the test sample relative to the control sample, then that indicates that the second antibody is competing with the first antibody for binding to M1′ segment. See Harlow and Lane (1988) Antibodies: A Laboratory Manual ch. 14 (Cold Spring Harbor Laboratory, Cold Spring Harbor, N.Y.).

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L34 | L30-L36 | |
| L2 | L50-L56 | L50-L56 | L50-L56 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H56 | H47-H58 | |
| H3 | H95-H102 | H95-H102 | H95-H102 | H93-H101 | |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 47-65 (a preferred embodiment) (H2), and 93-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “acceptor human framework” for the purposes herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). In one embodiment, for the VL, the subgroup is subgroup kappa I as in Kabat et al., supra. In one embodiment, for the VH, the subgroup is subgroup III as in Kabat et al., supra.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences:

(SEQ ID NO: 31)
EVQLVESGGGLVQPGGSLRLSCAAS (H1),
(SEQ ID NO: 32)
WVRQAPGKGLEWVA (H2),
(SEQ ID NO: 33)
RFTISRDDSKNTLYLQMNSLRAEDTAVYYCAR (H3),
(SEQ ID NO: 34)
WGQGTLVTVSS (H4).

A “VL subgroup I consensus framework” comprises the consensus sequence obtained from the amino acid sequences in variable light kappa subgroup I of Kabat et al., supra. In one embodiment, the VH subgroup I consensus framework amino acid sequence comprises at least a portion or all of each of the following sequences:

(SEQ ID NO: 35)
DIQMTQSPSSLSASVGDRVTITC (L1),
(SEQ ID NO: 36)
WYQQKPGKAPKLLIY (L2),
(SEQ ID NO: 37)
GVPSRFSGSGSGTDFTLTISSLQPEDFATYYC (L3),
(SEQ ID NO: 38)
FGQGTKVEIKR (L4).

An “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al., Proc Nat. Acad. Sci. USA 91:3809-3813 (1994); Schier et al., Gene 169:147-155 (1995); Yelton et al., J. Immunol. 155:1994-2004 (1995); Jackson et al., J. Immunol. 154(7):3310-9 (1995); and Hawkins et al., J. Mol. Biol. 226:889-896 (1992).

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the M1′ specific antibodies of the present invention are specific to the M1′ extracellular segment of IgE found on membrane IgE on B-cells, but which is not present on secreted IgE. In some embodiments, the antibody that binds to the M1′ segment of IgE has a dissociation constant (Kd) of ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

Antibodies that “induce apoptosis” or are “apoptotic” are those that induce programmed cell death as determined by standard apoptosis assays, such as binding of annexin V, fragmentation of DNA, cell shrinkage, dilation of endoplamic reticulum, cell fragmentation, and/or formation of membrane vesicles (called apoptotic bodies). For example, the apoptotic activity of the anti-IgE antibodies of the present invention can be showed by staining cells with surface bound IgE with annexin V.

The term “total serum IgE” or “serum total IgE” refers to a total amount of IgE present in a serum sample.

The term “allergen-specific IgE” refers to IgE that is specific to a particular antigen, resulting from an initial exposure to allergen in a process known as allergy sensitization, and which binds the surface of mast cells and basophils and which can result in the activation of mast cells and basophils upon subsequent exposure to the same allergen. Several mitogenic factors in viruses (e.g., Cytomegalovirus—CMV), bacteria (e.g., Staphylococcus), helminths (e.g., Ascaris, Schistosoma) and adjuvant factors in air pollution (e.g., cigarette smoke, and diesel exhaust) stimulate the production of IgE molecules without initiating any allergen specific IgE-sensitization. Thus, because IgE levels may elevate in a manner that does not necessarily predispose the host to become more susceptible to an IgE-mediated disorder, allergen-specific IgE levels are sometimes used in clinical evaluations.

As used herein, a “baseline” level (such as baseline level for serum total IgE, and allergen-specific IgE) in a human refers to the level before an administration of an anti-IgE antibody described herein to the human.

The term “deplete IgE-M1 prime expressing B-cells” means the ability to deplete one or more of IgE-switched B-cells, IgE plasmablasts, or IgE memory B cells, consequently reducing the population of or effectiveness of B-cells that specifically secrete IgE (i.e., plasma cells), but does not significantly affect the population or effectiveness of B-cells that secrete other immunoglobulins, such as IgG1, IgG2, IgG3, IgG4, IgA and IgM.

The term “solid phase” describes a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2, IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (see M. Daëron, Annu. Rev. Immunol. 15:203-234 (1997)). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils, with PBMCs and MNK cells being preferred. The effector cells may be isolated from a native source, e.g., blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed.

Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

The carbohydrate attached to the Fc region may be altered. Native antibodies produced by mammalian cells typically comprise a branched, biantennary oligosaccharide that is generally attached by an N-linkage to Asn297 of the CH2 domain of the Fc region. See, e.g., Wright et al. (1997) TIBTECH 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an IgG may be made in order to create IgGs with certain additionally improved properties.

For example, antibody modifications are provided having a carbohydrate structure that lacks fucose attached (directly or indirectly) to an Fc region. Such modifications may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody modifications include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. MoL Biol. 336: 1239-1249 (2004); Yamane-Ohnuki et al. Biotech. Bioeng. 87: 614 (2004). Examples of cell lines capable of producing defucosylated antibodies include Lee 13 CHO cells deficient in protein fucosylation (Ripka et al. Arch. Biochem. Biophys. 249:533-545 (1986); US Pat. Appl. Pub. No. 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al, Biotechnol. Bioeng., 94(4):680-688 (2006); and WO2003/085107).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays. According to another embodiment, the Kd is measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, authored by Genentech, Inc. The source code of ALIGN-2 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Unless specifically stated otherwise, all % amino acid sequence identity values used herein are obtained as described in the immediately preceding paragraph using the ALIGN-2 computer program.

An “isolated” nucleic acid molecule encoding the antibodies herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. parenteral administration) to a patient to be treated with the protein of interest and, in certain embodiments of the invention, may be one which is suitable for subcutaneous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present invention are hypertonic as a result of the addition of salt and/or buffer.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

A “pharmaceutically acceptable acid” includes inorganic and organic acids which are non toxic at the concentration and manner in which they are formulated. For example, suitable inorganic acids include hydrochloric, perchloric, hydrobromic, hydroiodic, nitric, sulfuric, sulfonic, sulfinic, sulfanilic, phosphoric, carbonic, etc. Suitable organic acids include straight and branched-chain alkyl, aromatic, cyclic, cycloaliphatic, arylaliphatic, heterocyclic, saturated, unsaturated, mono, di- and tri-carboxylic, including for example, formic, acetic, 2-hydroxyacetic, trifluoroacetic, phenylacetic, trimethylacetic, t-butyl acetic, anthranilic, propanoic, 2-hydroxypropanoic, 2-oxopropanoic, propandioic, cyclopentanepropionic, cyclopentane propionic, 3-phenylpropionic, butanoic, butandioic, benzoic, 3-(4-hydroxybenzoyl)benzoic, 2-acetoxy-benzoic, ascorbic, cinnamic, lauryl sulfuric, stearic, muconic, mandelic, succinic, embonic, fumaric, malic, maleic, hydroxymaleic, malonic, lactic, citric, tartaric, glycolic, glyconic, gluconic, pyruvic, glyoxalic, oxalic, mesylic, succinic, salicylic, phthalic, palmoic, palmeic, thiocyanic, methanesulphonic, ethanesulphonic, 1,2-ethanedisulfonic, 2-hydroxyethanesulfonic, benzenesulphonic, 4-chorobenzenesulfonic, napthalene-2-sulphonic, p-toluenesulphonic, camphorsulphonic, 4-methylbicyclo[2.2.2]-oct-2-ene-1-carboxylic, glucoheptonic, 4,4′-methylenebis-3-(hydroxy-2-ene-1-carboxylic acid), hydroxynapthoic.

“Pharmaceutically-acceptable bases” include inorganic and organic bases which are non-toxic at the concentration and manner in which they are formulated. For example, suitable bases include those formed from inorganic base forming metals such as lithium, sodium, potassium, magnesium, calcium, ammonium, iron, zinc, copper, manganese, aluminum, N-methylglucamine, morpholine, piperidine and organic nontoxic bases including, primary, secondary and tertiary amines, substituted amines, cyclic amines and basic ion exchange resins, [e.g., N(R′)₄⁺ (where R′ is independently H or C₁₋₄ alkyl, e.g., ammonium, Tris)], for example, isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional pharmaceutically acceptable acids and bases useable with the present invention include those which are derived from the amino acids, for example, histidine, glycine, phenylalanine, aspartic acid, glutamic acid, lysine and asparagine.

“Pharmaceutically acceptable” buffers and salts include those derived from both acid and base addition salts of the above indicated acids and bases. Specific buffers and/or salts include histidine, succinate and acetate.

A “pharmaceutically acceptable sugar” is a molecule which, when combined with a protein of interest, significantly prevents or reduces chemical and/or physical instability of the protein upon storage. When the formulation is intended to be lyophilized and then reconstituted, “pharmaceutically acceptable sugars” may also be known as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol. As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. In some embodiments, the treatment improves asthma control, reduces asthma exacerbations, improves lung function, and/or improves patient reported symptoms. An individual is successfully “treated”, for example, if one or more symptoms associated with an IgE-mediated disorder are mitigated or eliminated.

As used herein, “in conjunction with” refers to administration of one treatment modality in addition to another treatment modality. As such, “in conjunction with” refers to administration of one treatment modality before, during or after administration of the other treatment modality to the individual.

As used herein, the term “prevention” includes providing prophylaxis with respect to occurrence or recurrence of a disease in an individual. An individual may be predisposed to, susceptible to an IgE-mediated disorder, or at risk of developing an IgE-mediated disorder, but has not yet been diagnosed with the disorder. In some embodiments, anti-IgE antibodies described herein are used to delay development of an IgE-mediated disorder. In some embodiments, the anti-IgE antibodies described herein prevents asthma exacerbations and/or decline in lung function or asthma states. In some embodiments, the anti-IgE antibodies described herein prevent IgE-mediated immune response.

As used herein, an individual “at risk” of developing an IgE-mediated disorder may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of the IgE-medicated disorder, as known in the art. An individual having one or more of these risk factors has a higher probability of developing the disorder than an individual without one or more of these risk factors.

An “effective amount” refers to at least an amount effective, at dosages and for periods of time necessary, to achieve the desired or indicated effect, including a therapeutic or prophylactic result. An effective amount can be provided in one or more administrations.

A “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

“Chronic” administration refers to administration of the medicament(s) in a continuous as opposed to acute mode, so as to main the initial therapeutic effect (activity) for an extended period of time.

“Intermittent” administration is treatment that is not consecutively done without interruption, but rather is cyclic in nature.

As used herein, an “individual” or a “subject” is a mammal. A “mammal” for purposes of treatment refers to any animal classified as a mammal, including humans, domestic and farm animals, and zoo, sports, or pet animals, such as dogs, horses, rabbits, cattle, pigs, hamsters, gerbils, mice, ferrets, rats, cats, etc. Preferably, the mammal is human.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile.

A “sterile” formulation is aseptic or free from all living microorganisms and their spores.

An “antihistamine” as used herein is an agent that antagonizes the physiological effect of histamine. The binding of histamine to its receptors, H₁ and H₂ results in the characteristic allergic symptoms and effects or itching, redness, swelling etc. Many antihistamines act by blocking the binding of histamine to its receptors, H₁, H₂; however others are believed to operate by inhibiting the release of histamine. Examples of antihistamines are chlorpheniramine, diphenhydramine, promethazine, cromolyn sodium, astemizole, azatadine maleate, bropheniramine maleate, carbinoxamine maleate, cetirizine hydrochloride, clemastine fumarate, cyproheptadine hydrochloride, dexbrompheniramine maleate, dexchlorpheniramine maleate, dimenhydrinate, diphenhydramine hydrochloride, doxylamine succinate, fexofendadine hydrochloride, terphenadine hydrochloride, hydroxyzine hydrochloride, loratidine, meclizine hydrochloride, tripelannamine citrate, tripelennamine hydrochloride, triprolidine hydrochloride.

A “bronchodilator” as used herein, describes agents that antagonize or reverse bronchoconstriction, a physiological event that occurs typically in early phase asthmatic reactions resulting in decreased lung capacity and shortness of breath. Example bronchodilators include epinephrine, a broad acting alpha and beta-adrenergic, and the beta-adrenergics, albuterol, pirbuterol, metaproterenol, salmeterol, and isoetharine. Bronchodilation can also be achieved through administration of xanthines, including aminophylline and theophylline.

A “non-steroidal anti-inflammatory drug” or “NSAID”, as used herein describes agents having anti-inflammatory activity that are not steroidal based. Example NSAID's include acematacin, acetaminophen, aspirin, azapropazone, benorylate, bromfenac sodium, cyclooxygenase (COX)-2 inhibitors such as GR 253035, MK966, celecoxib (CELEBREX®; 4-(5-(4-methylphenyl)-3-(trifluoromethyl)-1H-pyrazol-1-yl)benzene-sulfonamide) and valdecoxib (BEXTRA®), diclofenac, diclofenac retard, diclofenac sodium, diflunisal, etodolac, fenbufen, fenoprofen calcium, flurbiprofen, ibuprofen, ibuprofen retard, indomethacin, ketoprofen, meclofenamate sodium, mefenamic acid, meloxicam (MOBIC®), nabumetone, naproxen, naproxen sodium, oxyphenbutazone, phenylbutzone, piroxicam, sulindac, tenoxicam, tiaprofenic acid, tolmetin, tolmetin sodium, including salts and derivatives thereof, etc.

The term “IgE-mediated disorders” are disorders associated with excess IgE levels or activity in which atypical symptoms may manifest due to levels of IgE locally and/or systemically in the body. Such disorders include, asthma, atopic dermatitis, allergic rhinitis, fibrosis (e.g., pulmonary fibrosis, such as IPF). IgE-mediated disorders include atopic disorders, which are characterized by a general inherited propensity to respond immunologically to many common naturally occurring inhaled and ingested antigens and the continual production of IgE antibodies. Specific atopic disorders include allergic asthma, allergic rhinitis (conjunctivitis), atopic dermatitis, food allergy, anaphylaxis, contact dermatitis, allergic gastroenteropathy, allergic bronchopulmonary aspergillosis and allergic purpura (Henoch-Schönlein). Atopic patients often have multiple allergies, meaning that they have IgE antibodies to, and symptoms from, many environmental allergens, including seasonal, perennial and occupational allergens. Example seasonal allergens include pollens (e.g., grass, tree, rye, timothy, ragweed), while example perennial allergens include fungi (e.g., molds, mold spores), feathers, animal (e.g., pet or other animal dander) and insect (e.g., dust mite) debris. Example occupational allergens also include animal (e.g. mice) and plant antigens as well as drugs, detergents, metals and immunoenhancers such as isocyanates. Non-antigen specific stimuli that can result in an IgE-mediated reaction include infection, irritants such as smoke, combustion fumes, diesel exhaust particles and sulphur dioxide, exercise, cold and emotional stress. Specific hypersensitivity reactions in atopic and nonatopic individuals with a certain genetic background may result from exposure to proteins in foods (e.g., legumes, peanuts), venom (e.g., insect, snake), vaccines, hormones, antiserum, enzymes, latex, antibiotics, muscle relaxants, vitamins, cytotoxins, opiates, and polysaccharides such as dextrin, iron dextran and polygeline.

Other disorders associated with elevated IgE levels, that appear to be IgE-mediated and are treatable with the formulations of this present invention include: ataxia-telangiectasia, Churg-Strauss Syndrome, eczema, enteritis, gastroenteropathy, graft-versus-host reaction, hyper-IgE (Job's) syndrome, hypersensitivity (e.g., anaphylactic hypersensitivity, candidiasis, vasculitis), IgE myeloma, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis, parasitic diseases (e.g., trypanosomiasis), hypersensitivity vasculitis, urticaria and Wiskott-Aldrich syndrome. Additionally, disorders that may be treatable by lowering IgE levels, regardless of whether the disorders themselves are associated with elevated IgE, and thus should be considered within the scope of “IgE-mediated disorder” include: Addison's disease (chronic adrenocortical insufficiency), alopecia, hereditary angioedema, anigioedema (Bannister's disease, angioneurotic edema), ankylosing spondylitis, aplastic anemia, arteritis, amyloidosis, immune disorders, such as autoimmune hemolytic anemia, autoimmune oophoritis, autoimmune orchitis, autoimmune polyendocrine failure, autoimmune hemolytic anemia, autoimmunocytopenia, autoimmune glomerulonephritis, Behcet's disease, bronchitis, Buerger's disease, bullous pemphigoid, Caplan's syndrome (rheumatoid pneumoconiosis), carditis, celiac sprue, Chediak-Higashi syndrome, chronic obstructive lung Disease (COPD), Cogan-Reese syndrome (iridocorneal endothelial syndrome), CREST syndrome, dermatitis herpetiformis (Duhring's disease), diabetes mellitus, eosinophilic fasciitis, eosinophilic nephritis, episcleritis, extrinsic allergic alveolitis, familial paroxysmal polyserositis, Felty's syndrome, fibrosing alveolitis, glomerulonephritis, Goodpasture's syndrome, granulocytopenia, granuloma, granulomatosis, granuloma myositis, Graves' disease, Guillain-Barre syndrome (polyneuritis), Hashimoto's thyroiditis (lymphadenoid goiter), hemochromatosis, histocytosis, hypereosinophilic syndrome, irritable bowel syndrome, juvenile arthritis, keratitis, leprosy, lupus erythematosus, Lyell's disease, Lyme disease, mixed connective tissue disease, mononeuritis, mononeuritis multiplex, Muckle-Wells syndrome, mucocutaneous lymphoid syndrome (Kawasaki's disease), multicentric reticulohistiocystosis, multiple sclerosis, myasthenia gravis, mycosis fungoides, panninculitis, pemphigoid, pemphigus, pericarditis, polyneuritis, polyarteritis nodoas, psoriasis, psoriatic arthritis, pulmonary arthritis, pulmonary adenomatosis, pulmonary fibrosis, relapsing polychondritis, rheumatic fever, rheumatoid arthritis, rhinosinusitis (sinusitis), sarcoidosis, scleritis, sclerosing cholangitis, serum sickness, Sézary syndrome, Sjögren's syndrome, Stevens-Johnson syndrome, systemic mastocytosis, transplant rejection, thrombocytopenic purpura, thymic alymphoplasia, uveitis, vitiligo, Wegener's granulomatosis.

The term “asthma” refers to a complex disorder characterized by variable and recurring symptoms, reversible airflow obstruction (e.g., by bronchodilator) and bronchial hyperresponsiveness which may or may not be associated with underlying inflammation. Examples of asthma include aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids and other asthmas as mentioned in J Allergy Clin Immunol (2010) 126(5):926-938.

Asthma-Like Symptom includes a symptom selected from the group consisting of shortness of breath, cough (changes in sputum production and/or sputum quality and/or cough frequency), wheezing, chest tightness, bronchioconstriction and nocturnal awakenings ascribed to one of the symptoms above or a combination of these symptoms (Juniper et al (2000) Am. J. Respir. Crit. Care Med., 162(4), 1330-1334).

The term “mild asthma” refers to a patient generally experiencing symptoms or exacerbations less than two times a week, nocturnal symptoms less than two times a month, and is asymptomatic between exacerbations. Mild, intermittent asthma is often treated as needed with the following: inhaled bronchodilators (short-acting inhaled beta2-agonists); avoidance of known triggers; annual influenza vaccination; pneumococcal vaccination every 6 to 10 years, and in some cases, an inhaled beta2-agonist, cromolyn, or nedocromil prior to exposure to identified triggers. If the patient has an increasing need for short-acting beta2-agonist (e.g., uses short-acting beta2-agonist more than three to four times in 1 day for an acute exacerbation or uses more than one canister a month for symptoms), the patient may require a stepup in therapy.

The term “moderate asthma” generally refers to asthma in which the patient experiences exacerbations more than two times a week and the exacerbations affect sleep and activity; the patient has nighttime awakenings due to asthma more than two times a month; the patient has chronic asthma symptoms that require short-acting inhaled beta2-agonist daily or every other day; and the patient's pretreatment baseline PEF or FEV1 is 60 to 80 percent predicted and PEF variability is 20 to 30 percent.

The term “severe asthma” generally refers to asthma in which the patient has almost continuous symptoms, frequent exacerbations, frequent nighttime awakenings due to the asthma, limited activities, PEF or FEV1 baseline less than 60 percent predicted, and PEF variability of 20 to 30 percent.

The term “corticosteroid” includes glucocorticoids and mineralocorticoids. For example, corticosteroid includes, but is not limited to fluticasone (including fluticasone propionate (FP)), beclometasone, budesonide, ciclesonide, mometasone, flunisolide, betamethasone, hydrocortisone, prednisone, prednisolone, methylprednisolone, and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future. Examples of corticosteroids that can be inhaled and are combined with a long-acting beta2-agonist include, but are not limited to: budesonide/formoterol and fluticasone/salmeterol.

A “glucocorticoid” as used herein describes steroidal based agents having anti-inflammatory activity. Glucocorticoids are commonly used to attenuate late phase asthmatic reaction and to treat asthma exacerbations. Example glucocorticoids include prednisone, beclomethasone dipropionate, triamcinolone acetonide, flunisolide, betamethasone, budesonide, dexamethasone, desamehasone tramcinolone, fludrocortisone acetate, flunisolide, fluticasone propionate, hydrocortisone, prednisolone [including methylprednisolone (e.g., SOLU-MEDROL® methlprednisolone sodium succinate)], and triamcinolone.

The term “FEV1” refers to the volume of air exhaled in the first second of a forced expiration. It is a measure of airway obstruction. Provocative concentration of methacholine required to induce a 20% decline in FEV1 (PC20) is a measure of airway hyper-responsiveness. FEV1 may be noted in other similar ways, e.g., FEV₁, and it should be understood that all such similar variations have the same meaning.

The term “relative change in FEV1”=(FEV1 at week 12 of treatment−FEV1 prior to start of treatment) divided by FEV1.

An “autoimmune disorder” herein is a disease or disorder arising from and directed against an individual's own tissues or organs or a co-segregation or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to any one theory regarding B-cell mediated autoimmune disorder, it is believed that B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of these pathways may participate to different degrees in the pathology of autoimmune diseases.

“Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems (for example, systemic lupus erythematosus (SLE), rheumatoid arthritis (RA), polymyositis, etc.). Preferred such diseases include autoimmune rheumatologic disorders (such as, for example, RA, Sjögren's syndrome, scleroderma, lupus such as SLE and lupus nephritis, polymyositis-dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, RA, ulcerative colitis, ANCA-associated vasculitis, lupus, multiple sclerosis, Sjögren's syndrome, Graves' disease, IDDM, pernicious anemia, thyroiditis, and glomerulonephritis.

The term “cytotoxic agent” as used herein refers to a substance that inhibits or prevents the function of cells and/or causes destruction of cells. The term includes radioactive isotopes (e.g. At²¹¹, I¹³¹, I¹²⁵, Y⁹⁰, Re¹⁸⁶, Re¹⁸⁸, Sm¹⁵³, Bi²¹², P32 and radioactive isotopes of Lu), and toxins such as small molecule toxins or enzymatically active toxins of bacterial, fungal, plant or animal origin, or fragments thereof.

The term “cytokine” is a generic term for proteins released by one cell population that act on another cell as intercellular mediators. Examples of such cytokines are lymphokines, monokines; interleukins (ILs) such as IL-1, IL-1α, IL-2, IL-3, IL-4, IL-5, IL-6, IL-7, IL-8, IL-9, IL-11, IL-12, IL-13, IL-15, including PROLEUKIN® rIL-2; a tumor-necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; estradiol; hormone-replacement therapy; androgens such as calusterone, dromostanolone propionate, epitiostanol, mepitiostane, or testolactone; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, gonadotropin-releasing hormone; inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-C SF); granulocyte-macrophage-CSF (GM-CSF); and granulocyte-C SF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

A “TNF antagonist” is defined herein as a molecule that decreases, blocks, inhibits, abrogates, or interferes with TNFα activity in vitro, in situ, and/or preferably in vivo. A suitable TNF antagonist can also decrease block, abrogate, interfere, prevent and/or inhibit TNF RNA, DNA or protein synthesis, TNFα release, TNFα receptor signaling, membrane TNFα cleavage, TNFα activity, TNFα production and/or synthesis. Such TNF antagonists include, but are not limited to, anti-TNFα antibodies, antigen-binding fragments thereof, specified mutants or domains thereof that bind specifically to TNFα that, upon binding to TNFα, destroy or deplete cells expressing the TNFα in a mammal and/or interferes with one or more functions of those cells, a soluble TNF receptor (e.g., p55, p70 or p85) or fragment, fusion polypeptides thereof, a small-molecule TNF antagonist, e.g., TNF binding protein I or II (TBP-I or TBP-II), nerelimonmab, CDP-571, infliximab, enteracept (ENBREL™), adalimulab (HUMIRA™), CDP-571, CDP-870, afelimomab, lenercept, and the like), antigen-binding fragments thereof, and receptor molecules that bind specifically to TNFα; compounds that prevent and/or inhibit TNFα synthesis, TNFα release or its action on target cells, such as thalidomide, tenidap, phosphodiesterase inhibitors (e.g, pentoxifylline and rolipram), A2b adenosine receptor agonists and A2b adenosine receptor enhancers; compounds that prevent and/or inhibit TNFα receptor signalling, such as mitogen activated protein (MAP) kinase inhibitors; compounds that block and/or inhibit membrane TNFα cleavage, such as metalloproteinase inhibitors; compounds that block and/or inhibit TNFα activity, such as angiotensin converting enzyme (ACE) inhibitors (e.g., captopril); and compounds that block and/or inhibit TNFα production and/or synthesis, such as MAP kinase inhibitors. The preferred antagonist comprises an antibody.

“Tumor necrosis factor-alpha”, TNF-alpha”, or “TNFα” refers to a human TNFα molecule comprising the amino acid sequence of Pennica et al., Nature, 312:721 (1984) or Aggarwal et al., JBC, 260:2345 (1985). A “TNFα inhibitor” herein is an agent that inhibits, to some extent, a biological function of TNFα, generally through binding to TNFα and neutralizing its activity. Examples of TNFα inhibitors herein include etanercept (ENBREL®), infliximab (REMICADE®), and adalimumab (HUMIRAT™).

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (U52002/0042368), antibodies to alphavbeta3 integrin (EP 633945), aza-bridged bicyclic amino acid derivatives (WO 2002/02556), etc.

The term “immunosuppressive agent” refers to a substance that acts to suppress or mask the immune system of the subject being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); trocade (Ro32-355); a peripheral sigma receptor antagonist such as ISR-31747; alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, rimexolone, and dexamethasone; dihydrofolate reductase inhibitors such as methotrexate (oral or subcutaneous); anti-malarial agents such as chloroquine and hydroxychloroquine; sulfasalazine; leflunomide; cytokine release inhibitors such as SB-210396 and SB-217969 monoclonal antibodies and a MHC II antagonist such as ZD2315; a PG1 receptor antagonist such as ZD4953; a VLA4 adhesion blocker such as ZD7349; anti-cytokine or anti-cytokine receptor antibodies including anti-interferon-alpha, -beta, or -gamma antibodies, anti-TNF-α antibodies (infliximab (REMICADE®) or adalimumab), anti-TNF-α immunoadhesin (etanercept), anti-TNF-beta antibodies, interleukin-1 (IL-1) blockers such as recombinant HuIL-1Ra and IL-1B inhibitor, anti-interleukin-2 (IL-2) antibodies and anti-IL-2 receptor antibodies; IL-2 fusion toxin; anti-L3T4 antibodies; leflunomide; heterologous anti-lymphocyte globulin; OPC-14597; NISV (immune response modifier); an essential fatty acid such as gammalinolenic acid or eicosapentaenoic acid; CD-4 blockers, pan-T antibodies, preferably anti-CD3 or anti-CD4/CD4a antibodies; co-stimulatory modifier (e.g., CTLA4-Fc fusion, also known as ABATACEPT™; anti-interleukin-6 (IL-6) receptor antibodies and antagonists; anti-LFA-1 antibodies, including anti-CD11a and anti-CD18 antibodies; soluble peptide containing a LFA-3 binding domain (WO 1990/08187); streptokinase; IL-10; anti-IL-4 antagonists, anti-IL-13 antagonists and bispecific anti-IL-4/IL-13 antagonist antibodies, transforming growth factor-beta (TGF-beta); streptodornase; RNA or DNA from the host; FK506; RS-61443; enlimomab; CDP-855; PNP inhibitor; CH-3298; GW353430; 4162W94, chlorambucil; deoxyspergualin; rapamycin; T-cell receptor (U.S. Pat. No. 5,114,721); T-cell receptor fragments (Offner et al., Science, 251: 430-2 (1991); WO 1990/11294; Janeway, Nature, 341: 482-483 (1989); and WO 1991/01133); BAFF antagonists such as BAFF antibodies and BR3 antibodies; zTNF4 antagonists (Mackay and Mackay, Trends Immunol., 23:113-5 (2002)); biologic agents that interfere with T-cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate (MTX).

“Disease-modifying anti-rheumatic drugs” or “DMARDs” include, e.g., chloroquine, hydroxycloroquine, myocrisin, auranofin, sulfasalazine, methotrexate, leflunomide, etanercept, infliximab (and oral and subcutaneous MTX), azathioprine, D-penicilamine, gold salts (oral), gold salts (intramuscular), minocycline, cyclosporine, e.g., cyclosporine A and topical cyclosporine, staphylococcal protein A (Goodyear and Silverman, J. Exp. Med., 197:1125-39 (2003)), including salts and derivatives thereof, etc.

A “B cell” is a lymphocyte that matures within the bone marrow, and includes a naive B cell, memory B cell, or effector B cell (plasma cells). The B cell herein may be normal or non-malignant.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The preferred B-cell surface marker is preferentially expressed on B cells compared to other non-B-cell tissues of a mammal and may be expressed on both precursor and mature B cells. The most preferred such markers are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al., Proc. Natl. Acad. Sci. (USA) 82:1766 (1985), for example.

The “CD22” antigen, or “CD22,” also known as BL-CAM or Lyb8, is a type 1 integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, for example, in Wilson et al., J. Exp. Med. 173:137 (1991) and Wilson et al., J. Immunol. 150:5013 (1993).

An “antibody that binds to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g. via apoptosis).

Examples of CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework patched” or humanized 1F5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HUMAX-CD20™ fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO04/035607 (Teeling et al.); AME-133™ antibodies (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are chimeric, humanized, or human CD20 antibodies, more preferably rituximab, a humanized 2H7, chimeric or humanized A20 antibody (Immunomedics), and HUMAX-CD20™ human CD20 antibody (Genmab).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof that retain the ability to bind CD20. Purely for the purposes herein and unless indicated otherwise, a “humanized 2H7” refers to a humanized CD20 antibody, or an antigen-binding fragment thereof, wherein the antibody is effective to deplete primate B cells in vivo. The antibody includes those set forth in US 2006/0062787 and the figures thereof, and including version 114, the sequences of which are provided in US 2006/0188495. See also US 2006/0034835 and US 2006/0024300. In a summary of various preferred embodiments of the invention, the V region of variants based on 2H7 version 16 as disclosed in US 2006/0062787 will have the amino acid sequences of v16 except at the positions of amino acid substitutions that are indicated in the table below. Unless otherwise indicated, the 2H7 variants will have the same L chain as that of v16.

| H7 | Heavy chain | Light chain | |
|---|---|---|---|
| version | (V_{H}) changes | (V_{L}) changes | Fc changes |
| 16 | | | — |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K334A, K322A |
| 138 | D56A, N100A | M32L, S92A | S298A, E333A, K334A, K326A |
| 477 | D56A, N100A | M32L, S92A | S298A, E333A, K334A, |
| | | | K326A, N434W |
| 375 | — | — | K334L |

One preferred humanized 2H7 is an intact antibody or antibody fragment having the sequence of version 16. Another preferred humanized 2H7 has the sequences of version 114.

“BAFF antagonists” are any molecules that block the activity of BAFF or BR3. They include immunoadhesins comprising a portion of BR3, TACI or BCMA that binds BAFF, or variants thereof that bind BAFF. In other aspects, the BAFF antagonist is a BAFF antibody. A “BAFF antibody” is an antibody that binds BAFF, and preferably binds BAFF within a region of human BAFF comprising residues 162-275 of human BAFF. In another aspect, the BAFF antagonist is a BR3 antibody. A “BR3 antibody” is an antibody that binds BR3, and preferably binds BR3 within a region of human BR3 comprising residues 23-38 of human BR3. The sequences of human BAFF and human BR3 are found, e.g., in US 2006/0062787. Other examples of BAFF-binding polypeptides or BAFF antibodies can be found in, e.g., WO 2002/092620, WO 2003/014294, Gordon et al., Biochemistry 42(20):5977-83 (2003), Kelley et al., J. Biol. Chem. 279:16727-35 (2004), WO 1998/18921, WO 2001/12812, WO 2000/68378 and WO 2000/40716.

“Anti-IgE antibody” includes any antibody that binds specifically to IgE in a manner so as to not induce cross-linking when IgE is bound to the high affinity receptor on mast cells and basophils. Exemplary antibodies include the antibodies of the invention as well as rhuMabE25 (E25, XOLAIR®), E26, E27, as well as CGP-5101 (Hu-901) and the HA antibody. The amino acid sequences of the heavy and light chain variable domains of the humanized anti-IgE antibodies E25, E26 and E27 are disclosed, for example in U.S. Pat. No. 6,172,213 and WO99/01556. The CGP-5101 (Hu-901) antibody is described in Corne et al., (1997) J. Clin. Invest. 99(5): 879-887, WO 92/17207 and ATCC Dep. Nos. BRL-10706, BRL-11130, BRL-11131, BRL-11132 and BRL-11133. The HA antibody is described in U.S. Ser. No. 60/444,229, WO2004/070011 and WO2004/070010.

The term “about” as used herein refers to the usual error range for the respective value readily known to the skilled person in this technical field. Reference to “about” a value or parameter herein includes (ad describes) embodiments that are directed to that value or parameter per se. As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the invention described herein include “comprising,” “consisting,” and “consisting essentially of” aspects and embodiments.

### III. Compositions and Methods of the Invention

Provided herein are anti-IgE antibodies that bind to the M1′ segment of an IgE and method of using the anti-IgE antibodies for treating or preventing an IgE-mediated disorder.

With respect to all methods described herein, reference to an anti-IgE/M′ antibody also includes compositions comprising one or more of those agents. Such compositions may further comprise suitable excipients, such as pharmaceutically acceptable excipients (carriers) including buffers, acids, bases, sugars, diluents, preservatives, and the like, which are well known in the art and are described herein. The present methods can be used alone or in combination with other conventional methods of treatment.

A. Anti-IgE Antibodies

The antibodies that may be used in the methods described herein include anti-IgE antibodies that bind the M1′ segment of IgE. The anti-IgE antibodies described herein has one or more of the following characteristics: (a) specifically binds to the M1′ segment of IgE (such as a human IgE); (b) induces apoptosis of IgE-expressing B cells in vitro and/or in vivo; (c) depletes IgE-M1′ expressing cells (such as IgE-switched B cells, IgE plasmablast, and IgE memory B cells) via apoptosis and/or antibody-dependent cell-mediated cytotoxicity in vitro; (d) depletes IgE-M1′ expressing cells in a mammal; (e) reduces serum total IgE in a mammal; (f) reduces allergen-specific IgE in a mammal; (g) prevents or reduces an allergen-induced increase in serum total IgE or allergen-specific IgE in a mammal; (h) induces calcium flux in IgE-expressing B cells in vitro and/or in vivo; and (i) treats and/or prevents an IgE-mediated disorder (e.g., allergic rhinitis, and allergic asthma).

Methods for measuring the depletion of IgE-M1′ expressing cells (such as IgE-switched B cells, IgE plasmablast, and IgE memory B cells) are known in the art and described in U.S. Pat. No. 8,071,097. M1′ expressing B cells can be detected by quantitative PCR in human peripheral blood. In brief, RNA is extracted from whole blood collected in PaxGene collection tubes. RNA is converted to cDNA and reverse (GTGGCAGAGCACCCTATCC) (SEQ ID NO:41) and forward (CAGCGAGCGGTGTCTGT) (SEQ ID NO:42) primers, and fluorescent probe (CCAGCCCGGGATTT) (SEQ ID NO:43) are using to amplify M1′ mRNA by TaqMan. M1′expressing B cells can also be detected by flow cytometry in human peripheral blood. Briefly, B cells are enriched from approximately 40-50 ml of whole blood using RossetteSep kit. The enriched B cells are subsequently stained for surface markers of memory B cells and M1′. Methods (such as ELISA) known in the art may be used to measure the level of serum total IgE and allergen-specific IgE. Standard clinical assays for total and allergen specific IgE are Siemens Immulite 2000 assays (Siemens Medical Solutions Diagnostics, Los Angeles Calif.) and Phadia ImmunoCAP assays (Phadia Inc.). See Li, et al., Ann Clin Lab Sci., 34(1):67-74 (2004) and Libeer, et al., Clin Chem Lab Med., 45(3):413-415.

Methods for measuring calcium flux in IgE-expressing B cells induced by anti-IgE antibodies are known in the art. See, e.g., U.S. Pat. No. 8,071,097, Example 7.

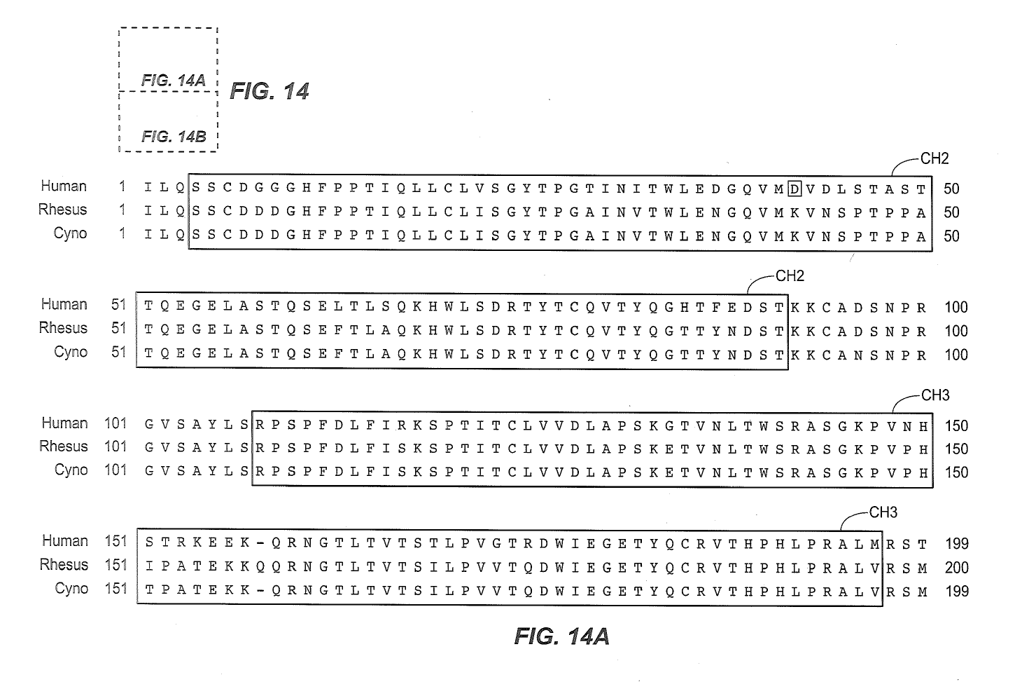

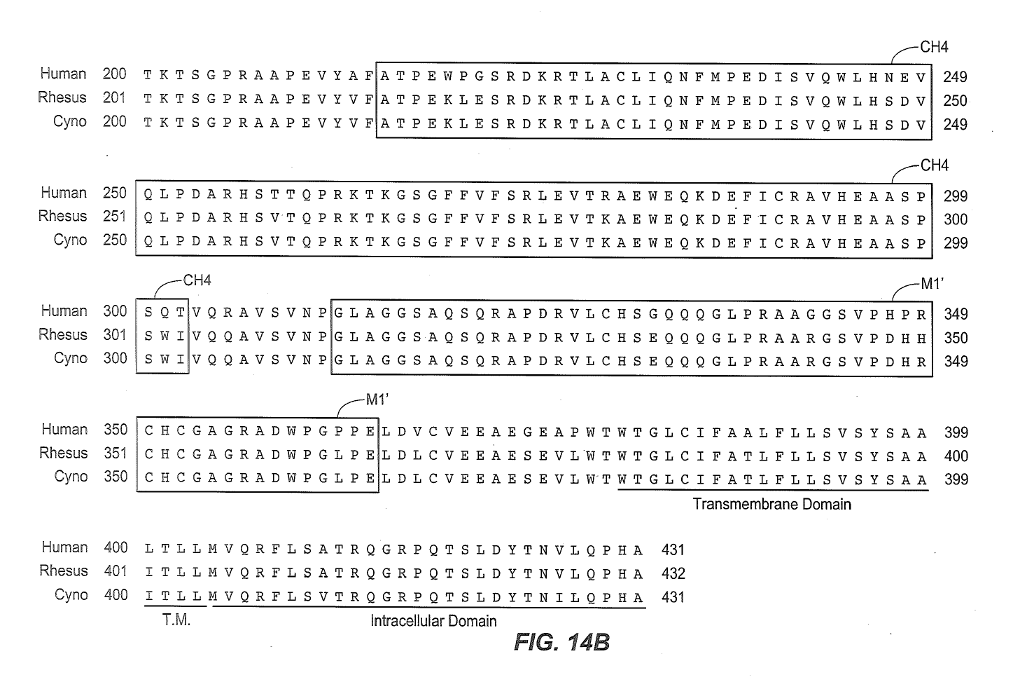

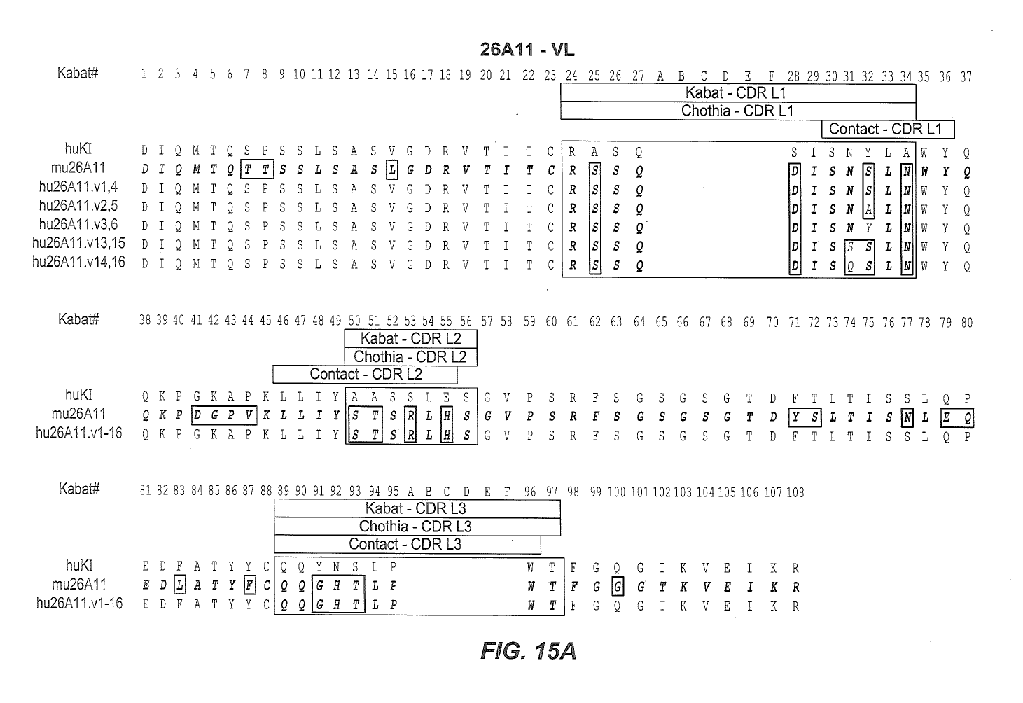

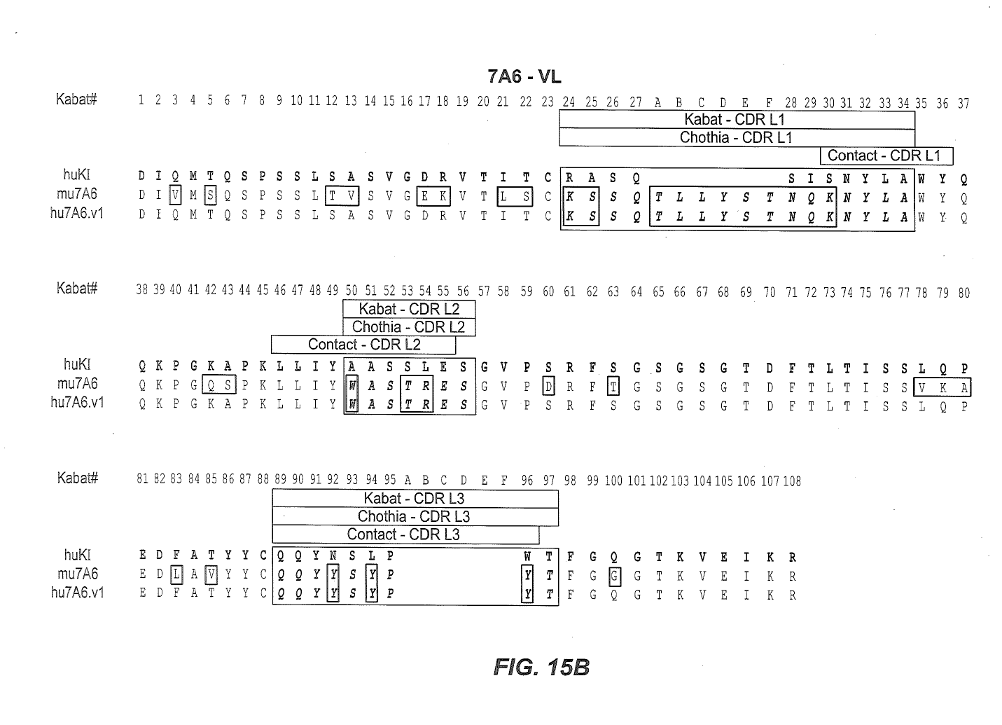

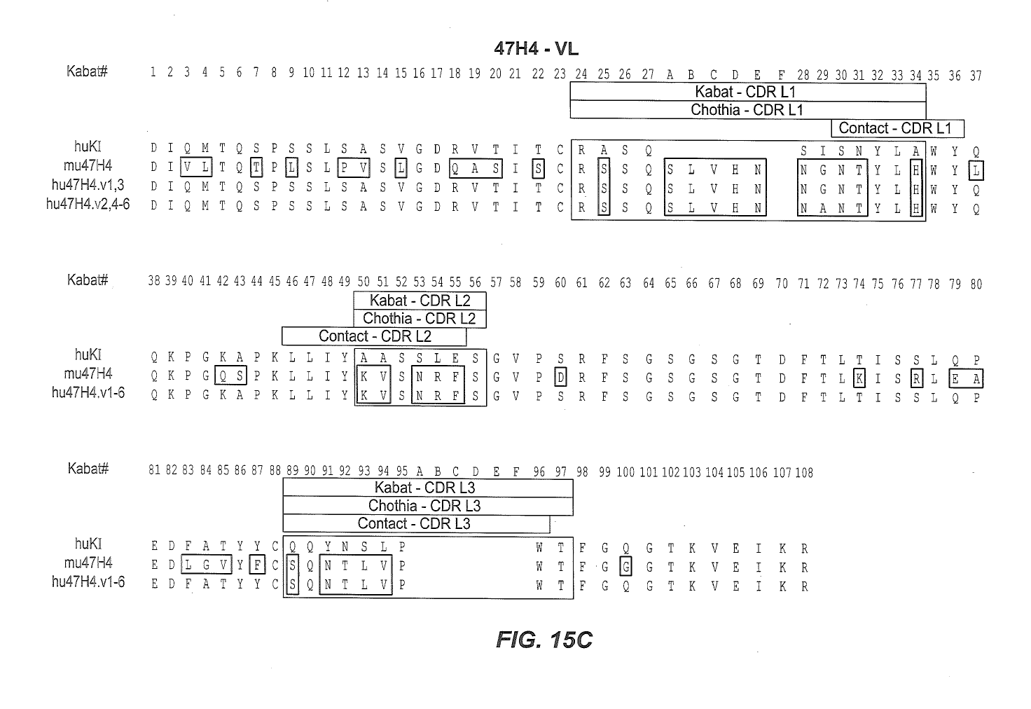

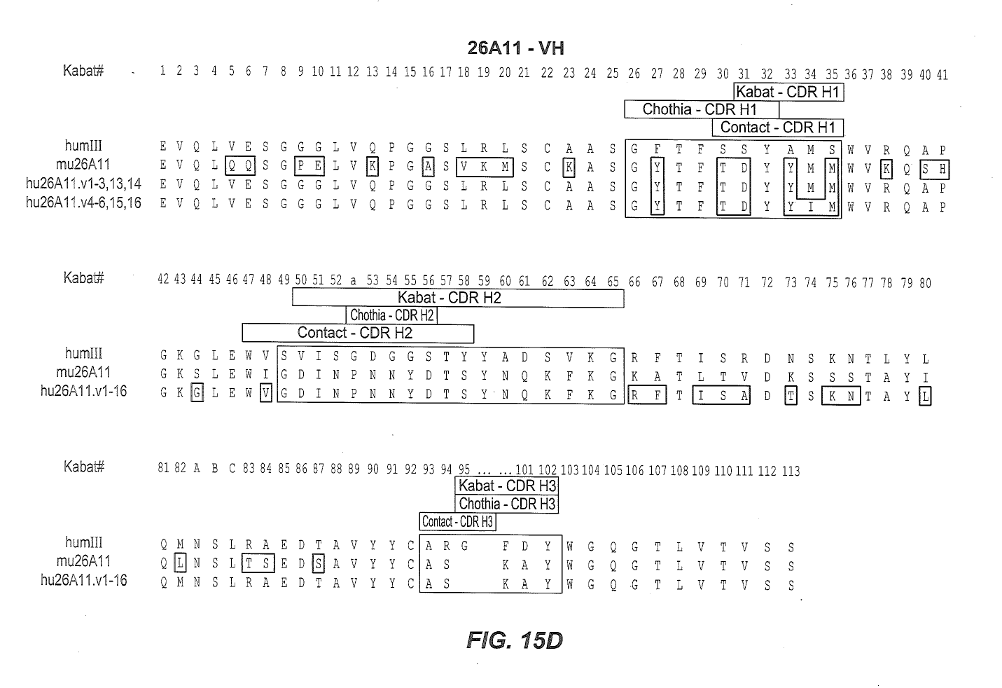

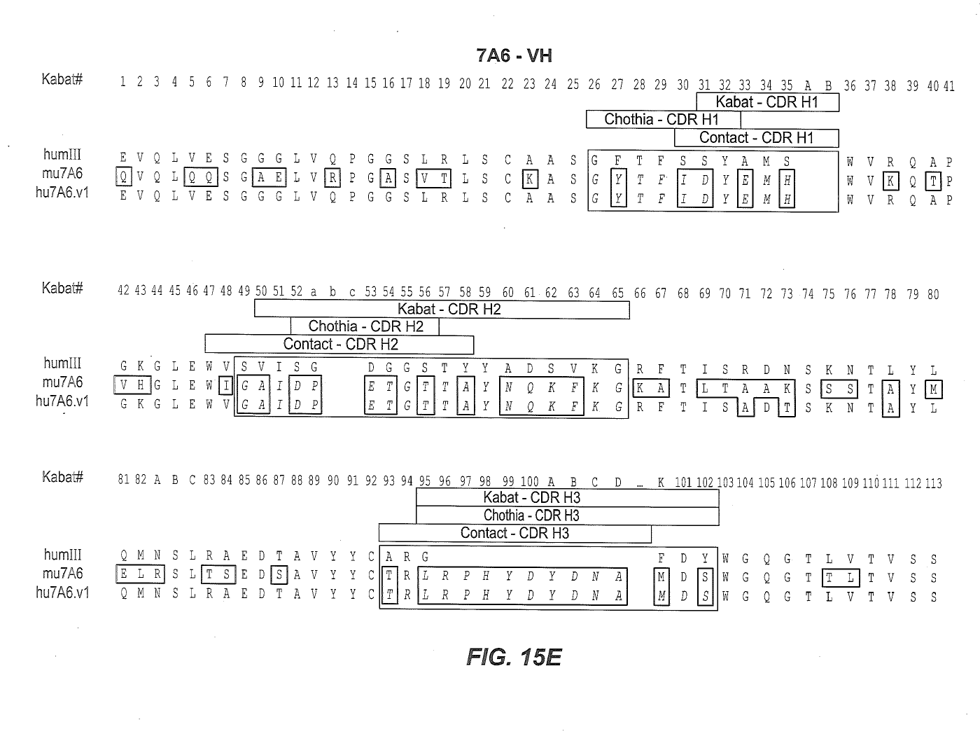

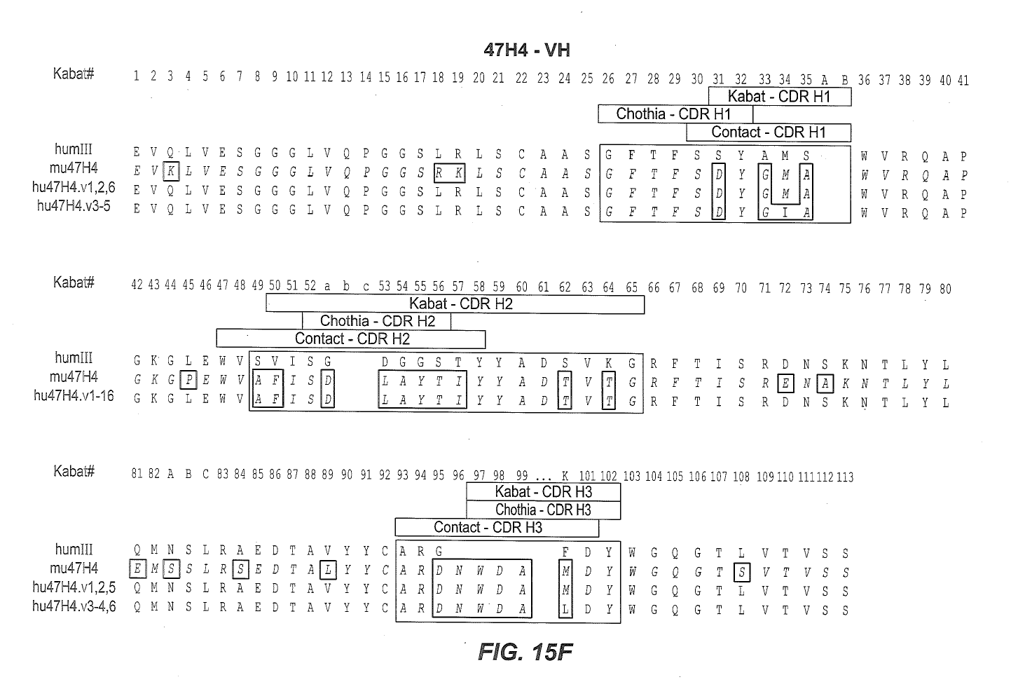

In some embodiments, the anti-IgE antibody binds to any epitope within the M1′ segment of human IgE, rhesus IgE, and/or Cyno IgE shown in FIG. 14. In some embodiments, the antibody specifically binds to the same epitope as the one bound by an antibody selected from the group consisting of: 47H4, 7A6, 26A11, 47H4v1, 47H4v2, 47H4v3, 47H4v4, 47H4v5, 47H4v6, 7A6v1, and 26A11v6. These antibodies are described in U.S. Pat. No. 8,071,097. The heavy and light chain variable amino acid sequences of these antibodies are shown in FIGS. 15A-15F. In some embodiments, the antibody binds to an epitope corresponding to a peptide selected from the group consisting of: SAQSQRAPDRVLCHS (SEQ ID NO:4), RAPDRVLCHSGQQQG (SEQ ID NO:5), GQQQGLPRAAGGSVP (SEQ ID NO:6) or PRAAGGSVPHPRCH (SEQ ID NO:7). In some embodiments, the antibody is an antigen-binding fragment. In some embodiments, the antibody is a humanized antibody, a human antibody, or a chimeric antibody. In some embodiments, the antibody is afucosylated.

In some embodiments, the anti-IgE antibody comprises the heavy chain and light chain HVRs (such as one, two, three, four, five or six HVRs) of the antibody shown in FIGS. 15A-15F. In some embodiments, the anti-IgE antibody comprises a light chain comprising HVR1, HVR2 and HVR3 (such as the three Kabat CDRs, Chothia CDRs, or contact CDRs) of the light chain of the antibody shown in FIGS. 15A-15C, and/or a heavy chain comprising HVR1, HVR2 and HVR3 (such as the three Kabat CDRs, Chothia CDRs, or contact CDRs) of the heavy chain of the antibody shown in FIGS. 15D-15F. In some embodiments, the antibody comprises the heavy and light chain variable region amino acid sequences of the antibodies shown in FIGS. 15A-15F. In some embodiments, the antibody comprises the heavy and light chain HVRs of antibody 47H4v5. In some embodiments, the antibody comprises the heavy and light chain amino acid sequences of antibody 47H4v5. In some embodiments, the antibody is an antibody selected from the consisting of 26A11 v1-16, 7A6v1, and 47H4v1-6. In some embodiments, the antibody is afucosylated.

In some embodiments, the antibody is antibody 47H4v5 or an antigen-binding fragment thereof. Antibody 47H4v5 (MEMP1972A) have the heavy chain amino acid sequence of SEQ ID NO:39 and the light chain amino acid sequence of SEQ ID NO:40. In some embodiments, the antibody is afucosylated.

47H4v5 heavy chain
(SEQ ID NO: 39)
EVQLVESGGGLVQPGGSLRLSCAASGFTFSDYGIAWVRQAPGKGLEWVAF
ISDLAYTIYYADTVTGRFTISRDNSKNTLYLQMNSLRAEDTAVYYCARDN
WDAMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
47H4v5 light chain
(SEQ ID NO: 40)
DIQMTQSPSSLSASVGDRVTITCRSSQSLVHNNANTYLHWYQQKPGKAPK
LLIYKVSNRFSGVPSRFSGSGSGTDFTLTISSLQPEDFATYYCSQNTLVP
WTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In some embodiments, the anti-IgE antibodies described herein bind to an M1′ segment of human IgE with a Scatchard binding affinity to human IgE that is equivalent to that of the murine anti-IgE antibody 47H4 or a humanized variant thereof (such as 47H4v1-6). The Scatchard binding affinity is measure as described in Example 2A of U.S. Pat. No. 8,071,097. In some embodiments, the affinity is equivalent to the binding affinity of 47H4. In some embodiments, the affinity is between 0.30 and 0.83 nM. In yet another specific aspect, the affinity is equivalent to the binding affinity of 47H4v5. In a further specific aspect, the affinity is about 1.5 nM.

B. Antibody Preparation

The antibodies useful in the present invention can encompass monoclonal antibodies, polyclonal antibodies, antibody fragments (e.g., Fab, Fab′-SH, Fv, scFv, and F(ab′)₂), chimeric antibodies, bispecific antibodies, multivalent antibodies, heteroconjugate antibodies, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or of any other origin (including chimeric or humanized antibodies).

### 1) Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysien residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the antigen, immunogenic conjugates, or derivatives by combining, e.g., 100 μg or 5 μg or the protein or conjugate (for rabbits or mice, respectively) with 3 volumes of Freund's complete adjuvant and injecting the solution intradermally at multiple sites. One month later, the animals are boosted with 1/5 to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### 2) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally either peripheral blood lymphocytes (“PBLs”) are used if cells of human origin are desired, or spleen cells or lymph node cells are used if non-human mammalian sources are desired. The lymphoctyes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies. The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### 3) Humanized Antibodies.

The antibodies of the invention may further comprise humanized or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domain, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Various forms of the humanized antibody are contemplated. For example, the humanized antibody may be an antibody fragment, such as an Fab, which is optionally conjugated with one or more cytotoxic agent(s) in order to generate an immunoconjugate. Alternatively, the humanized antibody may be an intact antibody, such as an intact IgG1 antibody.

### 4) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); U.S. Pat. Nos. 5,591,669 and WO 97/17852.

Alternatively, phage display technology can be used to produce human antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905. The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human antibodies may also be generated in vitro by activated B cells (see U.S. Pat. Nos. 5,567,610 and 5,229,275).

### 5) Antibody Fragments

In certain circumstances there are advantages to using antibody fragments, rather than whole antibodies. Smaller fragment sizes allow for rapid clearance, and may lead to improved access to solid tumors.

Various techniques have been developed for the production of antibody fragments. Traditionally, these fragments were derived via proteolytic digestion of intact antibodies (see, e.g., Morimoto et al., J Biochem Biophys. Method. 24:107-117 (1992); and Brennan et al., Science 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ with increase in vivo half-life is described in U.S. Pat. No. 5,869,046. In other embodiments, the antibody of choice is a single chain Fv fragment (scFv). See WO 93/16185; U.S. Pat. No. 5,571,894 and U.S. Pat. No. 5,587,458. The antibody fragment may also be a “linear antibody”, e.g., as described in U.S. Pat. No. 5,641,870. Such linear antibody fragments may be monospecific or bispecific.

### 6) Antibody Dependent Enzyme-Mediated Prodrug Therapy (ADEPT)

The antibodies of the present invention may also be used in ADEPT by conjugating the antibody to a prodrug-activating enzyme which converts a prodrug (e.g. a peptidyl chemotherapeutic agent, see WO 81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to convert it into its more active, cytotoxic form.

Enzymes that are useful in the method of this invention include, but are not limited to, glycosidase, glucose oxidase, human lysozyme, human glucuronidase, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug 5-fluorouracil; proteases, such as serratia protease, thermolysin, subtilisin, carboxypeptidases (e.g., carboxypeptidase G2 and carboxypeptidase A) and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin Vamidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes” can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The above enzymes can be covalently bound to the polypeptide or antibodies described herein by techniques well known in the art such as the use of the heterobifunctional cross-linking agents discussed above. Alternatively, fusion proteins comprising at least the antigen binding region of the antibody of the invention linked to at least a functionally active portion of an enzyme of the invention can be constructed using recombinant DNA techniques well known in the art (see, e.g. Neuberger et al., Nature 312: 604-608 (1984)).

### 7) Bispecific and Polyspecific Antibodies

Bispecific antibodies (BsAbs) are antibodies that have binding specificities for at least two different epitopes, including those on the same or another protein. Alternatively, one arm can be armed to bind to the target antigen, and another arm can be combined with an arm that binds to a triggering molecule on a leukocyte such as a T-cell receptor molecule (e.g., CD3), or Fc receptors for IgG (FcγR) such as FcγR1 (CD64), FcγRII (CD32) and FcγRIII (CD16), so as to focus and localize cellular defense mechanisms to the target antigen-expressing cell. Such antibodies can be derived from full length antibodies or antibody fragments (e.g. F(ab′)₂ bispecific antibodies).

Bispecific antibodies may also be used to localize cytotoxic agents to cells which express the target antigen. Such antibodies possess one arm that binds the desired antigen and another arm that binds the cytotoxic agent (e.g., saporin, anti-interferon-α, vinca alkoloid, ricin A chain, methotrexate or radioactive isotope hapten). Examples of known bispecific antibodies include anti-ErbB2/anti-FcgRIII (WO 96/16673), anti-ErbB2/anti-FcgRI (U.S. Pat. No. 5,837,234), anti-ErbB2/anti-CD3 (U.S. Pat. No. 5,821,337).

Methods for making bispecific antibodies are known in the art. Traditional production of full length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecules provides for an easy way of separation. This approach is disclosed in WO 94/04690. For further details of generating bispecific antibodies, see, for example, Suresh et al., Methods in Enzymology 121: 210 (1986). According to another approach described in WO 96/27011 or U.S. Pat. No. 5,731,168, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell culture have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Natl. Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147: 60 (1991).

Exemplary bispecific antibodies may bind to two different epitopes on a given molecule. Alternatively, an anti-protein arm may be combined with an arm which binds to a triggering molecule on a leukocyte such as a T-cell receptor molecule (e.g., CD2, CD3, CD28 or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF).

### 8) Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### 9) Heteroconjugate Antibodies

Heteroconjugate antibodies are also within the scope of the present invention. Heteroconjugate antibodies are composed of two covalently joined antibodies. For example, one of the antibodies in the heteroconjugate can be coupled to avidin, the other to biotin. Such antibodies have, for example, been proposed to target immune system cells to unwanted cells, U.S. Pat. No. 4,676,980, and for treatment of HIV infection. WO 91/00360, WO 92/200373 and EP 0308936. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Heteroconjugate antibodies may be made using any convenient cross-linking methods. Suitable cross-linking agents are well known in the art, and are disclosed in U.S. Pat. No. 4,676,980, along with a number of cross-linking techniques.

### 10) Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG₂, IgG₃, or IgG₄) that is responsible for increasing the in vivo serum half-life of the IgG molecule.

### 11) Immunoconjugates

The invention also pertains to immunoconjugates or antibody-drug conjugates (ADC), comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate). Such ADC must show an acceptable safety profile. The use of ADCs for the local delivery of cytotoxic or cytostatic agents, e.g., drugs to kill or inhibit tumor cells in the treatment of cancer [Syrigos and Epenetos, Anticancer Research 19: 605-14 (1999); Niculeascu-Duvaz and Springer, Adv. Drug Del. Rev. 26: 151-72 (1997); U.S. Pat. No. 4,975,278] theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., Lancet, 603-05 (1986); Thorpe, (1985) Antibody Carriers Of Cytotoxic Agents In Cancer Therapy: A Review, in Monoclonal Antibodies '84: Biological And Clinical Applications, A. Pinchera et al. (eds), pp. 475-506). Maximal efficacy with minimal toxicity is sought thereby. Both polyclonal antibodies and monoclonal antibodies have been reported as useful in these strategies (Rowland et al., Cancer Immunol. Immunother. 21:183-87 (1986)). Drugs used in these methods include daunomycin, doxorubicin, methotrexate, and vindesine. Toxins used in antibody-toxin conjugates include bacterial toxins such as diphtheria toxin, plant toxins such as ricin, small molecule toxins such as geldanamycin (Mandler et al., J. Nat. Cancer Inst. 92(19):1573-81 (2000); Mandler et al., Bioorganic & Med. Chem. Letters 10:1025-28 (2000); Mandler et al., Bioconjugate Chem. 13: 786-91 (2002)), maytansinoids (EP 1391213; Liu et al., Proc. Natl. Acad. Sci. USA 93: 8618-23 (1996)), and calicheamicin (Lode et al., Cancer Res. 58:2928 (1998); and Hinman et al., Cancer Res. 53:3336-42 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Chemotherapeutic agents useful in the generation of such immunoconjugates have been described above, and include BCNU, streptozoicin, vincristine, vinblastine, adriamycin and 5-fluorouracil. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triamine-pentaacetic acid (MX-DTPA) is an exemplary chelating agent for conjugation of radionucleotide to the antibody. See, for example, WO 1994/11026.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al., Cancer Res. 52:127-131 (1992)) may be used.

Additionally, the small molecule toxins such as calicheamicin, maytansine (U.S. Pat. No. 5,208,020), trichothene and CC1065 are also contemplated as conjugatable toxins for use with the inventive formulation. In one embodiment, the full length antibody or antigen binding fragments thereof can be conjugated to one or more maytansinoid molecules (e.g., about 1 to about 10 maytansinoid molecules per antibody molecule). Maytansinoids are mitotic inhibitors which act by inhibiting tubulin polymerization. Maytansinoids, isolated from natural sources or prepared synthetically, including maytansine, maytansinal and derivatives and analogues thereof have been described, see e.g., U.S. Pat. No. 5,208,020 and references cited therein (see col. 2, line 53 to col. 3, line 10) and U.S. Pat. Nos. 3,896,111 and 4,151,042. Methods of preparing antibody-maytansinoid conjugates are also described in U.S. Pat. No. 5,208,020. In a preferred embodiment, a maytansinoid is linked to the antibody via a disulfide or other sulfur-containing linker group. Maytansine may, for example, be converted to May-SS-Me, which may be reduced to May-SH3 and reacted with modified antibody to generate a maytansinoid-antibody immunoconjugate. Chari et al., Cancer Res. 52: 127-131 (1992). The antibody can be modified by known methods and the antibody containing free or protected thiol groups is then reacted with a disulfide containing maytansinoid to produce the conjugate. The cytotoxicity of the antibody-maytansinoid conjugate can be measured in vitro or in vivo by known methods and the IC₅₀ determined.

Calicheamicin is another immunoconjugate of interest. The calicheamicin family of antibiotics are capable of producing double-stranded DNA breaks at sub-picomolar concentrations. Structural analogues of calicheamicin which may be used include, but are not limited to, γ₁¹, α₂¹, α₃¹, N-aceytl-γ₁¹, PSAG and θ¹₁ (Hinman et al., Cancer Res. 53:3336-3342 (1993) and Lode et al., Cancer Res. 58:2925-2928 (1998)). Other anti-tumor drugs that the antibody can be conjugated to include QFA which is an antifolate. Both calicheamicin and QFA have intracellular sites of actions and do not readily cross the plasma membrane. Therefore, cellular uptake of these agents through antibody mediated internalization greatly enhances their cytotoxic effects.

Immunoconjugates formed between an antibody and a compound with nucleolytic activity (e.g., a ribonuclease or DNA endonuclease such as deoxyribonuclease, DNase) are also contemplated. In the ADCs of the invention, an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody.

Ab-(L-D)ₚ Formula I

Nucleophilic groups on antibodies include, but are not limited to: (i) N-terminal amine groups, (ii) side-chain amine groups, e.g. lysine, (iii) side-chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

ADCs of the invention may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups that may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Domen et al., J. Chromatog., 510: 293-302 (1990)). In another embodiment, proteins containing N-terminal serine or threonine residues can react with sodium meta-periodate, resulting in production of an aldehyde in place of the first amino acid (Geoghegan and Stroh, Bioconjugate Chem. 3:138-46 (1992); U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide that does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

The ADCs herein are optionally prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), which are commercially available (e.g., Pierce Biotechnology, Inc., Rockford, Ill.).

The antibody may also be conjugated to a highly radioactive atom. A variety of radionuclides are available for the production of radioconjugated antibodies. Examples include At²¹¹, Bi²¹², I¹³¹, In¹³¹, Y⁹⁰, Re¹⁸⁶, Re¹⁸⁸, Sm¹⁵³, P³² and Pb²¹² and radioactive isotopes of Lu. When the conjugate is used for diagnosis, it may comprise a radioactive atom for scintigraphic studies, for example Tc⁹⁹ or I¹²³, or a spin label for nuclear magnetic resonance (nmr) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such Tc⁹⁹ or I¹²³, Re¹⁸⁶, Re¹⁸⁸ and In¹¹¹ can be attached via a cysteine residue in the peptide. Yttrium-90 can be attached via a lysine residue. The IODOGEN® method can be used to incorporate iodine-123, Fraker et al., Biohem. Biophys. Res. Commun. 80:49-57 (1978). Other methods of conjugating radionuclides are described in “Monoclonal Antibodies in Immunoscintigraphy,” (Chatal, CRC Press 1989).

Alternatively, a fusion protein comprising the antibody and the cytotoxic agent may be made by recombinant techniques or peptide synthesis. The length of DNA may comprise respective regions encoding the two portions of the conjugate either adjacent to one another or separated by a region encoding a linker peptide which does not destroy the desired properties of the conjugate.

In another embodiment, the antibody may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

### 12) Other Amino Acid Sequence Modifications

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table A below under the heading of “preferred substitutions”. If such substitutions result in a change in biological activity, then more substantial changes, denominated “exemplary substitutions” in Table A, or as further described below in reference to amino acid classes, may be introduced and the products screened.

TABLE A
Amino Acid Substitutions

| | Exemplary | Preferred |
|---|---|---|
| Original Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | Ala | ala |
| Ser (S) | Thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and IgE. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IgE antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-IgE antibody.

### 13) Other Antibody Modifications

The antibodies of the present invention can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

C. Recombinant Preparation of Anti-IgE Antibodies

The invention also provides an isolated nucleic acid encoding apoptotic anti-IgE antibodies, vectors and host cells comprising such nucleic acid, and recombinant techniques for the production of the antibody.

For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide proves that are capable of binding specifically to genes encoding the heavy and light chains of the antibody). Many vectors are available. The vector components generally include, but are not limited to, one or more of the following, a signal sequence, an origin of replication, one or more marker genes, and enhancer element, a promoter, and a transcription termination sequence.

### (1) Signal Sequence Component

The anti-IgE antibodies of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native mammalian signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, factor leader (including Saccharomyces and Kluyveromyces-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the IgE binding antibody.

### (2) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

### (3) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the IgE binding antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding IgE binding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

### (4) Promoter Component

Expression and cloning vectors usually contain a promoter that is recognized by the host organism and is operably linked to the nucleic acid encoding the IgE binding antibody. Promoters suitable for use with prokaryotic hosts include the phoA promoter, -lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the IgE binding antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors. Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

IgE binding antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### (5) Enhancer Element Component

Transcription of a DNA encoding the IgE binding antibody of this invention by higher eukaryotes is often increased by inserting an enhancer sequence into the vector. Many enhancer sequences are now known from mammalian genes (globin, elastase, albumin, -fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the IgE binding antibody-encoding sequence, but is preferably located at a site 5′ from the promoter.

### (6) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding IgE binding antibody. One useful transcription termination component is the bovine growth hormone polyadenylation region. See WO94/11026 and the expression vector disclosed therein.

### (7) Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 April 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et al.), U.S. Pat. No. 5,789,199 (Joly et al.), and U.S. Pat. No. 5,840,523 (Simmons et al.) which describes translation initiation region (TIR) and signal sequences for optimizing expression and secretion. After expression, the antibody is isolated from the E. coli cell paste in a soluble fraction and can be purified through, e.g., a protein A or G column depending on the isotype. Final purification can be carried out similar to the process for purifying antibody expressed, e.g., in CHO cells.

In addition to prokaryotes, eukaryotic microbes such as filamentous fungi or yeast are suitable cloning or expression hosts for IgE binding antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated IgE binding antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Host cells are transformed with the above-described expression or cloning vectors for IgE binding antibody production and cultured in conventional nutrient media modified as appropriate for inducing promoters, selecting transformants, or amplifying the genes encoding the desired sequences.

### (8) Culturing the Host Cells

The host cells used to produce the IgE binding antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### (9) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available.

Mechanically stable matrices such as controlled pore glass or poly(styrene-divinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

D. Pharmaceutical Formulations

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

When the therapeutic agent is an antibody fragment, the smallest inhibitory fragment which specifically binds to the binding domain of the target protein is preferred. For example, based upon the variable region sequences of an antibody, antibody fragments or even peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90: 7889-7893 [1993]).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1 to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, supra.

Stability of the proteins and antibodies described herein may be enhanced through the use of non-toxic “water-soluble polyvalent metal salts”. Examples include Ca²⁺, Mg²⁺, Zn²⁺, Fe²⁺, Fe³⁺, Cu²⁺, Sn²⁺, Sn⁴⁺, Al²⁺ and Al³⁺. Example anions that can form water soluble salts with the above polyvalent metal cations include those formed from inorganic acids and/or organic acids. Such water-soluble salts have a solubility in water (at 20° C.) of at least about 20 mg/ml, alternatively at least about 100 mg/ml, alternatively at least about 200 mg/ml.

Suitable inorganic acids that can be used to form the “water soluble polyvalent metal salts” include hydrochloric, acetic, sulfuric, nitric, thiocyanic and phosphoric acid. Suitable organic acids that can be used include aliphatic carboxylic acid and aromatic acids. Aliphatic acids within this definition may be defined as saturated or unsaturated C₂₋₉ carboxylic acids (e.g., aliphatic mono-, di- and tri-carboxylic acids). For example, exemplary monocarboxylic acids within this definition include the saturated C₂₋₉ monocarboxylic acids acetic, proprionic, butyric, valeric, caproic, enanthic, caprylic pelargonic and capryonic, and the unsaturated C₂₋₉ monocarboxylic acids acrylic, propriolic methacrylic, crotonic and isocrotonic acids. Exemplary dicarboxylic acids include the saturated C₂₋₉ dicarboxylic acids malonic, succinic, glutaric, adipic and pimelic, while unsaturated C₂₋₉ dicarboxylic acids include maleic, fumaric, citraconic and mesaconic acids. Exemplary tricarboxylic acids include the saturated C₂₋₉ tricarboxylic acids tricarballylic and 1,2,3-butanetricarboxylic acid. Additionally, the carboxylic acids of this definition may also contain one or two hydroxyl groups to form hydroxy carboxylic acids. Exemplary hydroxy carboxylic acids include glycolic, lactic, glyceric, tartronic, malic, tartaric and citric acid. Aromatic acids within this definition include benzoic and salicylic acid.

Commonly employed water soluble polyvalent metal salts which may be used to help stabilize the encapsulated polypeptides of this invention include, for example: (1) the inorganic acid metal salts of halides (e.g., zinc chloride, calcium chloride), sulfates, nitrates, phosphates and thiocyanates; (2) the aliphatic carboxylic acid metal salts (e.g., calcium acetate, zinc acetate, calcium proprionate, zinc glycolate, calcium lactate, zinc lactate and zinc tartrate); and (3) the aromatic carboxylic acid metal salts of benzoates (e.g., zinc benzoate) and salicylates.

In some embodiments, the anti-IgE antibody is in a formulation comprising 100 mg/mL antibody, 30 mM histidine/histidine hydrochloride, 140 mM arginine hydrochloride, 0.04% (w/v) polysorbate 20, pH 5.5.

E. Methods of Treatment

Provided herein are methods for treating or preventing an IgE-mediated disorder comprising administering to a human patient an effective amount an anti-IgE antibody that binds the M1′ segment of an IgE (such as a human IgE). In some embodiments, the human patient has been diagnosed with the IgE-medicated disorder or is at risk of developing the IgE-medicated disorder. Provided herein are methods of reducing serum total IgE and/or allergen-specific IgE relative to baseline in a human comprising administering to a human patient an effective amount of an anti-IgE antibody that binds the M1′ segment of an IgE (such as a human IgE). Serum total IgE refers to a total amount of IgE present in a serum sample. Serum total IgE includes all the allergen-specific IgEs, and includes free or unbound, as well as IgE that is complexed with a binding partner (e.g., anti-IgE antibody, IgE-bearing B cells).

Provided herein are methods of preventing or reducing an allergen-induced increase in total serum IgE and/or allergen-specific IgE comprising administering to a human patient an effective amount of an anti-IgE antibody that binds the M1′ segment of an IgE (such as a human IgE). In some embodiments, the prevention or reduction of the allergen-induced increase is measured by comparing to the allergen-induced increase after treatment of the antibody to the allergen-induced increase before the treatment of the antibody. In some embodiments, the prevention or reduction of the allergen-induced increase is measured by comparing to the allergen-induced increase after treatment of the antibody to the allergen-induced increase in another patient or average increase in human patients without the antibody treatment. In some embodiments, the production of new IgE is prevented.

Provided herein are methods of preventing production of new IgE comprising administering to a human patient an effective amount of an anti-IgE antibody that binds the M1′ segment of an IgE (such as a human IgE).

In some embodiments of the methods described herein, an interval between administrations of the antibody is about one month or longer. In some embodiments, the interval between administrations is about two months, about three months, about four months, about five months, about six months or longer. As used herein, an interval between administrations refers to the time period between one administration of the antibody and the next administration of the antibody. As used herein, an interval of about one month includes four weeks. Accordingly, in some embodiments, the interval between administrations is about four weeks, about eight weeks, about twelve weeks, about sixteen weeks, about twenty weeks, about twenty four weeks, or longer. In some embodiments, the treatment includes multiple administrations of the antibody, wherein the interval between administrations may vary. For example, the interval between the first administration and the second administration is about one month, and the intervals between the subsequent administrations are about three months. In some embodiments, the interval between the first administration and the second administration is about one month, the interval between the second administration and the third administration is about two months, and the intervals between the subsequent administrations are about three months.

In some embodiments, the anti-IgE antibody described herein is administered at a flat dose. In some embodiment, the anti-IgE antibody described herein is administered to a human patient at a dosage from about 150 to about 450 mg per dose. In some embodiment, the anti-IgE antibody is administered to a human patient at a dosage of about any of 150 mg, 200 mg, 250 mg, 300 mg, 350 mg, 400 mg, and 450 mg per dose. Any of the dosing frequency described above may be used.

In some embodiments, the administration of the antibody to the human patient reduces the serum total IgE and/or allergen-specific IgE in the human patient. In some embodiments, the serum total IgE is reduced by at least about 20% from the baseline. In some embodiments, the serum total IgE is reduced by at least about 25% from the baseline. In some embodiments, the reduction of serum total IgE is sustained for at least one month, at least two months, at least three months, at least four months, at least five month, at least six months, or longer after the last administration of the antibody. In some embodiments, the allergen-specific IgE is reduced from baseline. In some embodiments, the administration of the antibody to the human patient prevents or reduces an allergen-induced increase in serum total IgE and/or allergen-specific IgE. In some embodiments, the prevention or reduction of allergen-induced increase in serum total IgE and/or allergen-specific IgE is sustained for at least one month, at least two months, at least three months, at least four months, at least five months, at least six months or longer after the last administration of the antibody. Methods known in the art and described herein may be used for measuring serum total IgE and allergen-specific IgE levels.

In some embodiments, the administration of the antibody has at least one of the following effects: 1) reduces exacerbation rate by ≧50% as compared to placebo, and at least one of the following: improves FEV1 by ≧5%, reduces symptom frequency or severity compared to placebo within 12 weeks of first dose and after 36 weeks of active dosing, and increases well controlled weeks compared to placebo over 24-36 weeks of active dosing; 2) reduces exacerbation rate by ≧50% as compared to placebo, improves FEV1 by <5%, no reduction in symptom frequency or severity compared to placebo within 12 weeks of first dose and after 36 weeks of active dosing, and no change in well controlled weeks compared to placebo over 24-36 weeks of active dosing; 3) reduces exacerbation rate by 40-49% as compared to placebo, improves FEV1 by <5%, and at least one of the following: reduces symptom frequency or severity compared to placebo within 12 weeks of first dose and after 36 weeks of active dosing, and increases well controlled weeks compared to placebo over 24-36 weeks of active dosing; 4) reduces exacerbation rate by 40-49% as compared to placebo, and at least one of the following: improves FEV1 by ≧5%, reduces symptom frequency or severity compared to placebo within 12 weeks of first dose and after 36 weeks of active dosing, and increases well controlled weeks compared to placebo over 24-36 weeks of active dosing; and 5) reduces exacerbation rate by <40% as compared to placebo, and at least two of the following: improves FEV1 by ≧5%, reduces symptom frequency or severity compared to placebo within 12 weeks of first dose and after 36 weeks of active dosing, and increases well controlled weeks compared to placebo over 24-36 weeks of active dosing.

As used herein, a baseline level (such as baseline level for serum total IgE, and allergen-specific IgE) in a human refers to the level before an administration of an anti-IgE antibody described herein to the human.

For the prevention or treatment of disease, the appropriate dosage of an active agent, will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

A preferred method of treatment is the treatment of IgE-mediated disorders. IgE mediated disorders includes atopic disorders, which are characterized by an inherited propensity to respond immunologically to many common naturally occurring inhaled and ingested antigens and the continual production of IgE antibodies. Specific atopic disorders include allergic asthma, allergic rhinitis, atopic dermatitis and allergic gastroenteropathy. Atopic patients often have multiple allergies, meaning that they have IgE antibodies to, and symptoms from, many environmental allergens, including pollens, fungi (e.g., molds), animal and insect debris and certain foods. However disorders associated with elevated IgE levels are not limited to those with an inherited (atopic) etiology. Other disorders associated with elevated IgE levels, that appear to be IgE-mediated and are treatable with the formulations of this present invention include hypersensitivity (e.g., anaphylactic hypersensitivity), eczema, urticaria, allergic bronchopulmonary aspergillosis, parasitic diseases, hyper-IgE syndrome, ataxia-telangiectasia, Wiskott-Aldrich syndrome, thymic alymphoplasia, IgE myeloma and graft-versus-host reaction.

Allergic rhinitis, also known as allergic rhinoconjunctivitis or hay fever, is the most common manifestation of an atopic reaction to inhaled allergens, the severity and duration of which is often correlative with the intensity and length of exposure to the allergen. It is a chronic disease, which may first appear at any age, but the onset is usually during childhood or adolescence. A typical attack consists of profuse watery rhinorrhea, paroxysmal sneezing, nasal obstruction and itching of the nose and palate. Postnasal mucus drainage also causes sore throat, throat clearing and cough. There can also be symptoms of allergic blepharoconjunctivitis, with intense itching of the conjunctivae and eyelids, redness, tearing, and photophobia. Severe attacks are often accompanied by systemic malaise, weakness, fatigue, and sometimes, muscle soreness after intense periods of sneezing.

Asthma, also known as reversible obstructive airway disease, is characterized by hyperresponsiveness of the tracheobronchial tree to respiratory irritants and bronchoconstrictor chemicals, producing attacks of wheezing, dyspnea, chest tightness, and cough that are reversible spontaneously or with treatment. It is a chronic disease involving the entire airway, but varies in severity from occasional mild transient episodes to severe, chronic, life-threatening bronchial obstruction. Asthma and atopy may coexist, but only about half of asthmatics are also atopic, and an even smaller percentage of atopic patients also have asthma. However, atopy and asthma are not entirely independent in that asthma occurs more frequently among atopic than amongst nonatopic individuals, especially during childhood. Asthma has further been historically broken down into two subgroups, extrinsic asthma and intrinsic asthma.

Extrinsic asthma, also known as allergic, atopic or immunologic asthma, is descriptive of patients that generally develop asthma early in life, usually during infancy or childhood. Other manifestations of atopy, including eczema or allergic rhinitis often coexist. Asthmatic attacks can occur during pollen seasons, in the presence of animals, or on exposure to house dust, feather pillows, or other allergens. Skin tests show positive wheal-and-flare reactions to the causative allergens. Interestingly, serum total IgE concentrations are frequently elevated, but are sometimes normal.

Intrinsic asthma, also known as nonallergic or idopathic asthma, typically first occurs during adult life, after an apparent respiratory infection. Symptoms include chronic or recurrent bronchial obstruction unrelated to pollen seasons or exposure to other allergens. Skin tests are negative to the usual atopic allergens, serum IgE concentration is normal. Additional symptoms include sputum blood and eosinophilia. Other schemes for classifying asthma into subgroups, like aspirin-sensitive, exercise-induced, infectious and psychologic merely define external triggering factors that affect certain patients more so than others.

Finally, it is important to note that while some classifications have historically associated only allergic asthma with IgE dependency, there is now strong statistically significant data showing a correlation between IgE and asthma (both allergic and non-allergic). Chapter 27, “The Atopic Diseases”, A.I. Terr in Medical Immunology, 9th Ed., Simon and Schuster, Stites et al, Ed. (1997). As a result, the term “IgE-mediated disorders”, for purposes of this patent application, includes both allergic and non-allergic asthma.

Physical signs of an asthma attack include tachypnea, audible wheezing, and use of the accessory muscles of respiration. Rapid pulse and elevated blood pressure are also typically present, as are elevated levels of eosinophils in the peripheral blood and nasal secretions. Pulmonary functions show a decrease in flow rates and 1 second forced expiratory volume (FEV₁). The total lung capacity and functional residual capacity are typically normal or slightly increased, but may be decreased with extreme bronchospasm.

The pathology of asthma can be distinguished by early phase and late phase reactions. The early phase is characterized by smooth muscle contraction, edema and hypersecretion, while the late phase reactions are characterized by cellular inflammation. Asthma can be induced by various non-specific triggers including infections (e.g., viral respiratory infections), physiologic factors (e.g., exercise, hyperventilation, deep breathing, psychologic factors), atmospheric factors (e.g., sulfur dioxide, ammonia, cold air, ozone, distilled water vapor), ingestants (e.g., propranolol, aspirin, nonsteroidal anti-inflammatory drugs), experimental inhalants (e.g., hypertonic solutions, citric acid, histamine, methacholine, prostaglandin F_{2α}) and occupational inhalants (e.g., isocyanates). Various additional occupational or environmental allergens that cause allergic asthma can include animal products, insect dusts, sea creatures, plant products, fruits, seeds, leaves and pollens, organic dyes and inks, microbial agents, enzymes, therapeutic agents, sterilizing agents, and inorganic and organic chemicals.

Atopic dermatitis, also known as eczema, neurodermatitis, atopic eczema or Besnier's prurigo, is common chronic skin disorder specific to a subset of patients with the familial and immunologic features of atopy. The essential feature is a pruritic dermal inflammatory response, which induces a characteristic symmetrically distributed skin eruption with predilection for certain sites. There is also frequent overproduction of IgE by plasma cells. While atopic dermatitis is classified as a cutaneous form of atopy because it is associated with allergic rhinitis and asthma and high IgE levels, the severity of the dermatitis, however, does not always correlate with exposure to allergens on skin testing, and desensitization (unlike other allergic diseases) is not effective treatment. While high serum IgE is confirmatory of a diagnosis of allergic asthma, normal levels do not preclude it. Onset of the disease can occur at any age, and lesions begin acutely with erythematous edematous papule or plaque with scaling. Itching leads to weeping and crusting, then to chronic lichenification. On the cellular level, acute lesion is edemous and the dermis is infiltrated with mononuclear cells, CD4 lymphocytes. Neutrophils, eosinophils, plasma cells and basophils are rare, but degranulated mast cells are present. Chronic lesions feature epidermal hyperplasia, hyperkeratosis and parakeratosis, and the dermis is infiltrated with mononuclear cells, Langerhans' cells and mast cells. There may also be focal areas of fibrosis, including involvement of the perineurium of small nerves.

Allergic gastroenteropathy, also known as eosinophilic gastroenteropathy, is an unusual atopic manifestation in which multiple IgE food sensitivities are associated with a local gastrointestinal tract mucosal reaction. It is rare in adults, but more common, but transient, in infants. The condition results when ingested food allergens react with local IgE antibodies in the jejunal mucosa liberate mast cell mediators, resulting in gastrointestinal symptoms shortly after the meal. Continued exposure produced chronic inflammation, resulting in gastrointestinal protein loss and hypoproteinemic edema. Blood loss through the inflamed intestinal mucosa may be significant enough to cause iron deficiency anemia. The allergic reaction occurs locally in the upper gastrointestinal mucosa following allergen exposure, but resolves with allergen avoidance.

Anaphylaxis and urticaria are clearly IgE-mediated, but they lack genetic determinants, and have no predilection for atopic individuals. Anaphylaxis is an acute, generalized allergic reaction with simultaneous involvement of several organ systems, usually cardiovascular, respiratory, cutaneous and gastrointestinal. The reaction is immunologically mediated, and it occurs on exposure to an allergen to which the subject has been previously sensitized. Urticaria and angioedema refers to the physical swelling, erythema and itching resulting from histamine stimulated receptor in superficial cutaneous blood vessels, and is the hallmark cutaneous feature of systemic anaphylaxis. Systemic anaphylaxis is the occurrence of an IgE-mediated reaction simultaneously in multiple organs resulting from drug, insect venom or food. It is caused suddenly by allergen induced, mast cell loaded IgE, resulting in profound and life-threatening alteration in the functioning of various vital organs. Vascular collapse, acute airway obstruction, cutaneous vasodilation and edema, and gastrointestinal and genitourinary muscle spasm occur almost simultaneously, although not always to the same degree.

The pathology of anaphylaxis includes angioedema and hyperinflated lungs, with mucous plugging of airways and focal atelectasis. On a cellular level, the lungs appear similarly as during an acute asthma attack, with hypersecretion of bronchial submucosal glands, mucosal and submucosal edema, peribronchial vascular congestion and eosinophilia in the bronchial walls. Pulmonary edema and hemorrhage may be present. Bronchial muscle spasm, hyperinflation, and even rupture of alveoli may also be present. Important features of human anaphylaxis include edema, vascular congestion, and eosinophilia in the lamina propria of the larynx, trachea, epiglottis and hypopharynx.

Exposure to the allergen may be through ingestion, injection, inhalation or contact with skin or mucous membrane. The reaction begins within seconds or minutes after exposure to the allergen. There may be an initial fright or sense of impending doom, followed rapidly by symptoms in one or more target organ systems: cardiovascular, respiratory, cutaneous or gastrointestinal.

The allergens responsible for anaphylaxis differ from those commonly associated with atopy. Foods, drugs, insect venoms or latex are the common sources. Food allergens includes those found in crustaceans, mollusks (e.g., lobster, shrimp, crab), fish, legumes (e.g., peanuts, peas, beans, licorice), seeds (e.g. sesame, cottonseed, caraway, mustard, flaxseed, sunflower), nuts, berries, egg whites, buckwheat and milk. Drug allergens include those found in heterologous proteins and polypeptides, polysaccharides and haptenic drugs. Insect allergens include Hymenoptera insects, including the honeybee, yellow jacket, hornet, wasp and fire ant.

While epinephrine is the typical treatment for anaphylaxis, antihistamine or other histamine blockers are typically prescribed for less severe urticaria or angioedemic reaction.

F. Combination Therapies

The method of the invention can be combined with known methods of treatment for IgE-mediated disorder, either as combined or additional treatment steps or as additional components of a therapeutic formulation.

For example, antihistamines, especially non-sedating antihistamines may be administered before, prior to, or commensurate with the anti-IgE antibodies of the invention. Suitable antihistamines include those of the alkylamine (e.g., chlorpheniramine), ethanolamine (e.g., diphenhydramine) and phenothiazine (e.g., promethazine). While many antihistamines antagonize the pharmacological effects of histamine by blocking its receptor sites on the effector cells, other common antihistamine drugs operate by blocking histamine release from mast cells that have been sensitized and armed with allergen-specific IgE (e.g., cromolyn sodium). Example antihistamines include astemizole, azatadine maleate, bropheniramine maleate, carbinoxamine maleate, cetirizine hydrochloride, clemastine fumarate, cyproheptadine hydrochloride, dexbrompheniramine maleate, dexchlorpheniramine maleate, dimenhydrinate, diphenhydramine hydrochloride, doxylamine succinate, fexofendadine hydrochloride, terphenadine hydrochloride, hydroxyzine hydrochloride, loratidine, meclizine hydrochloride, tripelannamine citrate, tripelennamine hydrochloride, triprolidine hydrochloride.

Particular symptoms of IgE-mediated disorders (e.g., early phase reactions) can be ameliorated with sympathomimetics or drugs having bronchodialator effect. Epinephrine is a broad acting alpha and beta-adrenergic often administered subcutaneously in a dose of 0.2-0.5 mL of 1:100 aqueous solution. A longer acting form of epinephrine (i.e., terbutaline) in 1:200 suspension is also used when a longer duration effect is desired. Suitable additional beta-adrenergics include albuterol, pirbuterol, metaproterenol, salmeterol, isoetharine and formeterol for administration nasally (e.g., hand-held nebulizer, intermittent positive-pressure breathing device, or metered-dose pressurized inhalers) or orally.

Bronchodilation can also be achieved through administration of xanthines, especially when they are administered in combination with the above sympathomimetic drugs. Example xanthines include aminophylline (iv. 250-500 mg) and theophylline (oral, 10-20 μg/ml serum concentration).

Other symptoms from various IgE-mediated disorders (e.g., late phase reactions) can be attenuated by treatment with glucocorticoids or other drugs having anti-inflammatory effects. Prednisone (30-60 mg daily) is administered systemically for severe attacks, while beclomethasone dipropionate, triamcinolone acetonide and flunisolide are administered in aerosolized form as long-term maintenance therapy. Additional corticosteroids that have anti-inflammatory effects include: betamethasone, budesonide, dexamethasone, fludrocortisone acetate, flunisolide, fluticasone propionate, hydrocortisone, methylprednisolone, prednisolone, prednisone, triamcinolone.

Non-steroidal anti-inflammatory drugs that may also be used in combination with the therapeutic methods of the invention include, acetaminophen, aspirin, bromfenac sodium, diclofenac sodium, diflunisal, etodolac, fenoprofen calcium, flurbiprofen, ibuprofen, indomethacin, ketoprofen, meclofenamate sodium, mefenamic acid, nabumetone, naproxen, naproxen sodium, oxyphenbutazone, phenylbutzone, piroxicam, sulindac, tolmetin sodium.

Additionally, the maximum therapeutic benefit may also be achieved with the administration of decongestants (e.g., phenylephrine, phenylpropanolamine, pseudoephadrin), cough suppressants (e.g., dextromethorphan, codeine, or hydrocodone) or analgesic (e.g., acetaminophen, aspirin). Allergen desensitization is a treatment form in which allergens are injected into the patient for the purpose or reducing or eliminating the allergic response. It is also known as allergen immunotherapy, hyposensitization or allergy injection therapy. It is often used in combination with other allergy treatments, but not often as a primary treatment. It has been successfully employed when allergen avoidance is impossible. A typical allergen desensitization treatment incorporates subcutaneous injection of sterile allergen in increasing doses once or twice a week until a dose is achieved that produces a transient small local area of inflammation at the injection site. The dose is then given on a maintenance schedule once every 2-4 weeks. Allergic desensitization is most often used in the treatment of allergic asthma and allergic rhinitis, although it has had success in treating anaphylaxis. Desensitization has also been effectively used through the use of adjuvants, such as incomplete Freund's adjuvant, which is an emulsion of aqueous antigen in mineral oil. The physiological effect creates an insoluble liquid depot from which droplets of allergen are gradually released. Another form of allergen desensitization is to polymerize monomeric allergens with glutaraldehyde to create a molecule with relatively low allergenicity (i.e., causes allergic response), while retaining an effective degree of immunogenicity.

G. Pharmaceutical Dosages and Administration

Dosages and desired drug concentration of pharmaceutical compositions of the present invention may vary depending on the particular use envisioned. In some embodiments, the dosage for the anti-IgE antibody is from about 150 mg to about 450 mg per dose for a human patient. In some embodiments, the dosage is about any of 150 mg, 200 mg, 250 mg, 300 mg, 350 mg, 400 mg, or 450 mg per dose for a human patient. In some embodiments, the antibody is administered subcutaneously or intravenously. In some embodiments, the antibody is administered at monthly intervals or greater than monthly intervals. In some embodiments, the antibody is administered at quarterly intervals. The progress of the therapy may be monitored during the treatment by conventional techniques and assays, such as measuring serum total IgE levels, allergen-specific IgE levels, allergen-induced increase in serum total IgE and allergen-specific IgE, etc.

In some embodiments, the anti-IgE antibodies described herein are administered to the mammal by subcutaneous (i.e. beneath the skin) administration. For such purposes, the formulation may be injected using a syringe. However, other devices for administration of the formulation are available such as injection devices (e.g. the INJECT-EASE™ and GENJECT™ devices); injector pens (such as the GENPEN™); auto-injector devices, needleless devices (e.g. MEDIJECTOR™ and BIOJECTOR™); and subcutaneous patch delivery systems.

H. Articles of Manufacture and Kits

In another aspect, an article of manufacture or kit is provided which comprises an anti-IgE antibody described herein. The article of manufacture or kit may further comprise instructions for use of the antibody in the methods of the invention. Thus, in certain embodiments, the article of manufacture or kit comprises instructions for the use of an anti-IgE antibody in methods for treating or preventing an IgE-mediated disorder in an individual comprising administering to the individual an effective amount of an anti-IgE antibody. In certain embodiments, the individual is a human. In some embodiments, the individual has severe, moderate, or mild asthma.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic.

The container holds the formulation. The article of manufacture or kit may further comprise a label or a package insert, which is on or associated with the container, may indicate directions for reconstitution and/or use of the formulation. The label or package insert may further indicate that the formulation is useful or intended for subcutaneous, intravenous, or other modes of administration for treating or preventing an IgE-mediated disorder in an individual. The container holding the formulation may be a single-use vial or a multi-use vial, which allows for repeat administrations (e.g. from 2-6 administrations) of the reconstituted formulation. The article of manufacture or kit may further comprise a second container comprising a suitable diluent (e.g., BWFI). Upon mixing the diluent and the lyophilized formulation, the final protein, polypeptide, or small molecule concentration in the reconstituted formulation will generally be at least 50 mg/ml. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic protein or antibody, including both single or multi-chambered pre-filled syringes. Exemplary pre-filled syringes are available from Vetter GmbH, Ravensburg, Germany.

The article of manufacture or kit herein optionally further comprises a container comprising a second medicament, wherein the anti-IgE antibody is a first medicament, and which article or kit further comprises instructions on the label or package insert for treating the subject with the second medicament, in an effective amount. The second medicament may be any of those set forth above, with an exemplary second medicament being an anti-IgE antibody, an antihistamine, a bronchodilator, a glucocorticoid, an NSAID, a decongestant, a cough suppressant, an analgesic, a TNF-antagonist, an integrin antagonist, an immunosuppressive agent, an IL-4 antagonist, an IL-13 antagonist, a dual IL-4/IL-13 antagonist, a DMARD, an antibody that binds to a B-cell surface marker, and a BAFF antagonist.

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

The invention will be more fully understood by reference to the following examples. They should not, however, be construed as limiting the scope of the invention. All citations throughout the disclosure are hereby expressly incorporated by reference.

### EXAMPLES

### Example 1

### Anti-M1 Prime Monoclonal Antibody, MEMP1972A, Treatment Reduced Serum IgE in Healthy Volunteers and Subjects with Allergic Rhinitis in a Phase 1a/b Study

Elevated IgE levels are associated with allergic disease including allergic asthma. Membrane IgE includes a sequence termed ‘M1 prime’, which is present in human IgE-switched B-cells, IgE memory B-cells, and IgE plasmablasts. MEMP1972A is a humanized monoclonal antibody targeting M1 prime, which depletes M1-prime expressing cells via apoptosis and/or antibody-dependent cell-mediated cytotoxicity mechanisms in vitro, therefore reducing IgE levels without affecting other immunoglobulin isotypes. Development of MEMP1972A for the treatment of allergic asthma was initiated with Phase 1 trials to test safety in healthy volunteers and allergic subjects.

Methods

Two Phase 1, randomized, blinded, placebo-controlled studies investigated the safety, tolerability, pharmacokinetics and pharmacodynamics of MEMP1972A in (1) healthy adult volunteers (n=31 MEMP1972A, n=14 placebo) and (2) adult subjects with allergic rhinitis (n=24 MEMP1972A, n=12 placebo [NCT01160861]).

### Phase 1A Study.

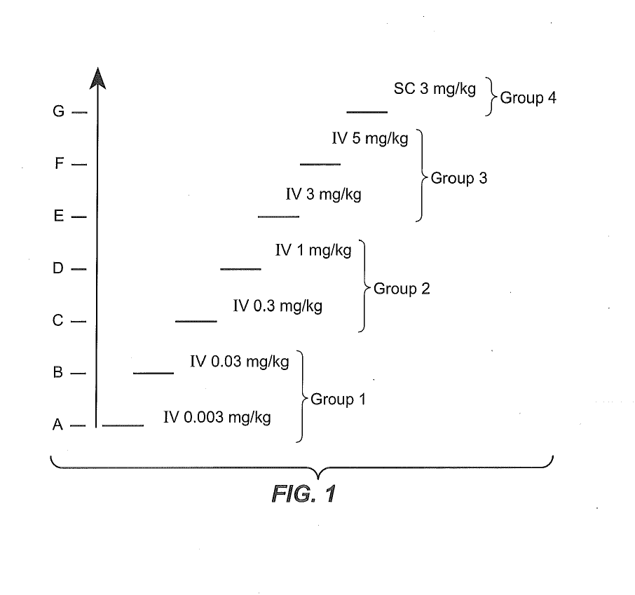

A total of 45 healthy volunteers were enrolled into seven predefined single ascending-dose cohorts of 7 subjects each (Cohorts A-G) (Table 1). Subjects were screened to assess their eligibility to enter the study within 35 days before Day 1 of treatment and eligible subjects were required to check into the clinic the day before treatment (Day −1). Each eligible subject was randomly assigned to receive a single intravenous (IV) or subcutaneous (SC) dose of MEMP1972A or matching placebo according to a single ascending dose escalation schema (FIG. 1).

TABLE 1
Study Cohorts for Phase 1A study

| | | | | Number of | Number of |
|---|---|---|---|---|---|
| | Dose | Total Doses | | Subjects | Subjects |
| Cohort | (mg/kg) | Administered | Route | (active drug) | (placebo) |
| A | 0.003 | 1 | IV | 3 | 2 |
| B | 0.03 | 1 | IV | 3 | 2 |
| C | 0.3 | 1 | IV | 5 | 2 |
| D | 1.0 | 1 | IV | 5 | 2 |
| E | 3.0 | 1 | IV | 5 | 2 |
| F | 5.0 | 1 | IV | 5 | 2 |
| G | 3.0 | 1 | SC | 5 | 2 |

MEMP1972A was produced using Chinese hamster ovary (CHO) cells, purified and formulated as 100 mg/mL MEMP1972A in 30 mM histidine/histidine hydrochloride, 140 mM arginine hydrochloride, and 0.04% (w/v) polysorbate 20 at pH 5.5 with water for injection. The MEMP1972A drug product was supplied as a sterile preservative-free liquid solution for IV and SC administration in a single-use, 2-mL clear glass vial that was stoppered with a 13-mm fluoro-resin laminated stopper and capped with an aluminum cap with a flip-off plastic seal. Each vial contained 150 mg of active pharmaceutical ingredient (API). Diluent and matching placebo for MEMP1972A contained the same excipients as the drug product, without API. Placebo was supplied in a vial configuration identical to the drug product. Diluent was supplied in a 50-mL clear glass vial that is stoppered with a 20-mm fluoro-resin laminated stopper and capped with an aluminum cap with a flip-off plastic seal containing 25 mL of diluent. MEMP1972A was administered as a single dose ranging from 0.003 to 5 mg/kg IV or 3 mg/kg SC on Day 1. MEMP1972A, placebo, and diluent vials were refrigerated at 2° C.-8° C. until use. Dose preparations for IV infusions of Cohorts A-D were done by diluting MEMP1972A or placebo with diluent into an empty sterile vial. Once diluted using diluent, MEMP1972A or placebo solutions were stored at refrigerated or room temperatures of 2° C.-25° C. for up to 8 hours prior to use. No dilution was required for IV Cohort E, F and SC Cohort G. For subjects receiving IV doses, MEMP1972A or placebo was delivered using a syringe pump with a microbore extension set for a duration of 1 hour. For subjects receiving SC doses, MEMP1972A or placebo was administered by SC injection, using syringes (BD insulin syringe). For the administration of the 3 mg/kg dose (Cohort G), up to 1.0 mL was delivered SC per injection site using a 1.0 cc syringe with a 25 or 27 gauge needle that is ⅝ of inch long. SC injections were administered in the abdomen.

After start of study on Day 1, subjects were required to return to the clinic on Day 5 for the first follow up assessment. For the seven cohorts (A-G), physical assessments and pharmacokinetic (PK) samples were obtained on Day 1, 30 minutes predose, 0-60 minutes post study drug administration and 24 hours post study drug administration. Additional assessments and PK samples were obtained on Days 5, 14, 29, 85, and 168. Blood serum samples were assessed for total serum levels of MEMP1972A by quantitative immunoassays, for the presence of anti-therapeutic antibodies (ATA) using a bridge ELISA, and measurement of total and allergen-specific IgE using a standard clinical assay, Immulite 2000 (Siemens Medical Solutions Diagnostics, Los Angeles Calif.). See Li, et al., Ann Clin Lab Sci., 34(1):67-74 (2004).

Methods known in the art may be used to detect presence of anti-therapeutic antibodies in a serum sample. For example, for detection of antibodies to MEMP1972A in human serum, serum samples were diluted 1/50 and subjected to a bridge ELISA. A 70 μL sample of the diluted serum was loaded per well in a 96-well polypropylene microplate (Corning Inc., Lowell, Mass.) together with 70 μL of Master Mix containing 2.0 μg/mL biotinylated MEMP1972A and 2.0 μg/mL digoxigenin-conjugated MEMP1972A to capture antibodies directed against MEMP1972A. The microplate was incubated overnight for 16 to 24 hours at room temperature with agitation. The samples from the polypropylene microplate were then transferred to a streptavidin-coated 96-well Reacti-Bind High Bind microplate (Pierce, Rockford, Ill.) and incubated for 2 hours at room temperature with agitation to capture bridged complexes. After washing the wells, a mouse anti-digoxin antibody conjugated with horseradish peroxidase (HRP) was added and the samples were incubated for 1 hour at room temperature. A peroxidase substrate, tetramethyl benzidine, was subsequently added for color development, and the reaction was stopped by adding 1M phosphoric acid. The plates were read at 450 nm for detection absorbance and read at 630 nm for reference absorbance using a Elx800 reader (BioTEK, Winooski, Vt.). Antibody titers were determined by a log titer data reduction program, Watson LIMS version 7.2.0.04 (Thermo Electron Corp., Louisville, Colo.). Blood RNA samples were used to measure M1 prime mRNA expression by quantitative polymerase chain reactions (qPCR). Briefly, RNA was purified from the whole blood samples collected from patients using the PAXgene Blood RNA Kit (Qiagen Inc.). After purification, 250 ng of total RNA was reverse transcribed to cDNA by using the SuperScript VILO cDNA Synthesis Kit (11754-050, Invitrogen), according to manufacturer's instructions on a BioRad C1000 Thermal Cycler (BioRad, Hercules Calif.). For qPCR, cDNA was amplified with Forward Primer (5′-CAGCGAGCGGTGTCTGT-3′) (SEQ ID NO:42), Reverse Primer (5′-GTGGCAGAGCACCCTATCC-3′) (SEQ ID NO:41) and 6 FAM-MGB Probe (5′-CCAGCCCGGGATTT-3′) (SEQ ID NO:43 on an ABI7900HT Fast Real-Time PCR machine (Qiagen Inc.) using SDS2.3 software (Qiagen Inc.).

Inclusion criteria for eligible subjects were: Age 18-55 years; body mass index (BMI) between 18 and 32 kg/m²; weight 40-120 kg; in good health as determined by no clinically significant findings from medical history, 12-lead ECG, and vital signs including oral body temperature at 35-37.5° C., systolic blood pressure at 90-140 mm Hg, and diastolic blood pressure at 50-90 mm Hg; neutrophil counts at screening and Day −1 visit >1,600 cells/μL; platelet counts at screening and Day −1 visit >140,000 cells/μL; males or females who were surgically sterilized, post-menopausal for the previous year, or were using two acceptable methods of contraception against pregnancy through at least 6 months (>5 anticipated half-lives of MEMP1972A) after the dose of study drug; non-smokers, as well as light or occasional smokers able to pass the laboratory screenings and refrain from smoking in the designated confinement period; and deemed able to comply with requirements of the study, including the follow-up period. Exclusion criteria included: active diagnosis of asthma or allergy with evidence of recent history of symptoms and/or treatment within the previous 5 years; history of anaphylaxis, and hypersensitivity or drug allergies. Additional exclusion criteria can be found with the identifier NCT01160861 at the world wide web at clinicaltrials.gov.

### Phase 1B Study.

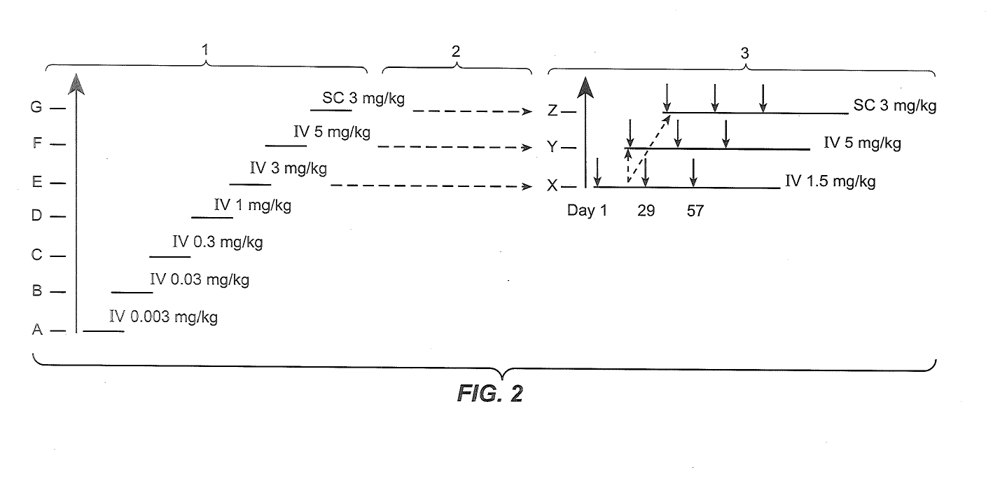

A total of 36 subjects with seasonal or perennial allergic rhinitis (SAR or PAR) were randomized based on a treatment allocation of approximately 2:1 MEMP1972A:placebo, within 3 planned multiple ascending dose cohorts (X, Y, Z) with 12 subjects per cohort (8 active and 4 placebo) (Table 2). Subjects were screened to assess their eligibility to enter the study within 35 days before Day 1 of treatment. Each eligible subject was randomly assigned to receive a single intravenous (IV) or subcutaneous (SC) dose of MEMP1972A or matching placebo according to a dose initiation and escalation schema (FIG. 2). The dose levels were 1.5 mg/kg IV in the first cohort (X), followed by 5 mg/kg IV in the second cohort (Y), and then 3 mg/kg administered SC in the third cohort (Z). Each subject received three doses administered every 4 weeks. Dosing in the first cohort (Cohort X: 1.5 mg/kg IV) was based upon review of clinical and laboratory data from the A-E cohorts in the Phase 1A study. The initiation of dosing for the second cohort (Cohort Y: 5 mg/kg IV) was based upon review of data from the F cohort in the Phase 1A study and 14 days of post-dosing follow-up data from the 1.5 mg/kg multi-dose IV Cohort (X). The initiation of dosing for the third cohort (Cohort Z: 3 mg/kg SC) was based upon review of 14 days of post-dosing follow-up data from the 3 mg/kg single-dose SC Cohort (G) in the Phase 1A study, as well as 14 days of post-first dose follow-up from all subjects in the 1.5 mg/kg multi-dose IV Cohort (X).

TABLE 2
Study Cohorts for Phase 1B study

| | | | | Number of | Number of |
|---|---|---|---|---|---|
| | Dose | Total Doses | | Subjects | Subjects |
| Cohort | (mg/kg) | Administered | Route | (active drug) | (placebo) |
| X | 1.5 | 3 | IV | 8 | 4 |
| Y | 5.0 | 3 | IV | 8 | 4 |
| Z | 3.0 | 3 | SC | 8 | 4 |

MEMP1972A was administered as a dose of 1.5 mg/kg IV, 5.0 mg/kg IV or 3.0 mg/kg SC according to the cohort study on Days 1, 29, and 57. Dose preparations for IV infusions of Cohort X (1.5 mg/kg IV) were done by diluting MEMP1972A or placebo with diluent into an empty sterile vial. No dilution was required for Cohort Y (5.0 mg/kg IV) and Cohort Z (3.0 mg/kg SC). For subjects receiving IV doses, MEMP1972A or placebo was delivered using a syringe pump with a microbore extension set for a duration of 1 hour for the first two doses (Day 1 and Day 29) and for a duration of 30 minutes for the third dose (Day 57). For subjects receiving SC doses, MEMP1972A or placebo was administered by SC injection, using syringes (BD insulin syringe). For the administration of the 3 mg/kg dose (Cohort Z), up to 1.0 mL was delivered SC per injection site using a 1.0 cc syringe with a 25 or 27 gauge needle that is ⅝ of inch long. SC injections were administered in the abdomen.

For Cohorts X and Y, physical assessments and pharmacokinetic (PK) samples were obtained 0-60 minutes after the end of the study drug infusion of Day 1, as well as 24 hours after dosing. For the second dose (Day 29), PK samples were obtained before dosing and 0-60 minutes after the end of the infusion. For the third and final dose (Day 57), samples were obtained before dosing, as well as 0-60 minutes after the end of the infusion. Additional PK samples were obtained on Days 8, 15, 85, 140, and 224. For Cohort Z, PK samples were obtained 0-60 minutes after the study drug injection on Day 1, as well as 24 hours after dosing. For the second and third dose (Days 29 and 57), PK samples were obtained pre-dose only. Additional PK samples were obtained on Days 8, 15, 36, 64, 85, 140, and 224. Blood serum samples were assessed for total serum levels of MEMP1972A by quantitative immunoassays, for the presence of anti-therapeutic antibodies (ATA) using a bridge ELISA (see assay described in phase 1A study), and measurement of total and allergen-specific IgE using a standard clinical assay, Immulite 2000 (Siemens Medical Solutions Diagnostics, Los Angeles Calif.). See Li, et al., Ann Clin Lab Sci., 34(1):67-74 (2004).

Blood RNA samples were used to measure M1 prime mRNA expression by quantitative polymerase chain reactions (qPCR). Briefly, RNA was purified from the whole blood samples collected from patients using the PAXgene Blood RNA Kit (Qiagen Inc.). After purification, 250 ng of total RNA was reverse transcribed to cDNA by using the SuperScript VILO cDNA Synthesis Kit (11754-050, Invitrogen), according to manufacturer's instructions on a BioRad C1000 Thermal Cycler (BioRad, Hercules Calif.). For qPCR, cDNA was amplified with Forward Primer (5′-CAGCGAGCGGTGTCTGT-3′) (SEQ ID NO:42), Reverse Primer (5′-GTGGCAGAGCACCCTATCC-3′) (SEQ ID NO:41) and 6 FAM-MGB Probe (5′-CCAGCCCGGGATTT-3′) (SEQ ID NO:43) on an ABI7900HT Fast Real-Time PCR machine (Qiagen Inc.) using SDS2.3 software (Qiagen Inc.).

Inclusion criteria for eligible subjects were: Age 18-55 years; diagnosis of seasonal or perennial allergic rhinitis; body mass index (BMI) between 18 and 32 kg/m²; weight 40-120 kg; total IgE serum level >10 IU/ml or ≧10 IU/ml and at least one allergen-specific IgE >0.1 kIU/L; in good health as determined by no clinically significant findings from medical history, 12-lead ECG, and vital signs including oral body temperature at 35-37.5° C., systolic blood pressure at 90-140 mm Hg, and diastolic blood pressure at 50-90 mm Hg; males or females who were surgically sterilized, post-menopausal for the previous year, or were using two acceptable methods of contraception against pregnancy through at least 6 months (>5 anticipated half-lives of MEMP1972A) after the dose of study drug; and deemed able to comply with requirements of the study, including the follow-up period. Exclusion criteria included: history of anaphylaxis, hypersensitivity or drug allergies; history of an asthma diagnosis requiring use of a daily controller medication or rescue use of a short-acting bronchodilator within the last 3 years; and forced expiratory volume in 1 second (FEV₁)<80% of predicted at screening. Additional exclusion criteria can be found with the identifier NCT01160861 at the world wide web at clinicaltrials.gov.

Results

MEMP1972A was well tolerated in both Phase 1a/b studies. Assessment of pharmacokinetic characteristics following single or multiple IV administration demonstrated dose-proportional exposures. Based on non-compartmental analyses, the mean terminal half-life was in the range of 20-21 days and the mean clearance was 2.2-2.7 mL/day/kg across the two studies.

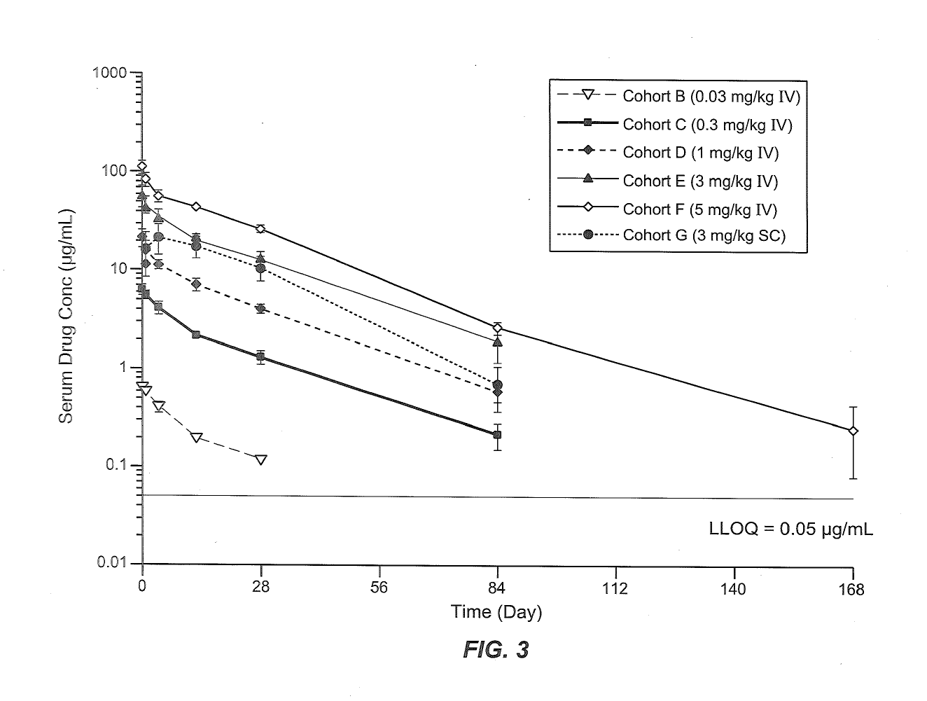

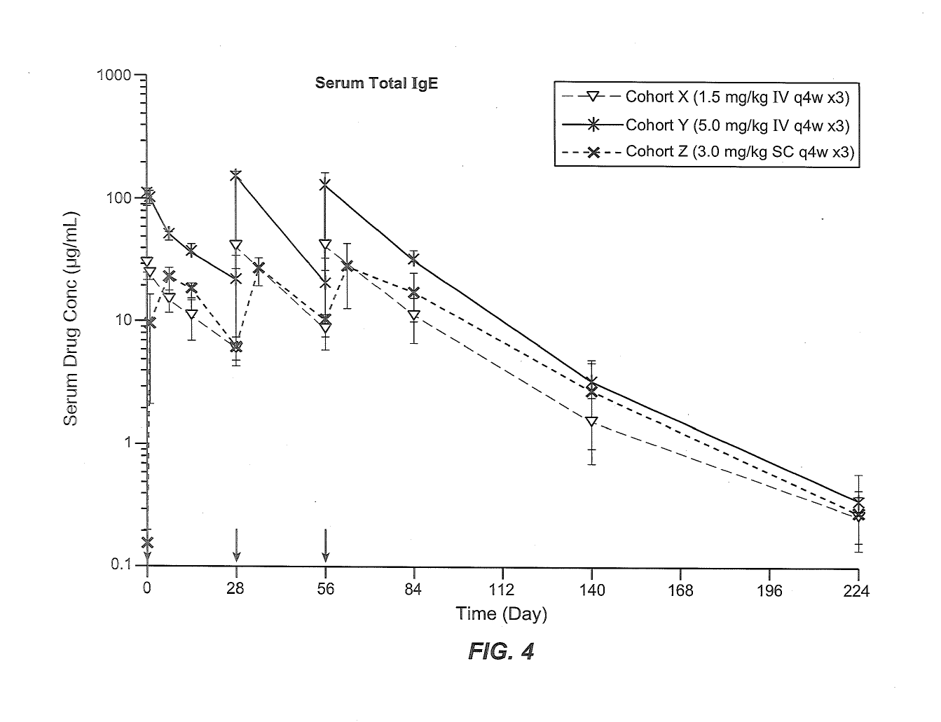

In the Phase 1a study, after IV administration, MEMP1972A had a slow mean clearance (2.31-2.74 mL/day/kg), and a long terminal half-life (˜21 days), in line with data for an IgG1 monoclonal antibody. The volume of distribution ranged from 66.6 to 81.6 ml/kg. Mean serum concentrations were dose proportional (FIG. 3). The total exposure (AUC_{0-inf}) and Cₘₐₓ showed dose proportional increases with increasing dose levels across the IV cohorts from 0.3 to 5.0 mg/kg (Table 3). The relative SC bioavailability was 66.4%, which was estimated as the ratio of the mean AUC_{0-inf} for the IV and SC cohorts of the same dose level. In the Phase 1b study, MEMP1972A pharmacokinetic concentrations were dose proportional following repeated IV administration at 1.5 or 5.0 mg/kg (Table 4; FIG. 4). The relative SC bioavailability was estimated as the ratio of the dose-normalized AUC_{0-inf} for the SC dose to those of the IV cohorts. The mean relative SC bioavailability was approximately 55.1%.

The population PK analysis of the two Phase I studies of MEMP1972A demonstrated a mean terminal half-life of 19.6 days, clearance (CL) of 216 mL/day and central volume of distribution (V_{c}) of 3.5 L following IV administration, and bioavailability of 68.8% after SC administration. Body weight was found to be a significant covariate for CL and V_{c}.

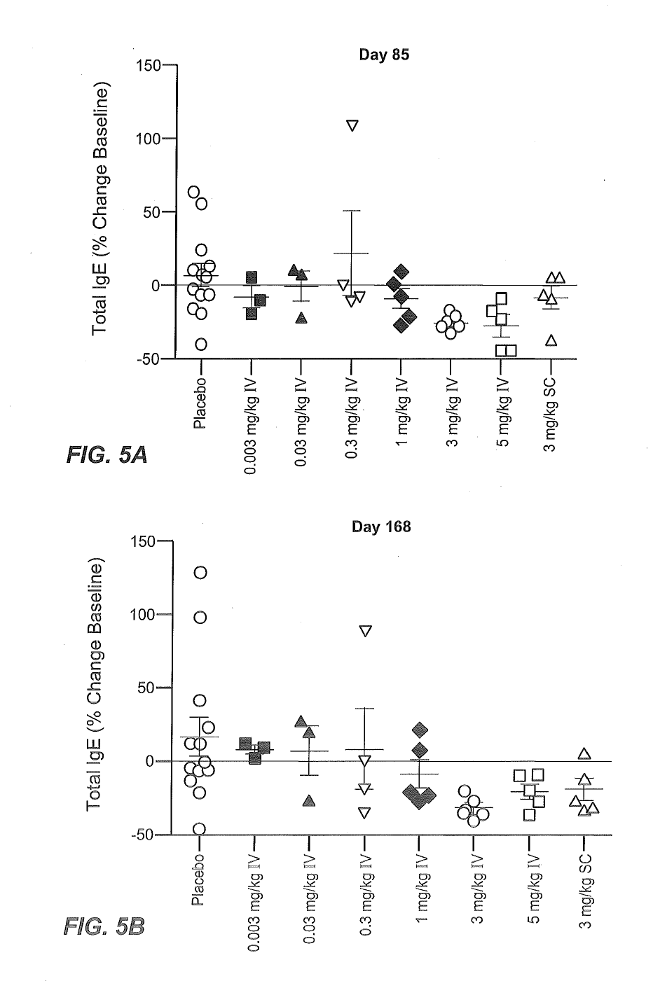

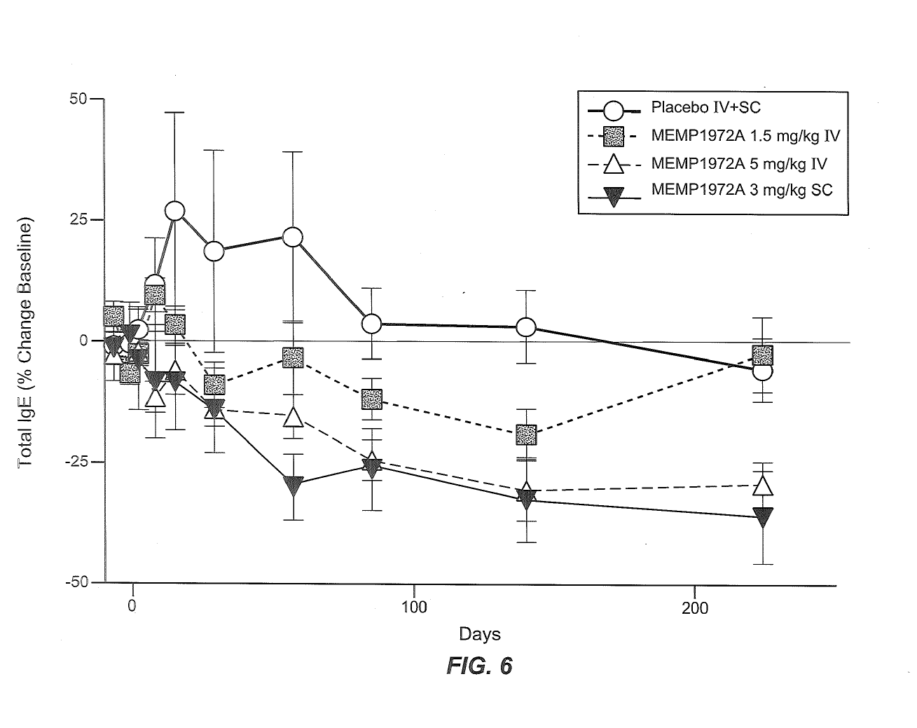

Treatment with MEMP1972A led to a dose-dependent reduction in serum total IgE. In healthy volunteers, a single dose of MEMP1972A at 3 and 5 mg/kg IV resulted in a serum total IgE reduction of 28% and 23%, respectively, at Day 85 relative to baseline, whereas no reduction was observed in the placebo, lower IV and 3 mg/kg SC cohorts (FIG. 5A). In subjects with allergic rhinitis, three monthly doses of MEMP1972A at 5 mg/kg IV and 3 mg/kg SC resulted in a mean serum total IgE reduction of 24% and 26%, respectively, at Day 85 relative to baseline, whereas no significant reduction was observed in the placebo and 1.5 mg/kg IV cohorts (FIG. 6). Serum total IgE reduction was sustained 6 months after dosing in both studies (FIG. 5B and FIG. 6). At day 224, allergen-specific IgE was significantly reduced by 40% from baseline in the 3 mg/kg SC cohort, where areas 9%, 14% and 33% reductions from baseline were observed in the placebo, 1.5 mg/kg IV and 5 mg/kg IV patients, respectively. The IgE responses of both Phase I studies, as characterized by a PK-PD model that described the dynamics of M1 prime-expressing B cells/plasmablasts, IgE-producing plasma cells and serum IgE levels, demonstrated an IC50 of 2.7 μg/mL for the effect of MEMP1972A on B cell depletion and half-life of IgE-producing plasma cells of approximately 130 days. Taken together, these data demonstrate that targeting M1 prime-expressing B cells using MEMP1972A leads to a reduction in serum total IgE in human subjects with or without allergic disease. Furthermore, treatment with MEMP1972A led to a dose-dependent reduction in serum total IgE levels, with reductions in IgE sustained for six months after the last dose. In both Phase 1 studies, adverse event (AE) profiles were similar between the MEMP1972A and placebo treatment groups and the majority of events were mild or moderate. In the Phase 1a single ascending dose study, commonly reported AEs included headache, fatigue, vessel puncture site hematoma, and nasopharyngitis. The proportion of subjects with ≧1 treatment-emergent AE (TEAE) was comparable in the placebo-treated group (11/14; 78.6%) and the MEMP1972A group (21/31; 67.7%). Most AEs were not considered to be related to the study drug. Headache was the most frequently reported study drug-related AE (n=5 [16.1%]) in the MEMP1972A-treated subjects and n=1 [8.3%] in the IV placebo-treated group). In the Phase 1b multiple ascending dose study, the most common AEs were headache, nasal congestion, back pain, dizziness and fatigue. TEAEs were experienced by 83% (20/24) of MEMP1972A-treated subjects and 75% (9/12) of placebo-treated subjects, the majority of which were mild. A single severe TEAE was reported during the study by a subject receiving the placebo. The most frequently reported TEAEs in subjects receiving MEMP1972A were upper respiratory tract infection (n=7 [29.2%] in the MEMP1972A-treated subjects, n=2 [25%] in the IV placebo-treated group) and headache (n=6 [25.0%], n=1 IV and n=1 SC [12.5%] of the respective placebo groups). No serious adverse events were reported in either study. Anti-therapeutic antibody levels remained negative throughout the two Phase I studies for all subjects who received MEMP1972A.

TABLE 3
Pharmacokinetic parameters of MEMP1972A in healthy volunteers.

| | Dose | | | AUC_{0-inf} | Cₘₐₓ | t_{1/2} | CL or CL/F | V_{z} or V_{z}/F |
|---|---|---|---|---|---|---|---|---|
| Cohort | (mg/kg) | Route | Statistic | (μg day/mL) | (μg/mL) | (day) | (mL/day/kg) | (mL/kg) |
| C | 0.3 | IV | N | 4 | 5 | 4 | 4 | 4 |
| | | | Mean | 120 | 6.21 | 21.2 | 2.55 | 77.2 |
| | | | SD | 17.4 | 0.630 | 2.33 | 0.361 | 5.92 |
| D | 1.0 | IV | N | 5 | 5 | 5 | 5 | 5 |
| | | | Mean | 367 | 21.0 | 20.7 | 2.74 | 81.6 |
| | | | SD | 34.2 | 4.29 | 2.22 | 0.249 | 10.8 |
| E | 3.0 | IV | N | 5 | 5 | 5 | 5 | 5 |
| | | | Mean | 1140 | 57.1 | 20.1 | 2.72 | 77.9 |
| | | | SD | 229 | 14.2 | 2.07 | 0.521 | 11.5 |
| F | 5.0 | IV | N | 5 | 5 | 5 | 5 | 5 |
| | | | Mean | 2170 | 110 | 20.0 | 2.31 | 66.6 |
| | | | SD | 129 | 16.5 | 2.71 | 0.141 | 8.95 |
| G | 3.0 | SC | N | 5 | 5 | 5 | 5 | 5 |
| | | | Mean | 757 | 21.5 | 15.5 | 4.18 | 91.9 |
| | | | SD | 215 | 7.27 | 2.63 | 0.986 | 21.0 |

AUC_{0-inf} = area under the concentration-time curve from time 0 to infinity;
Cₘₐₓ = maximum observed concentration;
t_{1/2} = elimination half-life;
CL = total serum clearance for IV cohorts (C-F);
CL/F = apparent total serum clearance after SC administration (cohort G);
V_{z} = volume of distribution for IV cohorts (C-F);
V_{z}/F = the apparent volume of distribution after SC administration (cohort G).
Insufficient concentration data were obtained from the cohorts A (0.003 mg/kg IV) and B (0.03) mg/kg to estimate PK parameters.

TABLE 4
Pharmacokinetic parameters of MEMP1972A in patients with allergic rhinitis.

| | Dose | | | AUC_{0-inf} | C_{max,last} | t_{1/2} | CL or CL/F | V_{z} or V_{z}/F |
|---|---|---|---|---|---|---|---|---|
| Cohort | (mg/kg) | Route | Statistic | (μg day/mL) | (μg/mL) | (day) | (mL/day/kg) | (mL/kg) |
| X | 1.5 | IV | n | 8 | 8 | 8 | 8 | 8 |
| | | | Mean | 2180 | 40.8 | 20.9 | 2.16 | 62.7 |
| | | | SD | 418 | 7.38 | 5.55 | 0.586 | 12.4 |
| Y | 5.0 | IV | n | 8 | 8 | 8 | 8 | 8 |
| | | | Mean | 7060 | 130 | 21.4 | 2.17 | 66.7 |
| | | | SD | 1090 | 35.8 | 3.09 | 0.330 | 12.3 |
| Z | 3.0 | SC | n | 7 | 7 | 7 | 7 | 7 |
| | | | Mean | 2370 | 32.2 | 20.7 | 3.97 | 117 |
| | | | SD | 520 | 12.9 | 3.39 | 0.893 | 25.3 |

AUC_{0-inf} = area under the concentration-time curve from time 0 to infinity;
C_{max,last} = maximum observed concentration post last dose on Day 57;
t_{1/2} = elimination half-life;
CL = total serum clearance for IV cohorts (X and Y);
CL/F = apparent total serum clearance after SC administration (cohort Z);
V_{z} = volume of distribution for IV cohorts (X and Y);
V_{z}/F = apparent volume of distribution after SC administration (cohort Z).

Conclusions

In both Phase I studies, MEMP1972A was well tolerated up to 5 mg/kg IV and at 3 mg/kg SC in healthy volunteers and in patients with allergic rhinitis. AE profiles were similar between the MEMP1972A and placebo treatment groups and the majority of events were mild or moderate. Treatment with MEMP1972A led to a reduction in serum total IgE in healthy volunteers and in patients with allergic rhinitis, in the 3 mg/kg SC and 5 mg/kg IV cohorts, which was sustained approximately 6 months after the last dose.

### Example 2

### Effect of an Anti-M1 Prime Monoclonal Antibody, MEMP1972A, in a Phase 2a Proof-of-Activity Allergen Challenge Study in Subjects with Mild Asthma

MEMP1972A was evaluated in a Phase 2a study (NCT01196039) to test proof-of-activity following allergen inhalation challenge (AIC).

Methods

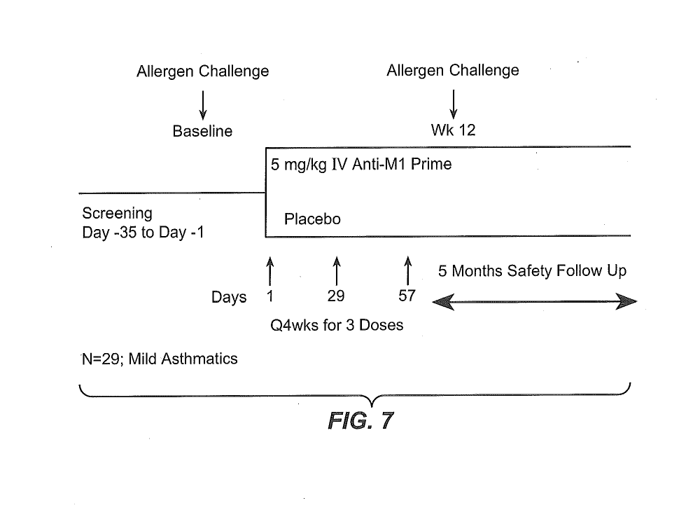

This randomized, double-blind, placebo-controlled, multicenter study evaluated the activity, safety and tolerability of MEMP1972A in subjects with mild asthma following AIC. MEMP1972A was produced using Chinese hamster ovary (CHO) cells, purified and formulated as 100 mg/mL MEMP1972A in 30 mM histidine/histidine hydrochloride, 140 mM arginine hydrochloride, and 0.04% (w/v) polysorbate 20 at pH 5.5 with water for injection. The MEMP1972A drug product was supplied as a sterile preservative-free liquid solution for IV and SC administration in a single-use, 2-mL clear glass vial that was stoppered with a 13-mm fluoro-resin laminated stopper and capped with an aluminum cap with a flip-off plastic seal. Each vial contained 150 mg of active pharmaceutical ingredient (API). Matching placebo for MEMP1972A contained the same excipients as the drug product, without API. Placebo was supplied in a vial configuration identical to the drug product. Patients with stable, mild allergic asthma were screened (Days −35 to 1) to obtain a cohort of patients with documented EAR (early asthmatic response) and LAR (late asthmatic response) to inhaled incremental challenge. Twenty-nine adult subjects were randomized (1:1) to receive placebo or MEMP1972A, which was administered intravenously at 5 mg/kg every 4 weeks for a total of 12 weeks (i.e., Days 1, 29 and 57) and followed with a five month safety follow-up (i.e., Days 57-197) (FIG. 7). Baseline and demographic characteristics were similar between the treatment groups (Table 5). Screening and blood samples were taken from subjects pre-treatment. Allergen challenge was provided pre-treatment at Day −1 and a post-treatment allergen challenge was provided on Day 86 (plus or minus 3 days), about twenty-nine days after the third and last infusion dose. For allergen challenge, ten different specific IgEs were measured in all patients (Cat-hair, cat-dander, horse, HDMf, HDMp, June Grass, Ragweed, Red Top, Sweet Vernal, Timothy) and every patient was challenged with one of those 10 allergens, depending on their skin reactivity. This relevant challenge allergen was different for each patient. Detectable allergen-specific IgE due to allergens that the subject was not challenged with were captured as irrelevant or non-challenge specific IgE levels. Irrelevant or non-challenge allergens that were highly related to the relevant challenge allergen (e.g., cat dander vs cat hair) were excluded from the irrelevant or non-challenge specific IgE evaluations. Methacholine challenges were performed 24 hours before and 24 hours following allergen challenges. Methacholine chloride stock (methacholine chloride powder for inhalation) was prepared at a concentration of 128 mg/mL in 0.9% sodium chloride (normal saline). Methacholine was further diluted in normal saline to achieve working concentrations from 0.031 mg/mL to 128 mg/mL. Methacholine inhalation was performed using the method of Cockcroft (Cockcroft et al. 1987). Briefly, subjects inhaled methacholine from a Hans Rudolf valve connected to a Wright nebulizer with an output of 0.13 mL/min. Subjects were instructed to wear nose clips and to breathe normally from the mouthpiece during the 2-minute inhalation period. Subjects inhaled normal saline, followed by doubling concentrations of methacholine for 2 minutes. FEV₁ was measured 30 and 90 seconds after each inhalation. The test ended when a 20% decline in FEV₁ of a subject's baseline value occurred and the methacholine PC₂₀ was calculated. A sputum induction procedure was performed according to the method described by Pizzichini et al. 1996 (Eur. Respir. J. 9:1174-1180) 24 hours before the allergen challenge, and 7 hours and 24 hours following each allergen challenge. Sputum samples can be measured for protein levels and mRNA expression for analysis of total IgE and M1 prime. The methacholine challenge preceded the sputum induction. Eosinophils in sputum and blood were also measured as exploratory biomarkers. Levels of eosinophils in the peripheral blood were measured using a standard complete blood count (CBC) assay. Eosinophils in the sputum were identified based on cell morphology and staining, and subsequently counted under a microscope for determination of eosinophil levels. No modification of the MEMP1972A dose levels were allowed during the study. The primary outcome measure was the area under the curve (AUC) of the allergen-induced late asthmatic response (LAR) between 3 and 7 hours after allergen challenge at week 12 (Day 86) after dose 1. Secondary outcome measures included the early asthmatic response (EAR) AUC between 0 and 3 hours or 0 and 2 hours post-treatment allergen challenge to obtain the area of the percent decline in FEV₁ over time and included the change in methacholine challenge PC₂₀ on Day 87 (24 hours after the post-dosing allergen challenge) relative to the pre-allergen challenge PC₂₀ calculated on Day 85. Serum total IgE and allergen-specific IgE were assessed to demonstrate mechanistic activity of MEMP1972A using a standard clinical assay, Phadia ImmunoCAP (Phadia Inc.). Levels of CCL17 in the serum were measured using a Human CCL17/TARC Quantikine ELISA kit (R&D Systems, Minneapolis, Minn.; catalog no. DDN100).

TABLE 5
Baseline and demographic characteristics

| | MEMP1972A | Placebo | All subjects |
|---|---|---|---|
| Baseline characteristic | (n = 15) | (n = 14) | (n = 29) |
| Mean age, years (SD) | 30.9 (10.6) | 29.4 (10.3) | 30.2 (10.3) |
| Sex, female (%) | 7 (46.7) | 10 (71.4) | 17 (58.6) |
| Race, white (%) | 13 (86.7) | 13 (92.9) | 26 (89.7) |
| Mean weight, kg (SD) | 79.13 (18.25) | 76.49 (15.90) | 77.86 (16.91) |
| Mean FEV₁, % | 90.99 (13.67) | 89.66 (12.13) | 90.35 (12.73) |
| predicted (SD) | | | |
| Mean LAR AUC, | 17.1 (6.2) | 17.8 (4.1) | 17.5 (5.2) |
| %/hr (SD) | | | |
| Mean total IgE, | 72.2 (59.7) | 89.8 (81.2) | 80.7 (70.2) |
| IU/mL (SD) | | | |

FEV₁ = forced expiratory volume in 1 second;
LAR = late asthmatic response;
AUC = area under the curve;
SD = standard deviation

Inclusion criteria for eligible subjects were: age 18 to 65 years; weight between 50 and 125 kg; mild, stable allergic asthma; history of episodic wheeze and shortness of breath; FEV₁ at baseline ≧70% of the predicted value; males or females who were surgically sterilized, post-menopausal for the previous year, or were using two acceptable methods of contraception against pregnancy through at least 5 months following the last administration of study drug; documented PC₂₀ value for prediction of the starting allergen concentration at screening; positive skin prick test to common standard aeroallergens extracts; and positive allergen-induced early and late airway response. The positive allergen-induced early airway response was defined as a ≧20% decline in FEV₁ from the highest pre-allergen value measured 0-2 hours or 0-3 hours post-allergen challenge. The late airway response was defined as a ≧15% decline in FEV₁ from the highest pre-allergen value measured 3-7 hours post-allergen challenge. Exclusion criteria included: A worsening of asthma within 6 weeks preceding Visit 1; acute respiratory infection within 6 weeks preceding Visit 2 or any ongoing chronic infection; history of recurrent bacterial infection as an adult or history or presence of any chronic infectious condition; lung disease other than mild allergic asthma; chronic use of any other medication for treatment of allergic lung disease other than short-acting β2-agonists or ipratropium bromide; use of cromoglycate, nedocromil, leukotriene receptor antagonists, and inhibitors of 5-lipoxygenase are not permitted within 4 weeks prior to Visit 2; allergen or peptide immunotherapy within 6 months prior to study treatment; and treatment with a monoclonal antibody or chimeric biomolecule within the previous 5 months, including omalizumab, at the time of Visit 2. Additional exclusion criteria can be found with the identifier NCT01196039 at the world wide web at clinicaltrials.gov.

Results

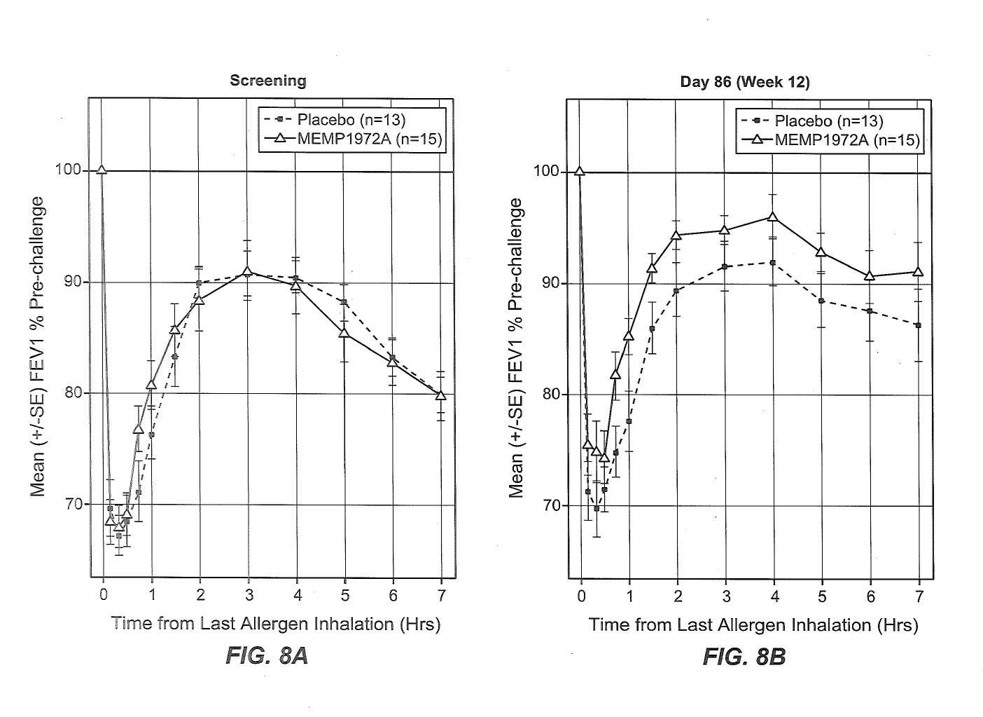

A total of 28 subjects were included in the primary endpoint analysis (n=15 MEMP1972A, n=13 placebo); one subject from the placebo group withdrew from the study due to increased asthma symptoms. At screening, the two treatment groups had similar percentage declines in forced expiratory volume in 1 second (FEV₁) assessments. At the second visit, one month after the third dose of study drug, improvements in both the EAR and LAR were observed compared with placebo. MEMP1972A treatment was well tolerated after 12 weeks in subjects with mild asthma. At Week 12, the AUC of the LAR in MEMP1972A-treated subjects was reduced by a mean of 36% vs. placebo (90% CI: -14, 69%, p=0.21) (FIGS. 8A and B). In addition, the AUC of the EAR was significantly decreased by a mean of 26% vs. placebo (90% CI: 6, 43%, p=0.046), whereas there was no effect on airway hyperresponsiveness to methacholine. The AUC of the EAR was calculated between 0 and 2 hours after allergen challenge at Week 12. The maximum LAR FEV₁ decline was 13% (90% CI: -33%, 44; p=0.58) vs. placebo (FIG. 8). The maximum EAR FEV₁ decline was 11% (90% CI: -7%, 26; p=0.27) vs. placebo (FIG. 8). Subgroup analyses demonstrated that subjects with ≧2 consecutive ≧15% drops (subgroup A) or ≧3 consecutive 10% drops in pre-challenge LAR FEV₁ (subgroup B) showed a greater response to MEMP1972A treatment, with LAR AUC reductions of 43% (90% CI: -5%, 77%) and 62% (90% CI: 29%, 88%), respectively compared with placebo (Table 6). Subjects who showed LAR AUC ≧10%/hour at screening (subgroup C) also showed an improved response, with a LAR AUC reduction of 54% (90% CI 10%, 84%) (Table 6).

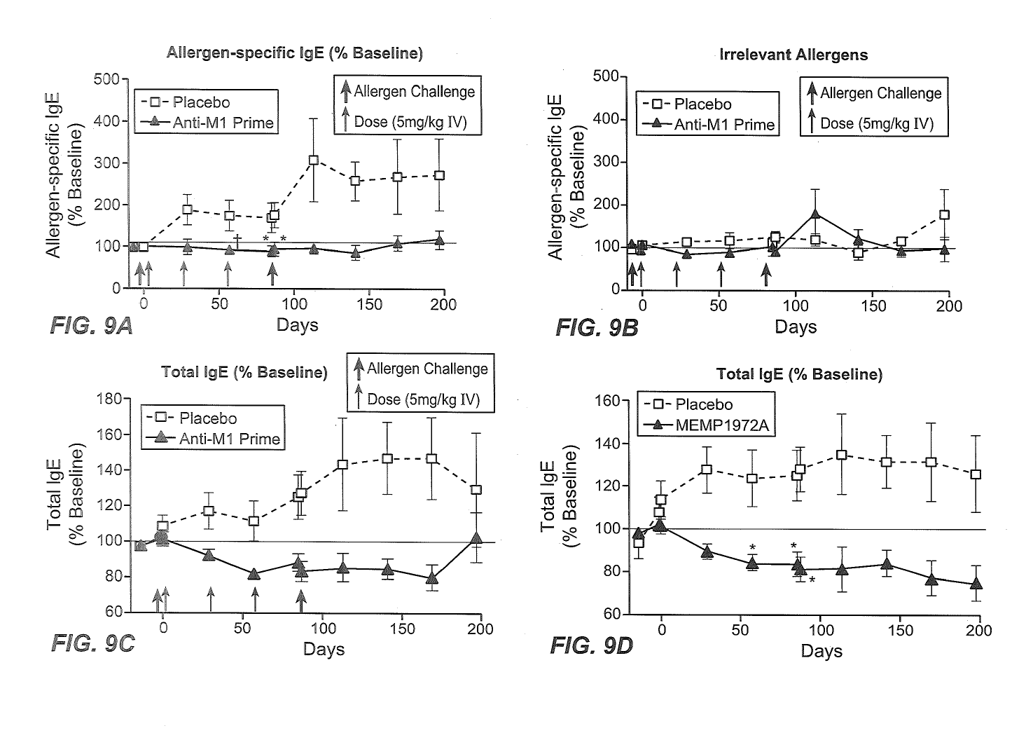

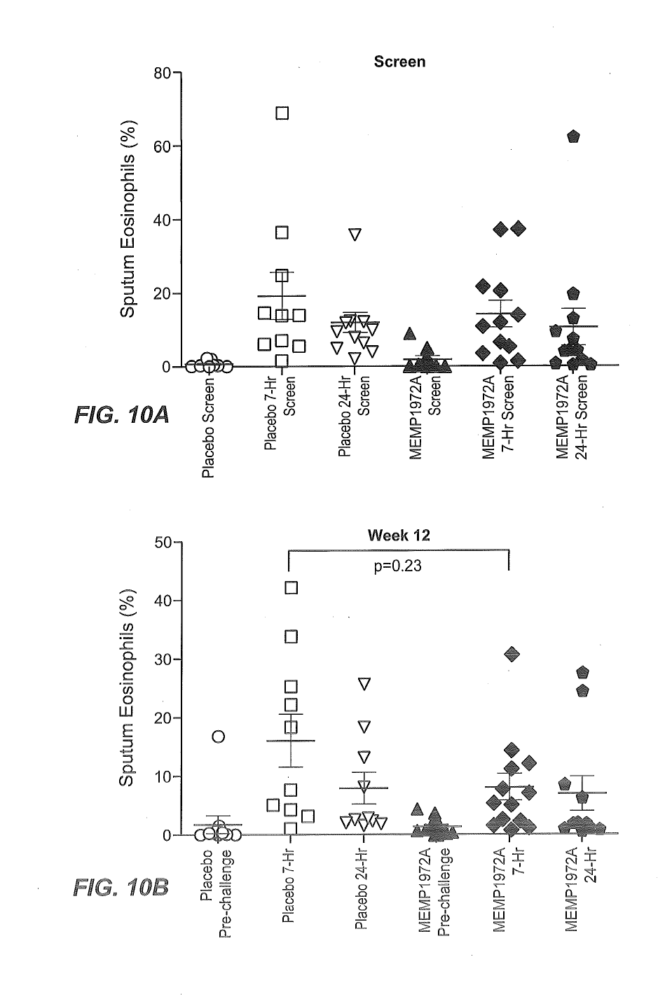

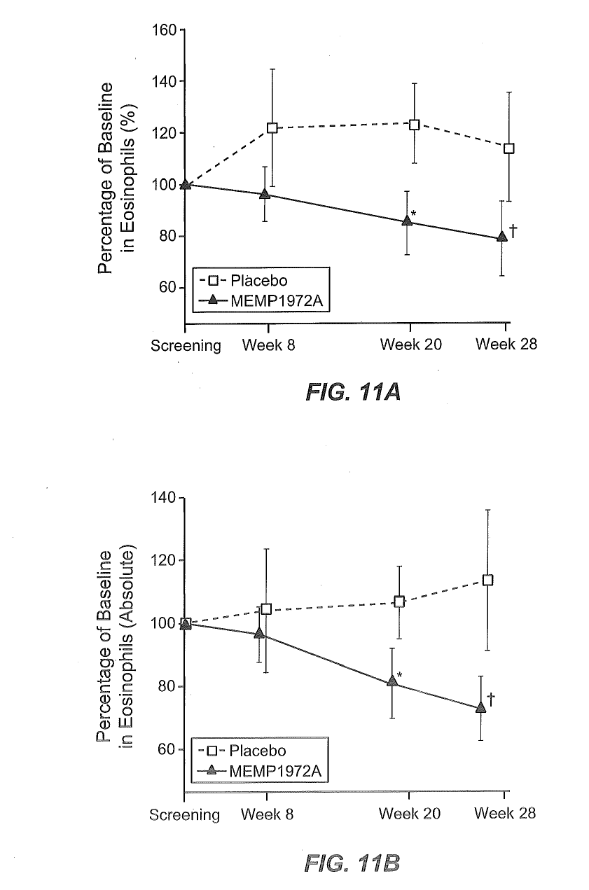

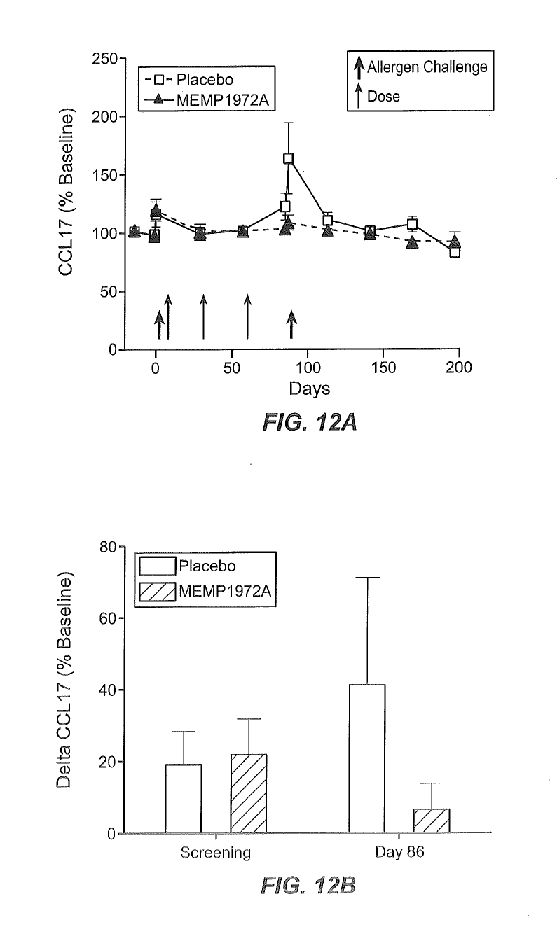

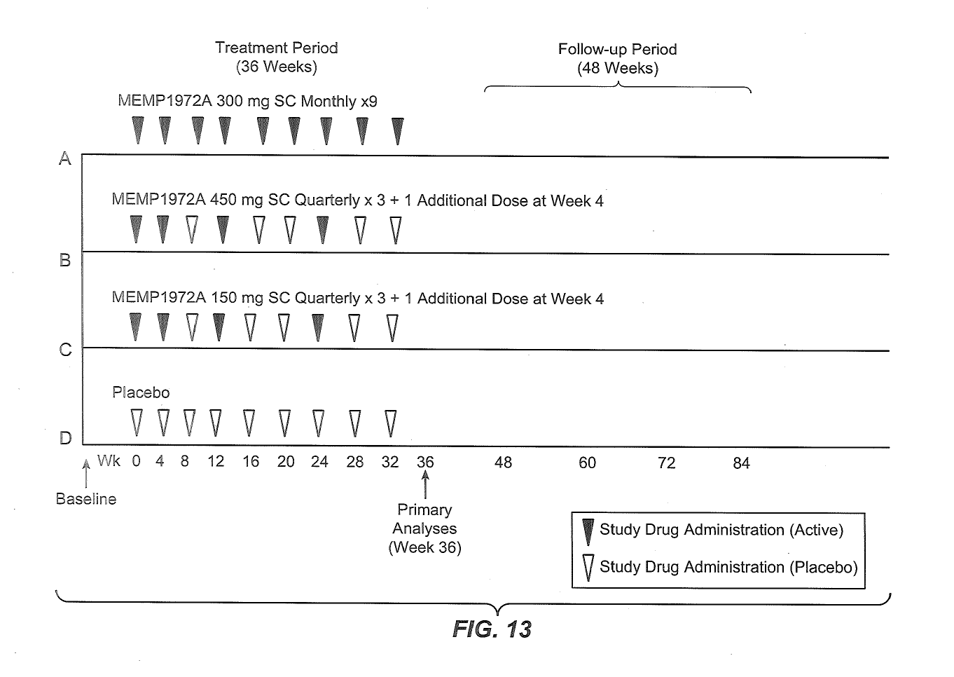

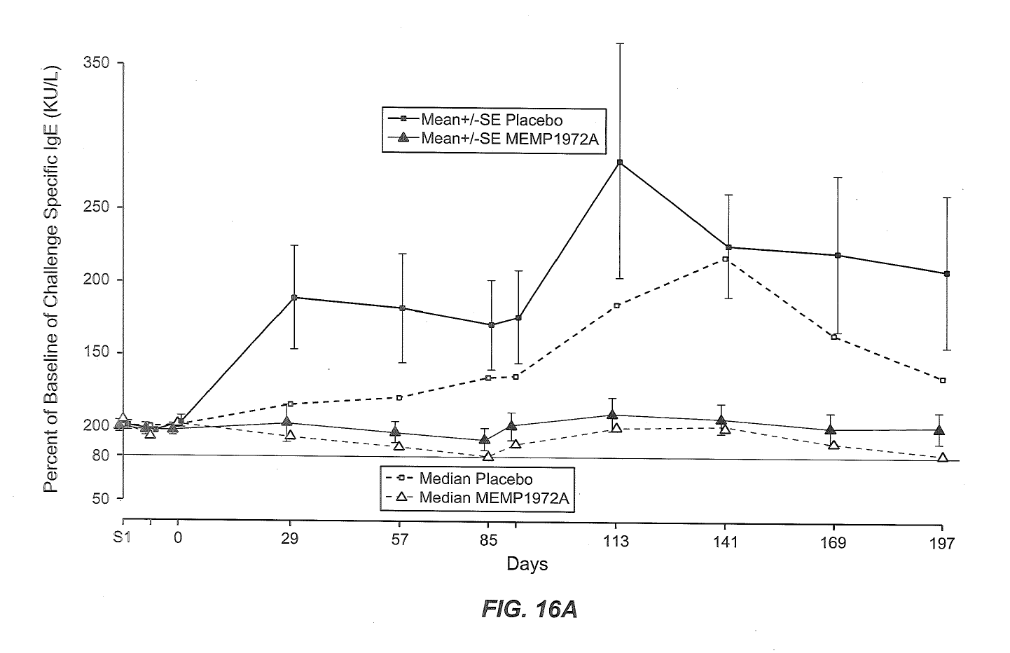

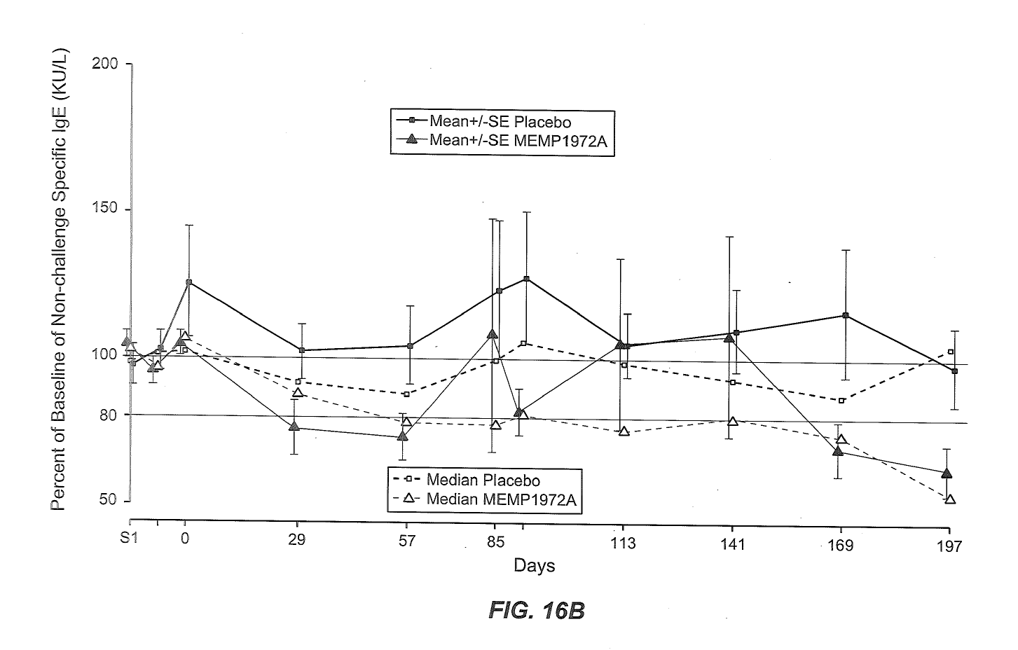

Baseline serum total IgE levels were low (ranging 7-254 IU/ml) but balanced between placebo-(48.5 IU/ml) and MEMP1972A- (57 IU/ml) treated patients. In the placebo group, whole-lung allergen challenge induced an increase in both total and allergen-specific IgE (FIGS. 9A and C). By day 29, allergen challenges administered during screening increased baseline total and allergen-specific IgE by 117% and 188%, respectively. By day 113, the second allergen challenge, administered on day 86, resulted in further increases of baseline total and allergen specific IgE of 141% and 308%, respectively. MEMP1972A treatment completely prevented this allergen-induced increase in serum total and allergen-specific IgE. Allergen inhalation challenge at screening and week 12 in the treatment group induced an approximate 2-fold increase in serum allergen-specific IgE, which was completely blocked by MEMP1972A treatment (p<0.01 vs. placebo) (FIG. 9A). No increase in allergen-specific IgE was observed at any time in MEMP1972A-treated patients. Additionally, irrelevant or non-challenge specific IgE evaluations demonstrated that there was no increase in the irrelevant or non-challenge allergen-specific IgE levels following challenge in placebo and MEMP1972A-treated patients (FIG. 9B). At Day 85 (pre-challenge), the median percent reduction in IgE levels was approximately 20% from baseline for both total IgE and specific (challenge relevant allergen or non-challenge allergens) IgE levels in the MEMP1972A treated subjects (FIGS. 16A and B). At Day 197, the median percent reduction in IgE levels was approximately 20% from baseline for both total IgE and specific challenge relevant IgE levels and more than 20% from baseline for both total IgE and specific non-challenge IgE levels in the MEMP1972A treated subjects (FIGS. 16A and B). Similarly to the increase in serum allergen-specific IgE, AIC at screening and week 12 induced more than an approximate 10-fold increase in sputum eosinophils, which was reduced by MEMP1972A treatment at week 12 (FIG. 10). On day 86, 7 hours after allergen challenge, sputum eosinophils in placebo treated patients were 16%±4.5% (mean±standard error). In MEMP1972A-treated patients, sputum eosinophil levels 7 hours after allergen challenge on day 86 were 8.0%±2.2%. Additionally, MEMP1972A treatment reduced serum total IgE by approximately 20% from baseline at 8 weeks after initiation of treatment (p<0.01 vs. placebo) (FIGS. 9A and C) and reduced blood eosinophils by approximately 45% at week 28 (FIG. 11). Further analysis with data from additional patients showed that MEMP1972A treatment lead up to a 25% from baseline reduction in serum total IgE at day 197 (FIG. 9D). Blood eosinophils decreased in patients treated with MEMP1972A over time, showing mean reductions in absolute counts of 20% and 28% of baseline levels at week 20 and week 38, respectively. In contrast, blood eosinophil levels in placebo-treated patients were increased in relation to baseline and remained above baseline for the duration of the study. Following allergen challenge, serum levels of the Tₕ2 chemoattractant, CCL17, were significantly increased in the placebo-treated, but not the MEMP1972A-treated patients (FIG. 12). In placebo-treated patients, allergen challenge caused a 116 pg/ml increase in serum CCL17 levels from Day 85 (24 hours prior to allergen challenge) to Day 87 (24 hours after allergen challenge). In patients treated with MEMP1972A, serum CCL17 levels increased by 26 pg/ml following allergen challenge from Day 85 to Day 87. Attenuation of allergen-induced EAR, LAR, serum IgE, serum CCL17 as well as sputum and blood eosinophils following AIC was consistent with the mechanism of action of MEMP1972A. Results from this study suggest that depletion of the M1 prime-expressing B-cell lineage is an effective therapeutic strategy for the treatment of allergic asthma. There was no significant increase of adverse events or serious adverse events in the treatment group as compared to the placebo group. MEMP1972A was well tolerated, with no treatment-emergent adverse events (AEs) in the active group, no cluster of AEs indicative of a toxic effect seen for any organ system, and no serious AEs, severe AEs, or AEs leading to discontinuation of study drug. The most frequent AE preferred term was headache, occurring in five patients; three in the placebo arm and two in the MEMP1972A arm. Other AEs reported in at least two patients in either arm were nasopharyngitis (more frequent in the MEMP1972A group), chest discomfort (two patients in the MEMP1972A group), dizziness and oropharyngeal pain, with two placebo patients each, respectively (Table B).

TABLE B
Common adverse events occurring in at ≧2 patients per group

| | Placebo (n = 14) | MEMP1972A (n = 15) |
|---|---|---|
| Adverse Event | n (%) | n (%) |
| Any adverse event | 8 (57) | 10 (67) |
| Headache | 3 (21) | 2* (13) |
| Nasopharyngitis | 1 (7) | 4* (27) |
| Chest discomfort | 0 | 2* (13) |
| Asthma | 2 (14) | 0 |
| Dizziness | 2 (14) | 0 |
| Hypersensitivity | 2 (14) | 0 |
| Oropharyngeal pain | 2 (14) | 0 |

*All were mild, Grade 1 events

TABLE 6
Pre-challenge LAR FEV₁ subgroup analysis

| | | | Mean LAR | Mean LAR | Mean EAR | Mean EAR |
|---|---|---|---|---|---|---|
| | | | AUC | Max | AUC | Max |
| | | | reduction | reduction vs. | reduction | reduction |
| | | | vs. placebo | placebo | vs. placebo | vs. placebo |
| Group | MEMP1972A N | Placebo N | (90% CI) | (90% CI) | (90% CI) | (90% CI) |
| Subgroup A: | 8 | 7 | 43% (−5, 77) | 33% (−5, 63) | 20% (−10, 43) | 2% (−23, 24) |
| (≧2 | | | | | | |
| consecutive | | | | | | |
| ≧15% drops in | | | | | | |
| pre-challenge | | | | | | |
| LAR FEV₁) | | | | | | |
| Subgroup B: | 10 | 6 | 62% (29, 88) | 44% (15, 65) | 35% (5, 55) | 20% (−4, 39) |
| (≧3 | | | | | | |
| consecutive | | | | | | |
| ≧10% drops in | | | | | | |
| pre-challenge | | | | | | |
| LAR FEV₁) | | | | | | |
| Subgroups A | 11 | 8 | 56% (21, 83) | 41% (14, 62) | 31% (6, 49) | 13% (−9, 32) |
| or B | | | | | | |
| Subgroup C: | 11 | 9 | 54% (10, 84) | 35% (−2, 60) | 31% (7, 49) | 17% (−3, 34) |
| LAR AUC | | | | | | |
| screening | | | | | | |
| ≧10%/hr | | | | | | |
| All subjects | 15 | 13 | 36% (−14, 69) | 13% (−33, 44) | 26% (6, 43) | 11% (−7, 26) |

AUC = area under the curve;
CI = confidence interval;
EAR = early asthmatic response;
FEV₁ = forced expiratory volume in 1 second;
LAR = late asthmatic response

Conclusions

After administration of three monthly doses, MEMP1972A treatment reduced the overall level of asthmatic response compared with placebo. No increases in allergen-specific IgE were observed at any time in MEMP1972A-treated subjects demonstrating persistence of effect post-treatment. The observed reduction in total IgE following MEMP1972A treatment is consistent with data from Phase I studies in healthy volunteers and subjects with allergic rhinitis. MEMP197A was well tolerated, with no significant differences in AEs observed between the treatment groups. These data support continued investigation of MEMP1972A as a treatment for asthma and a Phase IIb study of MEMP1972A in subjects with poorly controlled asthma, despite ICS and LABA treatment, was planned.

### Example 3

### Effect of an Anti-M1 Prime Monoclonal Antibody, MEMP1972A, in a Phase 2b Proof-of-Concept Study for a Quarterly Dosage Regimen in Subjects with Asthma

MEMP1972A is evaluated in a Phase 2b proof-of-concept study in patients with allergic asthma inadequately controlled on inhaled steroids and a second controller. This randomized, double-blind, placebo-controlled, 36-week study is performed to evaluate the efficacy, safety and tolerability of a MEMP1972A dosing regimen in subjects with allergic asthma who remain inadequately controlled on chronic therapy with high dose inhaled corticosteroids and a second controller medication. About 560 subjects from an 18 to 75 year old target population are randomized (1:1:1:1) into four cohorts (A-D) to receive placebo or MEMP1972A (FIG. 13). The 140 subjects in Cohort A are given a subcutaneous (SC) dose 300 mg of the study drug every four weeks over the course of 36 weeks (Weeks 0, 4, 8, 12, 16, 20, 24, 28, and 32). The 140 subjects in Cohort B are given a total of four 450 mg SC doses of the study drug with active doses at quarterly intervals as well as an extra dose at Week 4 (Weeks 0, 4, 12, and 24). The 140 subjects in Cohort C are given a total of four 150 mg SC doses of the study drug with active doses at quarterly intervals as well as an extra dose at Week 4 (Weeks 0, 4, 12, and 24). The 140 subjects in Cohort D are given a placebo dose every four weeks over the course of the 36 week study (Weeks 0, 4, 8, 12, 16, 20, 24, 28, and 32). The anticipated time on study treatment is 36 weeks, with a 48-week follow-up.

The primary endpoint for the quarterly dosing regimen is the rate of protocol-defined asthma exacerbations resulting in use of systemic steroids over 36 weeks. The rate is estimated by the total number of protocol-defined exacerbations observed in the group over the treatment period divided by total patient-weeks at risk for the group. For each individual patient, weeks at risk is computed as the number of days between the treatment completion or treatment period discontinuation date and the date of first study drug administration, divided by 7 days. Poisson regression with over dispersion is used in the analysis to assess the treatment effect on the rate of protocol-defined asthma exacerbations. The model adjusts for serum periostin level (<50 ng/mL, ≧50 ng/mL), number of exacerbations requiring use of systemic corticosteroids in the prior 18 months (1, >1), IgE level (≦75 IU/mL, 75-200 IU/mL, >200 IU/mL), and country.

Secondary endpoints are relative change from baseline to Weeks 12 and 36 in pre-bronchodilator FEV₁, change from baseline to Weeks 12 and 36 in the asthma symptoms score and percentage of weeks from Weeks 24 to 36 that asthma is well controlled. “Well-controlled” means no nighttime awakenings due to asthma symptoms and ≦2 days of short acting beta agonist (SABA) use per week, as documented by patient diary.

Exploratory endpoints include the rate of exacerbations beyond Week 36, response at Weeks 36 and 84, FEV₁ and other spirometric measures of lung function change in asthma control, change in asthma symptom scores, and exploratory biomarkers. Exploratory analyses for all outcome measures are performed within the diagnostic subsets (periostin and other pre-specified biomarker candidates).

Safety outcome measure includes incidence of adverse events in 48 weeks and incidence of anti-therapeutic antibodies (ATAs) in 84 weeks. Pharmacokinetics outcome measure includes area under the concentration-time curve (AUC) measured pre- and post-dose of MEMP1972 at weeks 0, 4, 12, 24, and 36.

The primary endpoint for demonstrating the clinical efficacy of the quarterly dosing regimen in the Phase 2b study is ≧55% exacerbation rate reduction as compared to placebo. The secondary endpoint is ≧5% improvement in FEV₁, or reduction in asthma symptom frequency or severity as compared to placebo within 12 weeks of first dose and after 36 weeks of active dosing, or increased percent of well controlled weeks compared to placebo over 24 weeks to 36 weeks of active dosing as demonstrated by a difference of ≧1 well-controlled weeks with control measured by short acting beta agonist (SABA) use and night time awakenings. Clinical efficacy determination of the quarterly dosing regimen is further evaluated in subjects demonstrating primary and secondary endpoints outside of the ranges described above (see Table 7).

TABLE 7
Clinical efficacy criteria for quarterly dosing regimen.

| | Efficacy | | | | |
|---|---|---|---|---|---|
| | Endpoints | Effective | Evaluate | Evaluate | Evaluate |
| Primary | Exacerbation rate | ≧50% | ≧50% | 40-49% | 40-49% |
| Endpoint | reduction | AND | AND | AND | AND |
| | compared to | | | | |
| | placebo | | | | |
| Secondary | Improvement in | ≧5% | <5% | <5% | ≧5% |
| Endpoint | FEV₁ | | | | |
| | OR | OR | AND | AND | OR |
| | Reduction in | Symptom | No symptom | Symptom | Symptom |
| | symptom frequency | Reduction | Reduction | Reduction | Reduction |
| | or severity compared | | | | |
| | to placebo within 12 | | | | |
| | weeks of first dose | | | | |
| | and after 36 weeks | | | | |
| | of active dosing | | | | |
| | OR | OR | AND | OR | OR |
| | Increased percent of | Change in | No change in | Change in | Change in |
| | well controlled | proportion of | proportion of | proportion of | proportion of |
| | weeks compared to | well controlled | well controlled | well controlled | well controlled |
| | placebo over 24 | weeks | weeks | weeks | weeks |
| | weeks to 36 weeks | | | | |
| | of active dosing | | | | |

For this study, the asthma exacerbation event that is used to assess the primary endpoint is defined as new or increased asthma symptoms that lead to hospitalization or to treatment with systemic corticosteroids. The new or increased asthma symptoms include at least one of the following new or increased (if pre-existing) symptoms: wheezing, cough (including changes in sputum production or quality as well as cough frequency), chest tightness, shortness of breath, or nocturnal awakening ascribed to one of the symptoms above. As a result of any of these asthma symptoms, one of the following must be documented: Hospitalization for asthma treatment or treatment with systemic corticosteroids. Treatment with systemic corticosteroids is defined as: treatment with oral, intravenous (IV), or intramuscular (IM) corticosteroids for at least 3 days, or an emergency department visit with at least one dose of IV or IM corticosteroids. The onset or start of an asthma exacerbation is defined by the date of hospitalization or the date treatment with systemic steroids (oral, IM, or IV) began, whichever occurs first. The end of an exacerbation is defined by discontinuation of systemic steroids. Any protocol-defined asthma exacerbation episode that occurs within 7 days of the last dose of systemic corticosteroid (oral, IM, IV) treatment for a prior protocol-defined exacerbation is considered a continuation of the previous exacerbation.

Clinical safety criteria are: 1) acceptable anaphylactic reactions as demonstrated by <or equal to 2 subjects with serious anaphylactic reactions related to Anti-M1 prime; 2) no Anti-M1 prime subjects with Grade 4 injection site reactions; 3) no persistent Grade 4 (<500 cells/mcl) neutropenia or thrombocytopenia (<20,000 cells/mcl) in Anti-M1 prime subjects; and 3) minimal increase in infection rates as demonstrated by less than or equal to 15% Anti-M1 prime subjects over placebo with infections and less than 6% of Anti-M1 prime subjects over placebo with Grade 4 infections. Inclusion criteria for eligible subjects include: age 18 to 75 years; weight ≧40 kg; diagnosis of asthma for at least 12 months; evidence of documented bronchodilator reversibility defined by either 12% or greater β-agonist reversibility using up to 4 puffs albuterol (at screening or with last two years), or PC₂₀ FEV₁ methacholine (provocative concentration of methacholine producing a 20% fall in FEV₁) 8 mg/ml or less (within last 2 years); prebronchodilator FEV₁≧40% and ≦80% predicted at Visit 1; required daily use of ICS (≧400 μg/day total daily dose of fluticasone propionate (FP) or equivalent (Table 8 below) and second controller for a minimum of 3 consecutive months prior to Visit 1; history of at least one asthma exacerbation requiring systemic corticosteroid treatment for at least 3 days (or administered in formulation to provide therapeutic dose ≧72 hours) and no more than 30 days in the 18 months prior to Visit 1; inadequately-controlled asthma at Visits 1 and 2 as documented by asthma control questionnaire score (ACQ) score ≧1.50 at each visit; inadequately controlled asthma despite compliance with asthma controller therapy documented by daily diary during the run-in period as defined by, with complete diary data entry for at least 10 of 14 days prior to randomization, ≧1 nighttime awakening/week and rescue medication use (at least one “puff” of short acting beta agonist [SABA] or nebulized SABA at least 2 days/week); either serum IgE “positive” to at least one “clinically relevant” aeroallergen or a serum total IgE of at least 75 IU/mL; ECG at screening within normal limits.

TABLE 8
Equivalent doses of ICS: relative to
fluticasone propionate 400 μg/day dose

| | 400 μg/d | |
|---|---|---|
| Alternative ICS | FP | |
| FP MDI | 10 | puffs/d |
| 44 μg/puff | | |
| FP MDI | 4 | puffs/d |
| 110 μg/puff | | |
| FP MDI | 2 | puffs/d |
| 220 μg/puff | | |
| FP DPI | 8 | doses/d |
| 50 μg/dose | | |
| FP DPI | 4 | puffs/d |
| 100 μg/dose | | |
| FP DPI | 2 | doses/d |
| 250 μg/dose | | |
| Budesonide DPI | 2 | doses/d |
| 200 μg/dose | | |
| Budesonide CFC | 2 | doses/d |
| 200 μg/dose | | |
| Beclomethasone CFC | 10 | puffs/d |
| 42 μg/puff | | |
| Beclomethasone CFC | 5 | puffs/d |
| 84 μg/puff | | |
| Beclomethasone HFA | 10 | puffs/d |
| 40 μg/puff | | |
| Beclomethasone HFA | 5 | puffs/d |
| 80 μg/puff | | |
| Flunisolide | 2 | puffs/d |
| 250 μg/puff | | |
| Mometasone DPI | 2 | doses/d |
| 220 μg/dose | | |
| Ciclesonide HFA | 5 | puffs/d |
| 80 μg/puff | | |
| Ciclesonide HFA | 2-3 | puffs/d |
| 160 μg/puff | | |
| Triamcinolone MDI | 4 | puffs/d |
| 100 μg/puff | | |

Note:
Dose equivalents derived based on number of micrograms of steroid per actuation without adjustment for potency.
CFC = chlorofluorocarbons;
DPI = dry powder inhaler;
FP = fluticason propionate,
HFA = hydrofluoroalkane;
ICS = inhaled corticosteroid;
MDI = metered dose inhale
d = day.

Subjects with the following criteria are excluded from study entry: Asthma exacerbation requiring systemic steroids in the 30 days prior to Visit 1 or between Visit 1 and Visit 2; >20% relative change in FEV1 between Visit 1 and Visit 2; failed screening for this study more than once (patients may rescreen one time if the reason for screen failure is transient); have pre-existing active lung disease other than asthma; any infection including chronic or latent infections or infections requiring treatment during screening, this includes respiratory tract infections (including, but not limited, to sinus disease and bronchitis); clinically significant medical disease that is uncontrolled despite treatment or is likely to require a change in therapy during the study or is of unknown etiology (e.g., chronic liver disease) including chronic diseases that may exacerbate (e.g., inflammatory bowel disease or rheumatoid arthritis), a known malignancy (newly diagnosed or inadequately treated) or current evaluation for a potential malignancy, known immunodeficiency, including but not limited to HIV infection, regardless of treatment status; elevated IgE levels for reasons other than allergy (including, but not limited to, parasitic infections, hyperimmunoglobulin E syndrome, bronchopulmonary aspergillosis and Wiskott-Aldrich syndrome); any condition that contraindicates the use of an investigational drug or that may affect the interpretation of the results or the patient's ability to participate including past or current substance abuse, lack of compliance, ongoing or recent participation in another investigational trial (within 30 days prior to Visit 1 or 5 half-lives of the investigational drug, whichever is longer); history of significant exposure to water-borne parasites (within 6 weeks prior to Visit 1) and/or have recent diarrheal illness of indeterminate etiology (within 3 months prior to Visit 1), an exception is made if a stool test is obtained and the results are negative and documented; former smoker with >10 pack year history or current smoker (former smoker must have stopped smoking more than 12 months prior to Visit 1); history of anaphylaxis (from any trigger) or allergic reaction during the use (or possible use if blinded study) of a monoclonal antibody; use of any excluded concomitant therapies (see Table 9); men and women who are not willing to use a highly effective method of contraception through at least Week 60 of the study; women who are not pregnant or lactating at the time of Visit 1; and absolute neutrophil count <1.5×10⁹/L during screening.

TABLE 9
Concomitant Medications and Windows

| Medication | Prohibited Period (1 month = 30 days) |
|---|---|
| ICS | No change to ICS dose within 30 days prior to |
| | Visit 1. No anticipated changes in ICS |
| | throughout the study |
| Extended release (depot injection) steroids | Within 6 months prior to Visit 1 and throughout |
| | the study |
| Maintenance oral steroids (daily or QOD)^{a} | Within 6 months prior to Visit 1 and continuing |
| | throughout the study |
| Any oral, IV or IM steroids^{a} | Within 30 days prior to Visit 1 and continuing |
| | throughout the study |
| Allergen immunotherapy | No change to immunotherapy or new |
| | immunotherapy within 3 months prior to Visit 1. |
| | No anticipated changes in immunotherapy |
| | throughout the study. |
| Leukotriene modifiers | No change in dose, including initiation of |
| | therapy, 30 days prior to Visit 1. |
| | No anticipated changes in leukotriene dose |
| | throughout the study. |
| Any immunomodulatory agents (including but | Within 3 months or 5 drug half-lives |
| not limited to methotrexate, troleandomycin, | (whichever is longer) prior to Visit 1 and |
| oral gold, cyclosporin, anti-TNF therapy, and | continuing throughout the study |
| mycophenolate) with the exception of | |
| corticosteroids.^{c} | |
| Terbutaline or ipratropium | Between Visits 1 and 20 |
| Monoclonal antibody therapy | Within 5 months prior to Visit 1^{b} and continuing |
| | throughout the study |
| Omalizumab | Within 5 months prior to Visit 1 and continuing |
| | throughout the study |
| Investigational drug (including investigational | Within 30 days or 5 half-lives (whichever is |
| use of a formulation of an approved drug) | longer) prior to Visit 1 and continuing |
| | throughout the study |

ICS = inhaled corticosteroids;
IM = intramuscular;
IV = intravenous;
QOD = every other day
Note:
Patients who take any extended release (depot injection) steroids, immunomodulatory agents, or investigational drugs must be discontinued from study drug, but should remain in the study for continued observation and assessments. Oral, IV or IM (nondepot preparation) steroids, terbutali or ipratropium may be used to treat an asthma exacerbation while the patient continues to receive study drug, however the duration of treatment must be less than 30 days or study drug must be discontinued. Steroids used to treat indications other than asthma are prohibited. If necessary for treatment of another indication, the duration of treatment must remain less than 30 days or study drug must be discontinued. The doses of ICS, allergen immunotherapy, and leukotriene modifiers should remain stable throughout the study. However, if the doses of these medications are changed for a brief period (less than 30 days) during the treatment period, the patient may be allowed to continue study drug. If the dose is changed for a period lasting 30 days or more, study drug must be discontinued. The patient should remain in the study for continued observation and assessments. The Medical Monitor should be consulted to ensure there are no safety risks associated with continuing study drug in addition to concomitant medications.
^{a}A standard short course(from 3 to 30 days) of oral or intravenous steroid agents for the treatment of an asthma exacerbation is appropriate and permissible anytime ≧30 days prior to Visit 1 and after Visit 2. Between Visits 1 and 2 oral or IV or IM steroid use or change in ICS dose renders the patient ineligible.
^{b}Patients participating in a trial of an investigational monoclonal antibody which has not been unblinded, should be assumed to have received the active agent.
^{c}Vaccinations are permitted before or during the study. To more clearly interpret any reaction that occurs subsequent to an injection (of study drug or vaccine), it is recommended that the vaccination not occur on the same day as study drug administration. If both must be administered on the same day, the injection site for the vaccination should be remote (different limb) from the study drug administration site(s).

What is claimed is:

1. A method of treating or preventing an IgE-mediated disorder comprising administering to a human patient a therapeutically effective amount of an anti-IgE antibody that binds the M1′ segment of a human IgE, wherein an interval between administrations of the antibody is about one month or longer.

2. (canceled)

3. The method of claim 1, wherein the interval between administrations is about three months.

4-6. (canceled)

7. The method of claim 1, wherein the antibody is administered at a dosage from about 150 mg to about 450 mg per dose.

8. The method of claim 1, wherein the antibody is administered at a dosage about 150 mg, about 300 mg, or about 450 mg per dose.

9. The method of claim 1, wherein the antibody is administered subcutaneously or intravenously.

10. The method of claim 1, wherein the interval between administrations is about three months with an additional administration at week 4 after the first administration.

11. The method of claim 1, wherein the serum total IgE in the human patient is reduced relative to baseline after the antibody treatment.

12. The method of claim 1, wherein the allergen-specific IgE in the human patient is reduced relative to baseline after the antibody treatment.

13. The method of claim 1, wherein an allergen-induced increase in serum total IgE in the human patient is prevented or reduced after the antibody treatment.

14. The method of claim 1, wherein an allergen-induced increase in allergen-specific IgE in the human patient is prevented or reduced after the antibody treatment.

15. A method of treating or preventing an IgE-mediated disorder comprising administering to a human patient an effective amount an anti-IgE antibody that binds the M1′ segment of a human IgE, wherein the antibody is administered at a dose of about 150 to about 450 mg per dose.

16-17. (canceled)

18. A method of reducing serum total IgE in a human relative to baseline comprising administering to a human patient an effective amount of an anti-IgE antibody that binds the M1′ segment of a human IgE, wherein an interval between administrations of the antibody is about one month or longer.

19. The method of claim 18, wherein the serum total IgE is reduced by at least about 20% from the baseline level.

20-22. (canceled)

23. The method of claim 18, wherein the reduction of the serum total IgE is sustained for at least three months after the last administration of the antibody.

24. (canceled)

25. A method of preventing or reducing an allergen-induced increase in serum total IgE in a human patient comprising administering to a human patient an effective amount of an anti-IgE antibody that binds to the M1′ segment of a human IgE.

26. (canceled)

27. The method of claim 25, wherein an interval between administrations of the antibody is about three months.

28. The method of claim 25, wherein the antibody is administered at a dose of about 150 to about 450 mg per dose.

29. The method of claim 25, wherein an allergen-induced increase in allergen-specific IgE is prevented or reduced.

30-33. (canceled)

34. The method of claim 1, wherein the IgE-mediated disorder is allergic rhinitis, allergic asthma, or non-allergic asthma.

35. The method of claim 1, wherein the human patient has allergic asthma that is inadequately controlled by a high-dose inhaled or oral corticosteroids in combination with a second controller.

36. The method of claim 35, wherein the second controller is a bronchodilator or an anti-leukotriene agent.

37. The method of claim 1, further comprising administering to the human patient a second drug in conjunction with the antibody for treating or preventing an IgE-mediated disorder, wherein the second drug is selected from the group consisting of: an anti-IgE antibody, an antihistamine, a bronchodilator, a glucocorticoid, an NSAID, a decongestant, a cough suppressant, an analgesic, a TNF-antagonist, an integrin antagonist, an immunosuppressive agent, an IL-4 antagonist, an IL-13 antagonist, a dual IL-4/IL-13 antagonist, a DMARD, an antibody that binds to a B-cell surface marker, and a BAFF antagonist.

38. The method of claim 1, wherein the antibody is administered to the human patient in conjunction with a second method treatment for an IgE-mediated disorder

39. (canceled)

40. The method of claim 1, wherein the antibody is a chimeric, a humanized, or a human antibody.

41-50. (canceled)

51. The method of claim 40, wherein the antibody comprises a heavy chain and a light chain variable region, wherein the heavy chain variable region comprises an HVR-H1, HVR-H2 and HVR-H3, and the light chain variable region comprises HVR-L1, HVR-L2 and HVR-L3, and wherein (a) the HVR-H1 comprises residues 26-35 of SEQ ID NO:29, (b) the HVR-H2 comprises residues 49-66 of SEQ ID NO:29, (c) the HVR-H3 comprises residues 97-106 of SEQ ID NO:29, (d) the HVR-L1 comprises residues 24-39 of SEQ ID NO:19, (e) the HVR-L2 comprises residues 55-61 of SEQ ID NO:19, and (f) the HVR-L3 comprises residues 94-102 of SEQ ID NO:19.

52-57. (canceled)

58. The method of claim 40, wherein the antibody is in a pharmaceutical composition comprising the antibody and a pharmaceutically acceptable carrier.

59. A kit comprising an anti-IgE antibody that binds the M1′ segment of a human IgE and a package insert indicating that the antibody is administered to a human patient for treating or preventing an IgE-mediated disorder, wherein an interval between administrations of the antibody to the human patient is about one month or longer.

60. A kit comprising an anti-IgE antibody that binds the M1′ segment of a human IgE and a package insert indicating that the antibody is administered to a human patient for treating or preventing an IgE-mediated disorder, wherein the antibody is administered at a dosage from about 150 mg to about 450 mg per dose.

61-70. (canceled)


SEQUENCE LISTING (43 sequences)

SEQ ID NO: 1 - protein, 431 aa, Homo sapiens
ILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGLAGGSAQSQRAPDRVLCHSGQQQGLPRAAGGSVPHPRCHCGAGRADWPGPPELDVCVEEAEGEAPWTWTGLCIFAALFLLSVSYSAALTLLMVQRFLSATRQGRPQTSLDYTNVLQPHA

SEQ ID NO: 2 - protein, 432 aa, Macaca mulatta
ILQSSCDDDGHFPPTIQLLCLISGYTPGAINVTWLENGQVMKVNSPTPPATQEGELASTQSEFTLAQKHWLSDRTYTCQVTYQGTTYNDSTKKCADSNPRGVSAYLSRPSPFDLFISKSPTITCLVVDLAPSKETVNLTWSRASGKPVPHIPATEKKQQRNGTLTVTSILPVVTQDWIEGETYQCRVTHPHLPRALVRSMTKTSGPRAAPEVYVFATPEKLESRDKRTLACLIQNFMPEDISVQWLHSDVQLPDARHSVTQPRKTKGSGFFVFSRLEVTKAEWEQKDEFICRAVHEAASPSWIVQQAVSVNPGLAGGSAQSQRAPDRVLCHSEQQQGLPRAARGSVPDHHCHCGAGRADWPGLPELDLCVEEAESEVLWTWTGLCIFATLFLLSVSYSAAITLLMVQRFLSATRQGRPQTSLDYTNVLQPHA

SEQ ID NO: 3 - protein, 431 aa, Macaca fascicularis
ILQSSCDDDGHFPPTIQLLCLISGYTPGAINVTWLENGQVMKVNSPTPPATQEGELASTQSEFTLAQKHWLSDRTYTCQVTYQGTTYNDSTKKCANSNPRGVSAYLSRPSPFDLFISKSPTITCLVVDLAPSKETVNLTWSRASGKPVPHTPATEKKQRNGTLTVTSILPVVTQDWIEGETYQCRVTHPHLPRALVRSMTKTSGPRAAPEVYVFATPEKLESRDKRTLACLIQNFMPEDISVQWLHSDVQLPDARHSVTQPRKTKGSGFFVTSRLEVTKAEWEQKDEFICRAVHEAASPSWIVQQAVSVNPGLAGGSAQSQRAPDRVLCHSEQQQGLPRAARGSVPDHRCHCGAGRADWPGLPELDLCVEEAESEVLWTWTGLCIFATLFLLSVSYSAAITLLMVQRFLSVTRQGRPQTSLDYTNILQPHA

SEQ ID NO: 4 - protein, 15 aa, Homo sapiens
SAQSQRAPDRVLCHS

SEQ ID NO: 5 - protein, 15 aa, Homo sapiens
RAPDRVLCHSGQQQG

SEQ ID NO: 6 - protein, 15 aa, Homo sapiens
GQQQGLPRAAGGSVP

SEQ ID NO: 7 - protein, 14 aa, Homo sapiens
PRAAGGSVPHPRCH

SEQ ID NO: 8 - protein, 108 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 9 - protein, 108 aa, Mus musculus
DIQMTQTTSSLSASLGDRVTITCRSSQDISNSLNWYQQKPDGPVKLLIYSTSRLHSGVPSRFSGSGSGTDYSLTISNLEQEDLATYFCQQGHTLPWTFGGGTKVEIKR

SEQ ID NO: 10 - protein, 108 aa, Artificial sequence
  sequence is synthesized
DIQMTQSPSSLSASVGDRVTITCRSSQDISNSLNWYQQKPGKAPKLLIYSTSRLHSGVPSRFSGSGSGTDFTLTISSLQPEDFATYYCQQGHTLPWTFGQGTKVEIKR

SEQ ID NO: 11 - protein, 108 aa, Artificial sequence
  sequence is synthesized
DIQMTQSPSSLSASVGDRVTITCRSSQDISNALNWYQQKPGKAPKLLIYSTSRLHSGVPSRFSGSGSGTDFTLTISSLQPEDFATYYCQQGHTLPWTFGQGTKVEIKR

SEQ ID NO: 12 - protein, 108 aa, Artificial sequence
  sequence is synthesized
DIQMTQSPSSLSASVGDRVTITCRSSQDISNYLNWYQQKPGKAPKLLIYSTSRLHSGVPSRFSGSGSGTDFTLTISSLQPEDFATYYCQQGHTLPWTFGQGTKVEIKR

SEQ ID NO: 13 - protein, 108 aa, Artificial sequence
  sequence is synthesized
DIQMTQSPSSLSASVGDRVTITCRSSQDISSSLNWYQQKPGKAPKLLIYSTSRLHSGVPSRFSGSGSGTDFTLTISSLQPEDFATYYCQQGHTLPWTFGQGTKVEIKR

SEQ ID NO: 14 - protein, 108 aa, Artificial sequence
  sequence is synthesized
DIQMTQSPSSLSASVGDRVTITCRSSQDISQSLNWYQQKPGKAPKLLIYSTSRLHSGVPSRFSGSGSGTDFTLTISSLQPEDFATYYCQQGHTLPWTFGQGTKVEIKR

SEQ ID NO: 15 - protein, 114 aa, Mus musculus
DIVMSQSPSSLTVSVGEKVTLSCKSSQTLLYSTNQKNYLAWYQQKPGQSPKLLIYWASTRESGVPDRFTGSGSGTDFTLTISSVKAEDLAVYYCQQYYSYPYTFGGGTKVEIKR

SEQ ID NO: 16 - protein, 114 aa, Artificial sequence
  sequence is synthesized
DIQMTQSPSSLSASVGDRVTITCKSSQTLLYSTNQKNYLAWYQQKPGKAPKLLIYWASTRESGVPSRFSGSGSGTDFTLTISSLQPEDFATYYCQQYYSYPYTFGQGTKVEIKR

SEQ ID NO: 17 - protein, 113 aa, Mus musculus
DIVLTQTPLSLPVSLGDQASISCRSSQSLVHNNGNTYLHWYLQKPGQSPKLLIYKVSNRFSGVPDRFSGSGSGTDFTLKISRLEAEDLGVYFCSQNTLVPWTFGGGTKVEIKR

SEQ ID NO: 18 - protein, 113 aa, Artificial sequence
  sequence is synthesized
DIQMTQSPSSLSASVGDRVTITCRSSQSLVHNNGNTYLHWYQQKPGKAPKLLIYKVSNRFSGVPSRFSGSGSGTDFTLTISSLQPEDFATYYCSQNTLVPWTFGQGTKVEIKR

SEQ ID NO: 19 - protein, 113 aa, Artificial sequence
  sequence is synthesized
DIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKR

SEQ ID NO: 20 - protein, 113 aa, Homo sapiens
EVQLVESGGGLVQPGGSLRLSCAASGFTFSSYAMSWVRQAPGKGLEWVSVISGDGGSTYYADSVKGRFTISRDNSKNTLYLQMNSLRAEDTAVYYCARGFDYWGQGTLVTVSS

SEQ ID NO: 21 - protein, 112 aa, Mus musculus
EVQLQQSGPELVKPGASVKMSCKASGYTFTDYYMMWVKQSHGKSLEWIGDINPNNYDTSYNQKFKGKATLTVDKSSSTAYIQLNSLTSEDSAVYYCASKAYWGQGTLVTVSS

SEQ ID NO: 22 - protein, 112 aa, Artificial sequence
  sequence is synthesized
EVQLVESGGGLVQPGGSLRLSCAASGYTFTDYYMMWVRQAPGKGLEWVGDINPNNYDTSYNQKFKGRFTISADTSKNTAYLQMNSLRAEDTAVYYCASKAYWGQGTLVTVSS

SEQ ID NO: 23 - protein, 112 aa, Artificial sequence
  sequence is synthesized
EVQLVESGGGLVQPGGSLRLSCAASGYTFTDYYIMWVRQAPGKGLEWVGDINPNNYDTSYNQKFKGRFTISADTSKNTAYLQMNSLRAEDTAVYYCASKAYWGQGTLVTVSS

SEQ ID NO: 24 - protein, 122 aa, Mus musculus
QVQLQQSGAELVRPGASVTLSCKASGYTFIDYEMHWVKQTPVHGLEWIGAIDPETGTTAYNQKFKGKATLTAAKSSSTAYMELRSLTSEDSAVYYCTRLRPHYDYDNAMDSWGQGTTLTVSS

SEQ ID NO: 25 - protein, 122 aa, Artificial sequence
  sequence is synthesized
EVQLVESGGGLVQPGGSLRLSCAASGYTFIDYEMHWVRQAPGKGLEWVGAIDPETGTTAYNQKFKGRFTISADTSKNTAYLQMNSLRAEDTAVYYCTRLRPHYDYDNAMDSWGQGTLVTVSS

SEQ ID NO: 26 - protein, 117 aa, Mus musculus
EVKLVESGGGLVQPGGSRKLSCAASGFTFSDYGMAWVRQAPGKGPEWVAFISDLAYTIYYADTVTGRFTISRENAKNTLYLEMSSLRSEDTALYYCARDNWDAMDYWGQGTSVTVSS

SEQ ID NO: 27 - protein, 117 aa, Artificial sequence
  sequence is synthesized
EVQLVESGGGLVQPGGSLRLSCAASGFTFSDYGMAWVRQAPGKGLEWVAFISDLAYTIYYADTVTGRFTISRDNSKNTLYLQMNSLRAEDTAVYYCARDNWDAMDYWGQGTLVTVSS

SEQ ID NO: 28 - protein, 117 aa, Artificial sequence
  sequence is synthesized
EVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDALDYWGQGTLVTVSS

SEQ ID NO: 29 - protein, 117 aa, Artificial sequence
  sequence is synthesized
EVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVSS

SEQ ID NO: 30 - protein, 117 aa, Artificial sequence
  sequence is synthesized
EVQLVESGGGLVQPGGSLRLSCAASGFTFSDYGMAWVRQAPGKGLEWVAFISDLAYTIYYADTVTGRFTISRDNSKNTLYLQMNSLRAEDTAVYYCARDNWDALDYWGQGTLVTVSS

SEQ ID NO: 31 - protein, 25 aa, Homo sapiens [sequence omitted]

SEQ ID NO: 32 - protein, 14 aa, Homo sapiens
WVRQAPGKGLEWVA

SEQ ID NO: 33 - protein, 32 aa, Homo sapiens
RFTISRDDSKNTLYLQMNSLRAEDTAVYYCAR

SEQ ID NO: 34 - protein, 11 aa, Homo sapiens
WGQGTLVTVSS

SEQ ID NO: 35 - protein, 23 aa, Homo sapiens
DIQMTQSPSSLSASVGDRVTITC

SEQ ID NO: 36 - protein, 15 aa, Homo sapiens
WYQQKPGKAPKLLIY

SEQ ID NO: 37 - protein, 32 aa, Homo sapiens
GVPSRFSGSGSGTDFTLTISSLQPEDFATYYC

SEQ ID NO: 38 - protein, 11 aa, Homo sapiens
FGQGTKVEIKR

SEQ ID NO: 39 - protein, 447 aa, Artificial sequence
  sequence is synthesized
EVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 219 aa, Artificial sequence
  sequence is synthesized
DIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 19 bp, Artificial sequence
  sequence is synthesized
gtggcagagcaccctatcc

SEQ ID NO: 42 - DNA, 17 bp, Artificial sequence
  sequence is synthesized
cagcgagcggtgtctgt

SEQ ID NO: 43 - DNA, 14 bp, Artificial sequence
  sequence is synthesized
ccagcccgggattt